(19) United States
(12) Patent Application Publication
(10) Pub. No.: US 2008/0176744 A1
(43) Pub. Date: Jul. 24, 2008

(54) Use of 4-Aminopyrimidines for Controlling Harmful Fungi, Novel 4-Aminopyrimidines, Processes for Their Preparation and Compositions Comprising Them

(75) Inventors: Anja Schwogler, Mannheim (DE); Joachim Rheinheimer, Ludwigshafen (DE); Wassilios Grammenos, Ludwigshafen (DE); Thomas Grote, Wachenheim (DE); Udo Hunger, Mannheim (DE); Bernd Muller, Frankenthal (DE); Peter Schafer, Ottersheim (DE); Frank Schieweck, Hessheim (DE)

Correspondence Address: HUTCHISON LAW GROUP PLLC, PO BOX 31686, RALEIGH, NC, 27612, US

(73) Assignee: BASF Aktiengesellschaft, Ludwigshafen (DE)

(21) Appl. No.: 11/885,822

(22) PCT Filed: Mar. 8, 2006
(86) PCT No.: PCT/EP06/60557

§ 371 Date: Sep. 7, 2007

(30) Foreign Application Priority Data
Mar. 10, 2005 (DE) 102005011583.7

(51) Int. Cl. A01N 43/54; C07D 239/42; A01C 1/06

(52) U.S. Cl. 504/100; 544/326; 514/256

(57) ABSTRACT

The invention relates to the use of 4-aminopyrimidines of formula (I), in which R¹═H, halogen, cyano, alkyl, alkylhalide, alkenyl, alkinyl, cycloalkyl, alkoxy, alkoxyalkyl, benzyloxyalkyl, alkoxyalkenyl or alkoxyalkinyl, R²═H, halogen, cyano, alkyl, alkylhalide, alkenyl, alkinyl, cycloalkyl, alkoxy, alkoxyalkyl and alkylthioalkyl, whereby the hydrocarbon chain in R¹ and/or R² can be substituted as given in the description, R¹ and R² can form, together with the carbon atom to which they are bonded, a 5-7-membered ring which can contain one to three same or different heteroatoms selected from the group O, N or S, R³═H, halogen, cyano, hydroxy, mercapto, azido, alkyl, alkenyl, alkinyl, alkylhalide, —O-D, —S(O)ₘ-D, ON═CR^{a}R^{b}, CR^{c}═NOR^{a}, NR^{c}N═CR^{a}R^{b}, NR^{a}R^{b}, NR^{c}NR^{a}R^{b}, NOR^{a}, NR^{c}C(═NR^{c})NR^{a}R^{b}, NR^{c}C(═O)NR^{a}R^{b}, NR^{a}CN, —NR^{a}C(═O)R^{c}, NR^{a}C(═NOR^{c})R^{c′}, OC(═O)R^{a}, C(═NOR^{c})NR^{a}R^{b}, CR^{c}(═NNR^{a}R^{b}), C(═O)NR^{a}R^{b}, C(═O)R^{a}, CO₂R^{a}, C(═O)NR^{z}R^{b}, C(═O)—N—OR^{b}, C(═S)—NR^{z}R^{b}, C(═O)NR^{a}—NR^{z}R^{b}, C(═N—NR^{z}R^{c})NR^{a}R^{b}, C(═NOR^{b})R^{a}, C(═N—NR^{z}R^{b})R^{a}, CR^{a}R^{b}—OR^{z}, CR^{a}R^{b}—NR^{z}R^{c}, ON(═CR^{a}R^{b}), NR^{a}(C(═O)R^{b}), NR^{a}(C(═O)OR^{b}), C(═NR^{a})NR^{z}R^{b}, C(═0)—NR^{z}R^{b}) NR^{a}(C(═O)—NR^{z}R^{b}), NR^{a}(C(═NR^{c})R^{b}), NR^{a}(N═CR^{c}R^{b}), NR^{a}—NR^{z}R^{b}, —NR^{z}—OR^{a}, NR^{a}(C(═NR^{c})—NR^{z}R^{b}), NR^{a}(C(═NOR^{c})R^{b}) D=alkyl, alkenyl, alkinyl, alkylhalide, cycloalkyl, five- or six-membered saturated, partly-unsaturated or aromatic mono- or bicyclic heterocycles, containing one to four heteroatoms from the group O, N or S, one of the groups G1 or G2, whereby m, x, R^{a}, R^{b}, R^{c}, R^{d}, R^{e}, R^{z}, Y, Z are as defined in the description and the aliphatic, alicyclic or aromatic groups R³, R^{a}, R^{b}, R^{c}, R^{d} or R^{e} can be substituted as given in the description for the prevention of fungal pests, novel 4-aminopyridines, method for production of said compounds and agents comprising the same.

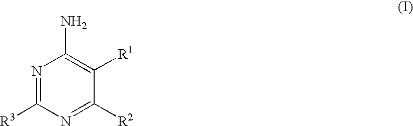

The present invention relates to the use of 4-aminopyrimidines of the formula I

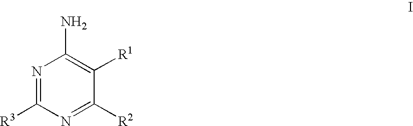

in which the substituents are as defined below:
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl and C₁-C₁₂-alkylthio-C₁-C₁₂-alkyl,
   where the carbon chains in R¹ and/or R² may be substituted by one to four identical or different groups R^{α}:
   R^{α} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, NR^{a}R^{b}, phenyl, C₁-C₆-alkylphenyl;
      R^{a}, R^{b} independently of one another are hydrogen, C₁-C₆-alkyl, C₂-C₆-alkenyl, C₂-C₆-alkynyl, C₃-C₆-cycloalkyl or C₄-C₆-cycloalkenyl;
      where the groups R^{α} may be substituted by one to four groups R^{β}:
      R^{β} is halogen, cyano, hydroxyl, mercapto, C₁-C₁₀-alkyl, C₁-C₁₀-haloalkyl, C₂-C₁₀-alkenyl, C₂-C₁₀-alkynyl and C₁-C₆-alkoxy;
         R¹ and R² together with the carbon atoms to which they are attached may form a five- to seven-membered ring which may contain one to three identical or different heteroatoms from the group consisting of O, N and S;
R³ is hydrogen, halogen, cyano, hydroxyl, mercapto, azido, C₁-C₈-alkyl, C₂-C₈-alkenyl, C₂-C₈-alkynyl, C₁-C₆-haloalkyl, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, —NR^{c}C(═NR^{c′})NR^{a}R^{b}, —NR^{c}C(═O)NR^{a}R^{b}, —NR^{a}CN, NR^{a}C(═O)R^{c}, —NR^{a}C(═NOR^{c})R^{c′}, —OC(═O)R^{a}, —C(═NOR^{c})NR^{a}R^{b}, —CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, —C(═O)NR^{a}—NR^{z}R^{b}, —C(═N—NR^{z}R^{c})NR^{a}R^{b}, —C(═NOR^{b})R^{a}, C(═N—NR^{z}R^{b})R^{a}CR^{a}R^{b}—OR^{z}, —CR^{a}R^{b}—NR^{z}R^{c}, —ON(═CR^{a}R^{b}), —NR^{a}(C(═)R^{b}), —NR^{a}(C(═O)OR^{b}), —NR^{a}(C(═O)—NR^{z}R^{b}), —NR^{a}(C(═NR^{c})R^{b}), —NR^{a}(N═CR^{c}R^{b}), NR^{a}—NR^{z}R^{b}, —NR^{z}—OR^{a}, —NR^{a}(C(═NR^{c})—NR^{z}R^{b}), —NR^{a}(C(═NOR^{c})R^{b}),
   D is C₁-C₈-alkyl, C₃-C₈-alkenyl, C₃-C₈-alkynyl, C₁-C₆-haloalkyl, C₃-C₈-cycloalkyl;
   m is 0, 1 or 2;
   R^{z} is a group R^{a} which may be attached directly or via a carbonyl group;
   R^{c} is one of the groups mentioned under R^{a}, R^{b};
   a five- or six-membered saturated, partially unsaturated or aromatic mono- or bi-cyclic heterocycle which contains one to four heteroatoms from the group consisting of O, N and S,
   one of the groups G1 or G2

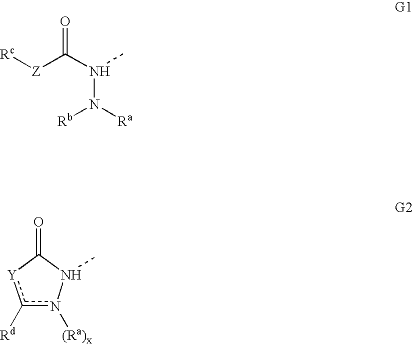

where
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups mentioned under R^{a}, R^{b} or, together with the carbon to which it is attached, may be a carbonyl group;
   Z is oxygen or N—R^{c};
   Y is C(H)—R^{e}, C—R^{e}, N—N(H)—R^{c} or N—R^{c};
   R^{e} is halogen, cyano or one of the groups mentioned under R^{a}, R^{b};
[symbol] is a double or a single bond;
where the aliphatic, alicyclic or aromatic groups R³, R^{a}, R^{b}, R^{c}, R^{d} or R^{e} may be partially or fully halogenated or may carry one to four groups R^{A}:
R^{A} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, OH, SH, two vicinal groups R^{A} may be (═O) or (═S), C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N—OA), N(A′)A, N(A′)—C(═O)-A, N(A″)—C(═O)—N(A′)A, S(═O)ₘ-A, S(═O)ₘ—O-A or S(═O)ₘ—N(A′)A,
   A, A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the groups may be partially or fully halogenated or may be substituted by cyano or C₁-C₄-alkoxy, or A and A′ together with the atoms to which they are attached are a five- or six-membered saturated, partially unsaturated or aromatic heterocycle which contains one to four heteroatoms from the group consisting of O, N and S;
   where the aliphatic, alicyclic or aromatic groups R^{A}, A, A′ and A″ for their part may be partially or fully halogenated or may carry one to three groups R^{b},
      for controlling harmful fungi.

Moreover, the invention relates to novel 4-aminopyrimidines, to processes for preparing these compounds and to compositions comprising them.

EP-A 407 899 and EP-A 12 54 903 propose fungicidally and/or microbicidally active aminopyrimidines in a general manner. However, in many cases their activity against phytopathogenic harmful fungi is unsatisfactory. Based on this, it is an object of the present invention to provide compounds having improved activity and/or a wider activity spectrum.

We have found that this object is achieved by the compounds defined at the outset. Furthermore, we have found processes and intermediates for their preparation, compositions comprising them and methods for controlling harmful fungi using the compounds I.

The compounds of the formula I differ from those in the abovementioned publications by the specific embodiment of the substituents in positions 4, 5 and 6 of the pyrimidine ring.

Compared to the known fungicidal compounds, the compounds of the formula I are more effective against harmful fungi.

The novel compounds of the formula I can be obtained by different routes.

Advantageously, the compounds of the formula I are obtained by converting substituted β-ketoesters of the formula II with thiourea of the formula III to give 2-thio-4-hydroxy-pyrimidines of the formula IV. The variables in formulae II and IV are as defined formula I and the group R in formula II is C₁-C₄-alkyl; for practical reasons, preference is given here to methyl, ethyl or propyl.

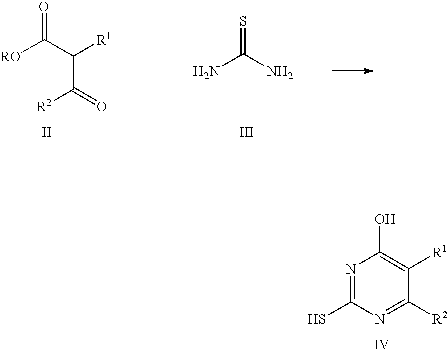

The reaction of the substituted β-ketoesters of the formula II with thiourea of the formula III can be carried out in the presence or absence of solvents. It is advantageous to use solvents to which the starting materials are substantially inert and in which they are completely or partially soluble.

Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines and mixtures of these solvents with water.

Suitable catalysts are bases as mentioned above or acids such as sulfonic acids or mineral acids. With particular preference, the reaction is carried out in the absence of a solvent or in chlorobenzene, xylene, dimethyl sulfoxide or N-methylpyrrolidone.

Particularly preferred bases are tertiary amines, such as triisopropylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), 81ff].

The bases are generally employed in catalytic amounts; however, they can also be employed in equimolar amounts, in excess or, if appropriate, as solvent.

The β-ketoesters of the formula II can be prepared as described in Organic Synthesis Coll. Vol. 1, p. 248, and/or they are commercially available.

Using alkylating agents D-X, such as alkyl halides, preferably methyl chloride or methyl bromide, or dimethyl sulfate or methyl methanesulfonate, the 2-thio-4-hydroxypyrimidines of the formula IV are converted into the thioethers V. The reaction can be carried out in water or else in a dipolar aprotic solvent, such as, for example, N,N-dimethylformamide [cf. U.S. Pat. No. 5,250,689], it is advantageously carried out in the presence of a base, such as, for example, KOH, NaOH, NaHCO₃ and Na₂CO₃ or pyridine. The reaction temperature is preferably 10-60° C.

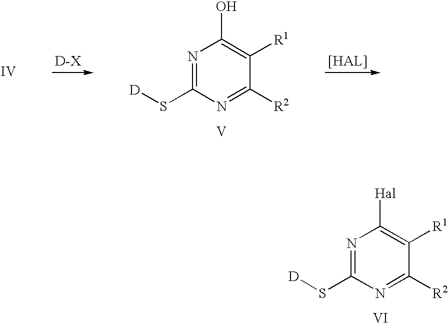

In the formulae V and VI, D is as defined in formula I.

Using halogenating agents, in particular chlorinating agents or brominating agents, the compounds of the formula V are converted into compounds of the formula VI in which Hal is chlorine or bromine, in particular chlorine. Suitable chlorinating agents for the conversion of the hydroxyl compounds V into the compounds VI are, for example, POCl₃, PCl₃/Cl₂ or PCl₅, or mixtures of these reagents. The reaction can be carried out in excess chlorinating agent (POCl₃) or in an inert solvent, such as, for example, acetonitrile, toluene, chlorobenzene or 1,2-dichloroethane. The reaction is preferably carried out in POCl₃.

This reaction is usually carried out between 10 and 180° C. For practical reasons, the reaction temperature usually corresponds to the boiling point of the chlorinating agent (POCl₃) or solvent employed. The process is advantageously carried out with addition of catalytic or substoichiometric amounts of N,N-dimethylformamide or nitrogen bases, such as, for example, N,N-dimethylaniline.

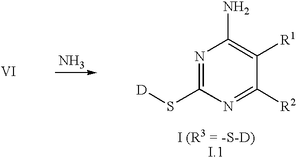

Using ammonia in inert solvents, the halogenation product VI is then, at 100° C. to 200° C., converted into the 4-aminopyrimidines I in which R³ is a group S-D (formula I.1). The reaction is preferably carried out using a 1- to 10-molar excess of ammonia under a pressure of from 1 to 100 bar.

Thioethers I.1 in which R³ is a group S-D can be oxidized to give the corresponding sulfoxides or sulfones I.1. The oxidation is preferably carried out at from 10 to 50° C. in the presence of protic or aproptic solvents [cf.: B. Kor. Chem. Soc., Vol. 16, pp. 489-492 (1995); Z. Chem., Vol. 17, p. 63 (1977)]. Suitable oxidizing agents are, for example, hydrogen peroxide or 3-chloroperbenzoic acid. Hydrogen peroxide and peracids of organic carboxylic acids have been found to be particularly suitable oxidizing agents. The oxidation can also be carried out using selenium dioxide [cf.: ref. WO 02/88127].

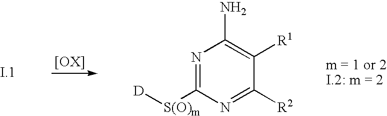

The compounds of the formula I.2 are useful intermediates for preparing further compounds I. For practical reasons, particular preference is given to compounds I.2 in which D is C₁-C₄-alkyl, in particular methyl. In formula I.2, the substituents R¹ and R² are as defined in formula I.

For preparing compounds of the formula I in which R³ is cyano or a group attached via a heteroatom, hydroxyl, mercapto, azido, alkoxy, alkenyloxy, alkynyloxy, haloalkoxy, alkylthio, alkenylthio, alkynylthio, haloalkylthio, —ON═CR^{a}R^{b}, —NR^{a}N═CR^{a}R^{b}, NR^{a}R^{b}, —NR^{a}NR^{a}R^{b} or —NOR^{a}, it is advantageous to use sulfones of the formula I.2 as starting materials.

The sulfones of the formula I.2 are reacted with compounds of the formula VII under basic conditions. For practical reasons, it is alternatively possible to employ directly the alkali metal, alkaline earth metal or ammonium salt of the compound VII.

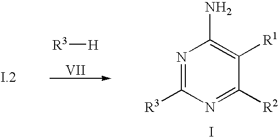

In the case of sufficiently nucleophilic reagents, this reaction is carried out under the conditions of nucleophilic substitution, usually at from 0 to 200° C., preferably from 10 to 150° C. in the presence of a dipolar aprotic solvent, such as N,N-dimethylformamide, tetrahydrofuran or acetonitrile [cf. DE-A 39 01 084; Chimia, Vol. 50, pp. 525-530 (1996); Khim. Geterotsikl. Soedin, Vol. 12, pp. 1696-1697 (1998)].

In general, the components are employed in approximately stoichiometric amounts. However, it may be advantageous to use an excess of the nucleophile of the formula R³—H.

In general, the reaction is carried out in the presence of a base which may be employed in equimolar amounts or else in excess. Suitable bases are alkali metal carbonates and bicarbonates, for example Na₂CO₃ and NaHCO₃, nitrogen bases, such as triethylamine, tributylamine and pyridine, alkali metal alkoxides, such as sodium ethoxide or potassium tert-butoxide, alkali metal amides, such as NaNH₂, or else alkali metal hydrides, such as LiH or NaH.

Suitable solvents are halogenated hydrocarbons, ethers, such as diethyl ether, diisopropyl ether, tert-butyl methyl ether, 1,2-dimethoxyethane, dioxane, anisole and tetrahydrofuran, and also dimethyl sulfoxide, dimethylformamide and dimethylacetamide. Particular preference is given to ethanol, dichloromethane, acetonitrile and tetrahydrofuran. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate. The bases are generally employed in catalytic amounts; however, they can also be used in excess.

The starting materials are generally reacted with one another in equimolar amounts. In terms of yield, it may be advantageous to employ an up to 10-fold, in particular up to 3-fold, excess of VII, based on I.2.

The compounds I in which R³ is cyano (formula I.3) are useful intermediates for preparing further compounds I.

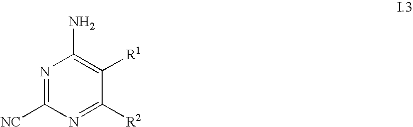

Compounds of the formula I in which R³ is hydrogen, alkyl, alkenyl, alkynyl or haloalkyl are advantageously obtained from β-ketoesters of the formula II by reaction with amidines of the formula IIIa.

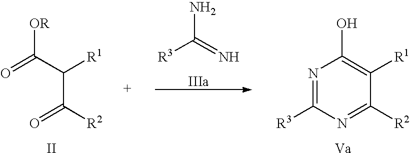

If, as amidine component IIIa, guanidine (R³═NH₂) is reacted with the β-ketoester II, 2-aminopyrimidines are obtained. Using generally customary alkylation and acylation processes, it is thus possible to synthesize, in a simple manner, pyrimidines according to the invention having, in the 2-position, a radical attached via nitrogen.

Analogously to the reaction sequence described above, the compounds Va are initially halogenated to give the 4-halopyrimidines VIa, which compounds VIa are reacted with ammonia under the conditions described for the compounds VI to give the corresponding compounds of the formula I.

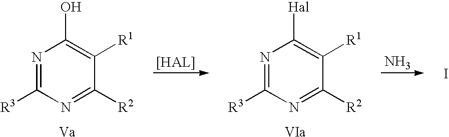

An advantageous route for preparing the pyrimidines I in which R³ is a group attached via nitrogen uses β-ketoesters II as starting materials. Reaction with urea IIIb gives the compounds Vb which can be halogenated, preferably chlorinated, to give VIb.

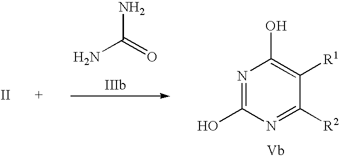

Using halogenating agents, in particular chlorinating agents or brominating agents, hydroxypyrimidines of the formula Vb are converted into halogen compounds of the formula VIb in which Hal is chlorine or bromine, in particular chlorine. Suitable chlorinating agents are, for example, POCl₃, PCl₃/Cl₂ or PCl₅, or mixtures of these reagents. The reaction can be carried out in excess chlorinating agent (POCl₃) or in an inert solvent, such as, for example, acetonitrile, toluene, chlorobenzene or 1,2-dichloroethane. Preference is given to carrying out the reaction in POCl₃ [cf. J. Chem. Soc. (1943) p. 383; Helv. Chim. Acta (1981) Vol. 64, pp. 113-152].

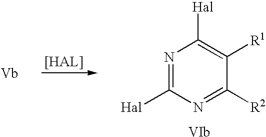

This reaction is usually carried out between 10 and 180° C. For practical reasons, the reaction temperature usually corresponds to the boiling point of the chlorinating agent (POCl₃) or solvent employed. The process is advantageously carried out with addition of catalytic or substoichiometric amounts of N,N-dimethylformamide or nitrogen bases, such as, for example, N,N-dimethylaniline.

By reaction with ammonia, VIb gives 2,4-diaminopyrimidines of the formula I in which R³ is NH₂.

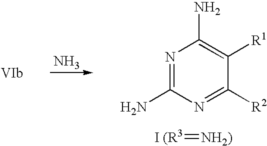

This reaction is usually carried out using ammonia in inerten solvents at from 100° C. to 200° C. The reaction is preferably carried out using a 1- to 10-molar excess of ammonia under a pressure of from 1 to 100 bar.

Using generally known alkylation or acylation methods, the 2-amino group in formula I can be converted into other groups R³ which are attached via nitrogen. Preferred alkylating or acylating agents are the alkylating agents D-X, such as dialkyl sulfate, alkyl halides, carbonyl chlorides, carboxylic anhydrides [cf.: Chem. Ber. Vol. 87, p. 1769 (1954)]

In the case of strong nucleophiles R³—H of the formula VII, the introduction of the substituent R³ into the nitrile of the formula I.3 is carried out under the conditions of nucleophilic substitution. Moreover, the introduction can also be carried out under transition metal catalysis, such as, for example, under the Suzuki coupling reaction conditions. This reaction is advantageously carried out under the conditions known from J. Chem. Soc. (1943) p. 388 and J. Org. Chem. (1952) Vol. 17, p. 1320.

Alternatively, compounds of the formula I can be obtained by reacting substituted acyl cyanides of the formula VIII in which R¹ and R² are as defined for formula I with thiourea of the formula III.

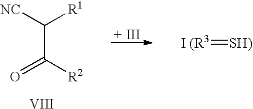

The reaction can be carried out in the presence or absence of solvents. It is advantageous to use solvents which are substantially inert toward the reactants and in which the reactants are completely or partially soluble. Suitable solvents are in particular alcohols, such as ethanol, propanols, butanols, glycols or glycol monoethers, diethylene glycols or monoethers thereof, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, as mentioned above, and mixtures of these solvents with water. The reaction temperatures are between 50 and 300° C., preferably from 50 to 150° C., if the reaction is carried out in solution.

Some of the substituted alkyl cyanides of the formula VIII required for preparing the compounds I are known, or they can be prepared by known methods from alkyl cyanides and carboxylic acid esters using strong bases, for example alkali metal hydrides, alkali metal alkoxides, alkali metal amides or metal alkyls (cf.: J. Amer. Chem. Soc. Vol. 73, (1951), p. 3766).

The further conversion of these thio compounds via alkylation and oxidation to give sulfoxides and sulfones which can be reated further with compounds R³—H of the formula VII has already been described further above.

Alternatively, compounds of the formula I in which R³ is NR^{a}CN can also be prepared from 5,6-dialkyl-7-aminotriazolopyrimidines of the formula IX which are reacted under basic conditions with alkylating agents of the formula VIIa.

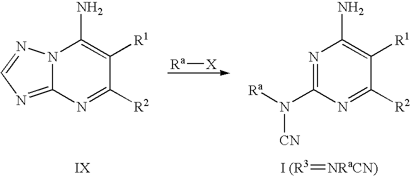

In formula VIIIa, X is a nucleophilically exchangeable group, such as a halogen atom, in particular an iodine atom. The reaction of VIIa with IX is usually carried out at temperatures of from −78° C. to 100° C., preferably from 10° C. to 80° C., in an inert organic solvent in the presence of a base [cf. WO 01/96314].

Compounds of the formula IX are known in a general manner from EP-A 141 317.

Compounds of the formula I in which R³ is a derivatized carboxylic acid group, such as C(═O)R^{c}, —C(═O)NR^{a}R^{b}, —C(═NOR^{c})NR^{a}R^{b}, —C(═NNR^{a}R^{b})R^{c} or —C(═NOR^{a})R^{c}, are advantageously obtained from compounds of the formula I.3.

Compounds of the formula I in which R³ is —C(═O)NR^{a}R^{b} or —C(═NOR^{c})NR^{a}R^{b} are obtainable from compounds of the formula I.3 by hydrolysis under acidic or basic conditions, to give the carboxylic acids of the formula I (where R³═COOH), and amidation with amines HNR^{a}R^{b}. The hydrolysis is usually carried out in inert polar solvents, such as water or alcohols, preferably using inorganic bases, such as alkali metal or alkaline earth metal hydroxides, in particular NaOH.

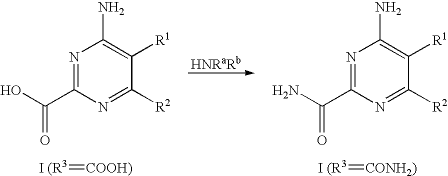

These reactions are advantageously carried out under the conditions known from Chem. and Pharm. Bull. 1982, Vol. 30, N12, p. 4314.

Amides of the formula I (where R³═CONH₂) afford, by oximation with substituted hydroxylamines H₂N—OR^{c} under basic conditions, the compounds of the formula I in which R³ is —C(═NOR^{c})NR^{a}R^{b} [cf. U.S. Pat. No. 4,876,252]. The substituted hydroxylamines can be employed as free base or, preferably, in the form of their acid addition salts. For practical reasons, the halides, such as the chlorides or the sulfates, are particularly suitable.

Alternatively, the amidoximes of the formula I in which R³ is —C(═NOR^{c})NH₂ can also be obtained form the corresponding nitriles of the formula I.3 by reaction with hydroxylamine and subsequent alkylation. This reaction is advantageously carried out under the conditions known from DE-A 198 37 794.

Compounds of the formula I in which R³ is —C(═O)R^{c} can be obtained from the corresponding nitriles of the formula I.3 by reaction with Grignard reagents R^{c}—Mg-Hal, where Hal is a halogen atom, in particular chlorine or bromine.

This reaction is advantageously carried out under the conditions known from J. Heterocycl. Chem. 1994, Vol. 31(4), p. 1041.

Compounds of the formula I in which R³ is —C(═NNR^{a}R^{b})R^{c} can be obtained via compounds I (where R³═C(O)R^{c}) which are reacted with hydrazines H₂NNR^{a}R^{b}, preferably under the conditions known from J. Org. Chem. 1966, Vol. 31, p. 677.

Compounds of the formula I in which R³ is —C(═NOR^{a})R^{c} can be obtained via oximation of compounds I (where R³═C(O)R^{c}). The oximation is carried out as described above.

Compounds of the formula I in which R¹ is C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-haloalkenyl or C₂-C₁₂-haloalkynyl can be obtained by halogenation of corresponding halogen-free pyrimidines of the formula I, they are referred to as compounds I′. In formula I′, R^{1′} is a halogen-free group R¹. In formula I″, R^{1″} is a halogenated group R¹:

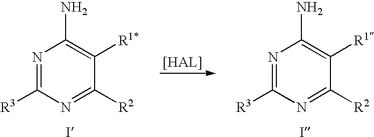

The halogenation is usually carried out at temperatures of from 0° C. to 200° C., preferably from 20° C. to 110° C., in an inert organic solvent in the presence of a free-radical initiator (for example dibenzoyl peroxide or azobisisobutyronitrile or under UV irradiation using, for example, an Hg vapor lamp) or an acid [cf. Synthetic Reagents, Vol. 2, pp. 1-63, Wiley Publishers, New York (1974)].

The reaction partners are generally reacted with one another in equimolar amounts. In terms of yield, it may be advantageous to employ an excess of halogenating agent, based on I′.

Suitable halogenating agents are, for example, elemental halogens (for example Cl₂, Br₂, I₂), N-bromosuccinimide, N-chlorosuccinimide oder dibromodimethylhydrantoin. The halogenating agents are generally employed in equimolar amounts, in excess or, if appropriate, as solvent.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required, however, since in some cases the individual isomers can be interconverted during work-up for use or during application (for example under the action of light, acids or bases). Such conversions may also take place after use, for example in the treatment of plants in the treated plants, or in the harmful fungus to be controlled.

In the definitions of symbols given in the formulae above, collective terms were used which are generally representative of the following substituents:

halogen: fluorine, chlorine, bromine and iodine;
alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where in these groups some or all of the hydrogen atoms may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
haloalkenyl: unsaturated straight-chain or branched hydrocarbon radicals having 2 to 10 carbon atoms and one or two double bonds in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- or six-membered saturated, partially unsaturated or aromatic heterocycle which contains one to four heteroatoms from the group consisting of O, N and S:
5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three and one to four nitrogen atoms, respectively, as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl;
   alkylene: divalent unbranched chains of 1 to 5 CH₂ groups, for example CH₂, CH₂CH₂, CH₂CH₂CH₂, CH₂CH₂CH₂CH₂ and CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

The scope of the present invention includes the (R) and (S) isomers and the racemates of compounds of the formula I having chiral centers.

With respect to the variables, the particularly preferred embodiments of the intermediates correspond to those of the formula I.

With a view to the intended use of the 4-aminopyrimidines of the formula I, particular preference is given to the following meanings of the substituents, in each case on their own or in combination:

Preference is given to compounds I in which the group R¹ has at most 9 carbon atoms. Preference is likewise given to compounds of the formula I in which the groups R¹ and R² together have at most 14 carbon atoms.

In one embodiment of the compounds I according to the invention, R¹ and R² independently of one another are halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₆-alkoxy-C₁-C₆-alkyl, where the carbon chains in R¹ and/or R² may be substituted by one to four identical or different of the groups R^{a} below:
halogen, cyano, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy-C₁-C₆-alkyl, phenyl which may be substituted by an alkyl group.

In a preferred embodiment of the compounds I according to the invention, R¹ and R² independently of one another are C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₆-alkoxy-C₁-C₆-alkyl, where the carbon chains in R¹ and/or R² may be substituted as described above.

Particular preference is given to those compounds I in which R² is C₁-C₅-alkyl, C₁-C₅-haloalkyl, C₂-C₅-alkenyl, C₂-C₅-alkynyl, C₃-C₅-cycloalkyl, C₁-C₅-alkoxy, C₁-C₄-alkoxy-C₁-C₄-alkyl, which groups are unsubstituted or substituted by halogen, cyano, methyl or ethyl.

In a further preferred embodiment of the compounds I according to the invention, R² is C₁-C₅-alkyl, C₁-C₅-haloalkyl, C₂-C₅-alkenyl, C₂-C₅-alkynyl, C₃-C₅-cycloalkyl, C₁-C₅-alkoxy, C₁-C₄-alkoxy-C₁-C₄-alkyl, which groups are unsubstituted or substituted by halogen, cyano, methyl or ethyl.

In a further particularly preferred embodiment of the compounds I according to the invention,
R¹ is C₁-C₁₂-alkyl, C₁-C₁₂-haloalkyl, C₂-C₁₂-alkenyl, C₃-C₁₂-alkynyl, C₁-C₆-alkoxy-C₁-C₆-alkyl, and
R² is C₁-C₅-alkyl, C₁-C₅-haloalkyl, C₂-C₅-alkenyl, C₂-C₅-alkynyl, C₁-C₄-alkoxy-C₁-C₄-alkyl,
   where the carbon chains in R¹ and/or R² may be partially or fully halogenated or substituted by C₂-C₅-alkenyl or C₂-C₅-alkynyl.

In a further particularly preferred embodiment of the compounds I according to the invention, R² is C₁-C₅-alkyl, C₁-C₅-haloalkyl, C₂-C₅-alkenyl, C₂-C₅-alkynyl, C₁-C₄-alkoxy-C₁-C₄-alkyl, where the carbon chains in R¹ and/or R² may be partially or fully halogenated.

In a preferred embodiment of the compounds of the formula I, group R¹ is absent.

In one embodiment of the compounds I according to the invention, R² is methyl, ethyl, isopropyl, n-propyl or n-butyl, in particular methyl.

Halogen atoms in the groups R¹ and/or R² are preferably located at the α or at the terminal carbon atom.

Cyano groups in R¹ and/or R² are preferably located at the terminal carbon atom.

In a further preferred embodiment of the compounds of the formula I, group R^{b} is absent.

In a further preferred embodiment of the compounds I according to the invention, R³ is halogen, cyano, hydroxyl, mercapto, amino, C₂-C₆-alkyl, C₁-C₆-haloalkyl, C₃-C₈-cycloalkyl, C₁-C₆-alkoxy or C₁-C₆-alkylthio.

Particular preference is likewise given to compounds I in which R³ is hydrogen, cyano, azido, C₁-C₆-alkyl, C₂-C₈-alkenyl, C₂-C₈-alkynyl, C₁-C₆-haloalkyl, or —ON═CR^{a}R^{b} or —NR^{c}N═CR^{a}R^{b} or —C(═NOR^{c})NR^{a}R^{b}.

Especially preferred are compounds I in which R³ is cyano, —CR^{a}NOR^{b} or —ON═CR^{a}R^{b}, in particular —ON═CR^{a}R^{b}.

In addition, preference is given to compounds I in which R³ is —NH(═NH)NHR^{c}, —NHC(═O)NHR^{a}, —NHC(═O)R^{a}, —OC(═O)R^{a}, —C(═NOR^{c})NH₂ or —CR^{c}(═NNR^{a}R^{b}).

Preference is furthermore given to compounds I in which R³ is —NR^{c}N═CR^{a}R^{b}.

Preference is likewise given to compounds I in which R³ is —C(═NOR^{c})NR^{a}R^{b}, in particular —C(═NOR^{c})NH₂.

In addition, particular preference is given to compounds I in which R³ is an aromatic five-membered heterocycle which is preferably attached via N and/or may be substituted by one or two groups R^{A}.

Preference is furthermore given to pyrimidines of the formula I in which R³ is cyano, CO₂R^{a}, C(═O)NR^{z}R^{b}, C(═NOR^{a})NR^{z}R^{b}, C(═NR^{a})NR^{z}R^{b}, C(═O)NR^{a}—NR^{z}R^{b}, C(═N—NR^{z}R^{c})NR^{a}R^{b}, C(═O)R^{a}, C(═NOR^{b})R^{a}, C(═O)—N(R^{a})—OR^{b}, C(═S)—NR^{a}R^{b}, C(═N—NR^{z}R^{b})R^{a}, CR^{a}R^{b}—OR^{z} or CR^{a}R^{b}—NR^{z}R^{c}.

Especially preferred are pyrimidines of the formula I in which R³ is cyano, C(═O)NR^{z}R^{b}, C(═O)—N(R^{a})—OR^{b}, C(═S)—NR^{a}R^{b}, C(═NOR^{a})NR^{z}R^{b}, C(═NOR^{b})R^{a}, C(═N—NR^{z}R^{b})R^{a} or CR^{a}R^{b}—NR^{z}R^{c}.

Moreover, preference is given to pyrimidines of the formula I in which R³ is ON(═CR^{a}R^{b}) or O—C(═O)R^{a}.

Preference is furthermore given to pyrimidines of the formula I in which R³ is NR^{a}R^{b′}, NR^{a}(C(═O)R^{b}), NR^{a}(C(═O)OR^{b}), NR^{a}(C(═O)—NR^{z}R^{b}), NR^{a}(C(═NR^{c})R^{b}), NR^{a}(N═CR^{c}R^{b}), NR^{a}—NR^{z}R^{b}, NR^{z}—OR^{a}, NR^{a}(C(═NR^{c})—NR^{z}R^{b}), NR^{a}(C(═NOR^{c})R^{b}).

Especially preferred are pyrimidines of the formula I in which R³ is NR^{a}(C(═O)R^{b}), NR^{a}(C(═O)OR^{b}), NR^{a}(N═CR^{c}R^{b}), NR^{z}—OR^{a}.

R^{a}, R^{b} and R^{c} are preferably independently of one another hydrogen, C₁-C₆-alkyl, C₂-C₆-alkenyl, C₂-C₆-alkynyl or C₃-C₆-cycloalkyl.

R^{z} has preferably the abovementioned preferred meanings of R^{a}, R^{b} and R^{c}. Particularly preferred is the meaning —CO—R^{a}.

Especially preferred are the following groups of compounds of the formula I:

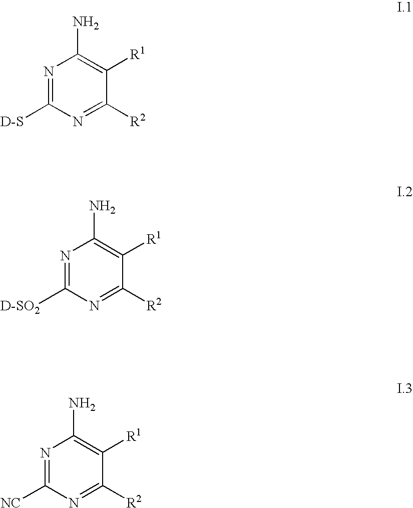

If compounds I.1 and I.2 are used as intermediates, D is in particular C₁-C₄-alkyl, preferably methyl.

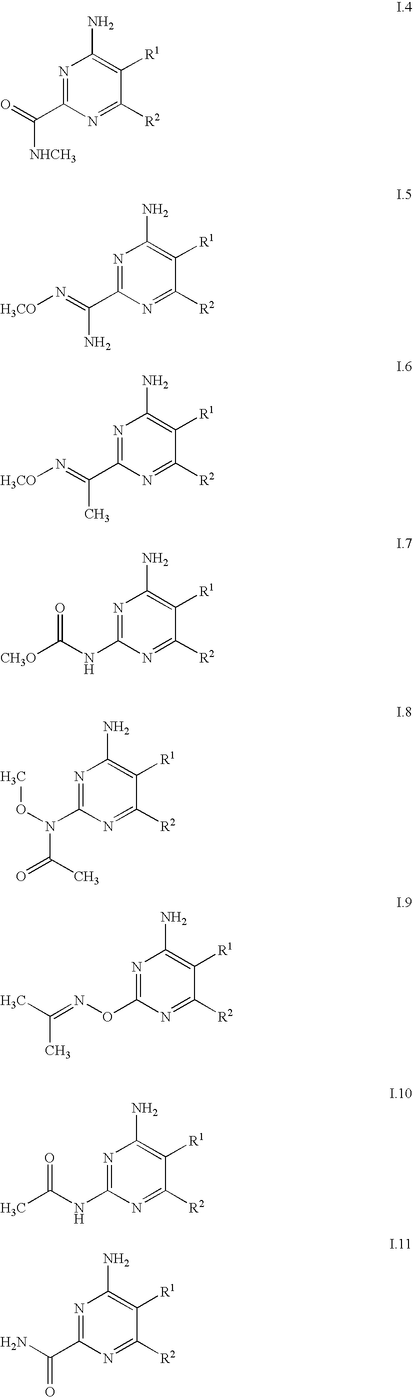

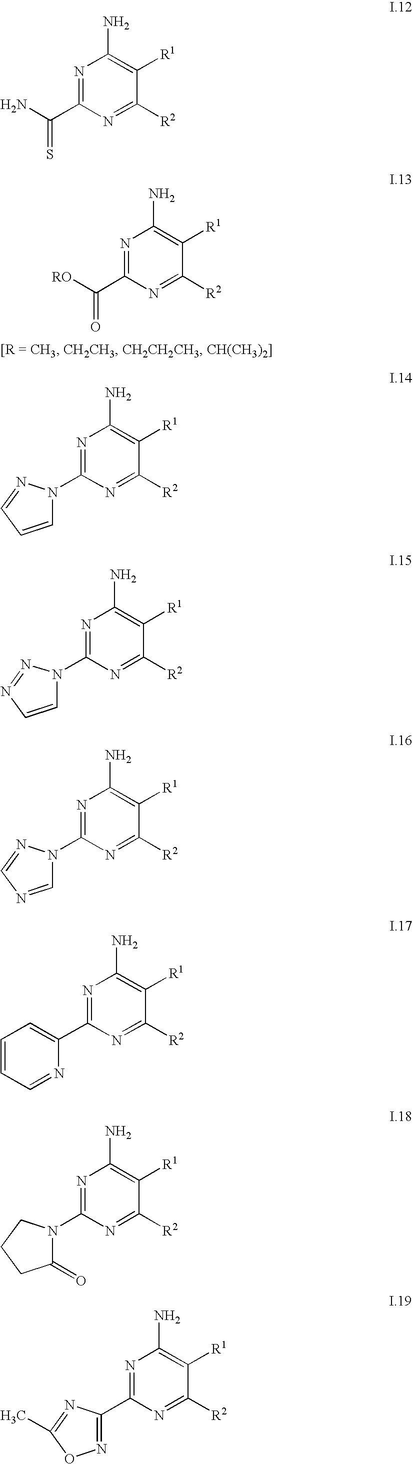

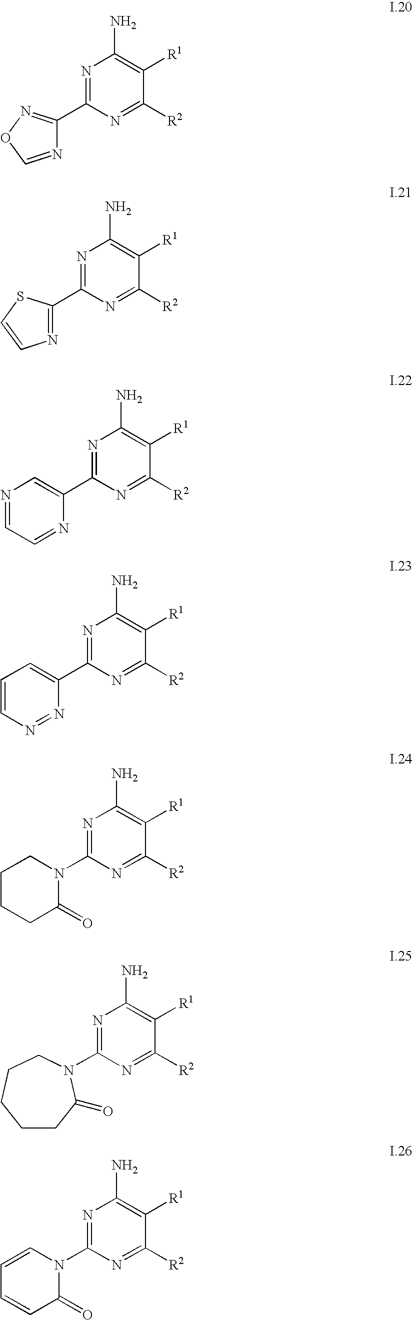

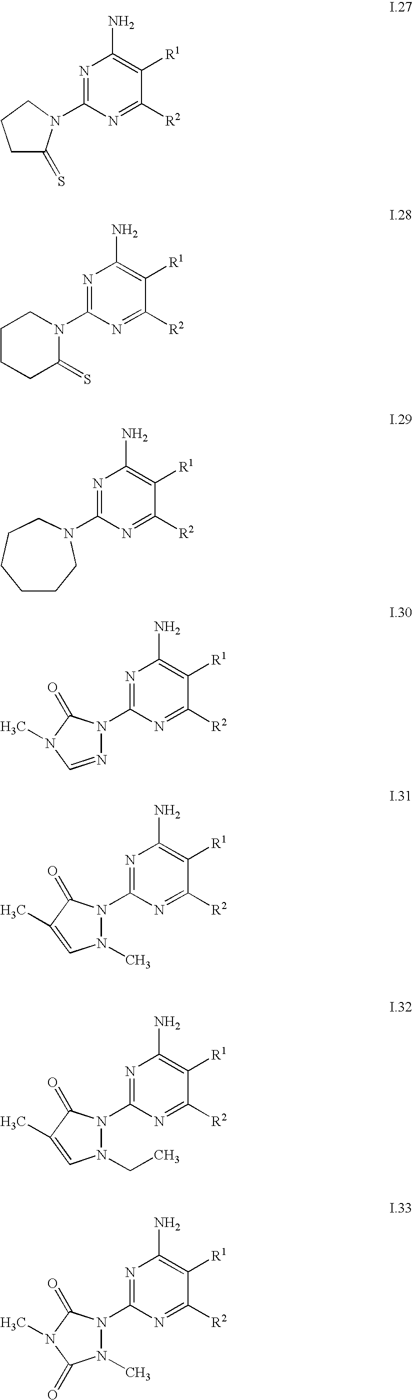

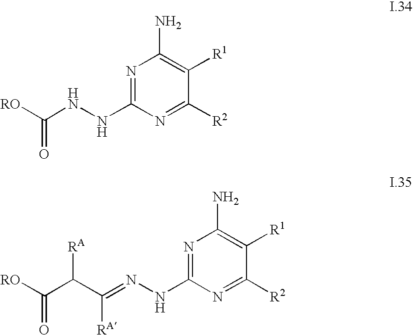

Compounds of the formulae I.34 and I.35 are also useful intermediates for preparing other compounds I. In the formulae I.34 and I.35, R is C₁-C₄-alkyl, in particular methyl, and R^{A} and R^{A′} are in particular methyl.

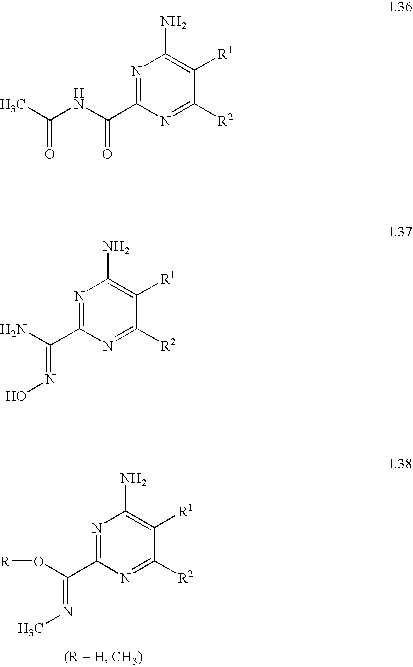

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula I.1 in which R¹ for each compound corresponds to one row of Table A, D is methyl and R² is methyl

### Table 2

Compounds of the formula I.2 in which R¹ for each compound corresponds to one row of Table A, D is methyl and R² is methyl

### Table 3

Compounds of the formula I.3 in which R¹ for each compound corresponds to one row of Table A and R² is methyl

### Table 4

Compounds of the formula I.4 in which R¹ for each compound corresponds to one row of Table A and R² is methyl

### Table 5

Compounds of the formula I.5 in which R¹ for each compound corresponds to one row of Table A and R² is methyl

### Table 6

Compounds of the formula I.6 in which R¹ for each compound corresponds to one row of Table A and R² is methyl

### Table 7

Compounds of the formula I.7 in which R¹ for each compound corresponds to one row of Table A and R² is methyl

### Table 8

Compounds of the formula I.8 in which R¹ for each compound corresponds to one row of Table A and R² is methyl

### Table 9

Compounds of the formula I.9 in which R¹ for each compound corresponds to one row of Table A and R² is methyl

### Table 10

Compounds of the formula I.10 in which R¹ for each compound corresponds to one row of Table A and R² is methyl

### Table 11

Compounds of the formula I.11 in which R¹ for each compound corresponds to one row of Table A and R² is methyl

### Table 12

Compounds of the formula I.12 in which R¹ for each compound corresponds to one row of Table A and R² is methyl

### Table 13

Compounds of the formula I.13 in which R¹ for each compound corresponds to one row of Table A and R² and R are methyl

### Table 14

Compounds of the formula I.13 in which R¹ for each compound corresponds to one row of Table A, R² is methyl and R is ethyl

### Table 15

Compounds of the formula I.13 in which R¹ for each compound corresponds to one row of Table A, R² is methyl and R is n-propyl

### Table 16

Compounds of the formula I.13 in which R¹ for each compound corresponds to one row of Table A, R² is methyl and R is isopropyl

### Table 17

Compounds of the formula I.14 in which R¹ for each compound corresponds to one row of Table A and R² is methyl

### Table 18

Compounds of the formula I.15 in which R¹ for each compound corresponds to one row of Table A and R² is methyl

### Table 19

Compounds of the formula I.16 in which R¹ for each compound corresponds to one row of Table A and R² is methyl

### Table 20

Compounds of the formula I.17 in which R¹ for each compound corresponds to one row of Table A and R² is methyl

### Table 21

Compounds of the formula I.18 in which R¹ for each compound corresponds to one row of Table A and R² is methyl

### Table 22

Compounds of the formula I.19 in which R¹ for each compound corresponds to one row of Table A and R² is methyl

### Table 23

Compounds of the formula I.20 in which R¹ for each compound corresponds to one row of Table A and R² is methyl

### Table 24

Compounds of the formula I.21 in which R¹ for each compound corresponds to one row of Table A and R² is methyl

### Table 25

Compounds of the formula I.22 in which R¹ for each compound corresponds to one row of Table A and R² is methyl

### Table 26

Compounds of the formula I.23 in which R¹ for each compound corresponds to one row of Table A and R² is methyl

### Table 27

Compounds of the formula I.24 in which R¹ for each compound corresponds to one row of Table A and R² is methyl

### Table 28

Compounds of the formula I.25 in which R¹ for each compound corresponds to one row of Table A and R² is methyl

### Table 29

Compounds of the formula I.26 in which R¹ for each compound corresponds to one row of Table A and R² is methyl

### Table 30

Compounds of the formula I.27 in which R¹ for each compound corresponds to one row of Table A and R² is methyl

### Table 31

Compounds of the formula I.28 in which R¹ for each compound corresponds to one row of Table A and R² is methyl

### Table 32

Compounds of the formula I.29 in which R¹ for each compound corresponds to one row of Table A and R² is methyl

### Table 33

Compounds of the formula I.30 in which R¹ for each compound corresponds to one row of Table A and R² is methyl

### Table 34

Compounds of the formula I.31 in which R¹ for each compound corresponds to one row of Table A and R² is methyl

### Table 35

Compounds of the formula I.32 in which R¹ for each compound corresponds to one row of Table A and R² is methyl

### Table 36

Compounds of the formula I.33 in which R¹ for each compound corresponds to one row of Table A and R² is methyl

### Table 37

Compounds of the formula I.34 in which R¹ for each compound corresponds to one row of Table A and R² and R are methyl

### Table 38

Compounds of the formula I.35 in which R¹ for each compound corresponds to one row of Table A and R, R², R^{A and R}^{A′} are methyl

### Table 39

Compounds of the formula I.36 in which R¹ for each compound corresponds to one row of Table A and R² is methyl

### Table 40

Compounds of the formula I.37 in which R¹ for each compound corresponds to one row of Table A and R² is methyl

### Table 41

Compounds of the formula I.38 in which R¹ for each compound corresponds to one row of Table A, R² is methyl and R is hydrogen

### Table 42

Compounds of the formula I.38 in which R¹ for each compound corresponds to one row of Table A and R² and R are methyl

### Table 43

Compounds of the formula I.1 in which R¹ for each compound corresponds to one row of Table A, D is methyl and R² is ethyl

### Table 44

Compounds of the formula I.2 in which R¹ for each compound corresponds to one row of Table A, D is methyl and R² is ethyl

### Table 45

Compounds of the formula I.3 in which R¹ for each compound corresponds to one row of Table A and R² is ethyl

### Table 46

Compounds of the formula I.4 in which R¹ for each compound corresponds to one row of Table A and R² is ethyl

### Table 47

Compounds of the formula I.5 in which R¹ for each compound corresponds to one row of Table A and R² is ethyl

### Table 48

Compounds of the formula I.6 in which R¹ for each compound corresponds to one row of Table A and R² is ethyl

### Table 49

Compounds of the formula I.7 in which R¹ for each compound corresponds to one row of Table A and R² is ethyl

### Table 50

Compounds of the formula I.8 in which R¹ for each compound corresponds to one row of Table A and R² is ethyl

### Table 51

Compounds of the formula I.9 in which R¹ for each compound corresponds to one row of Table A and R² is ethyl

### Table 52

Compounds of the formula I.10 in which R¹ for each compound corresponds to one row of Table A and R² is ethyl

### Table 53

Compounds of the formula I.11 in which R¹ for each compound corresponds to one row of Table A and R² is ethyl

### Table 54

Compounds of the formula I.12 in which R¹ for each compound corresponds to one row of Table A and R² is ethyl

### Table 55

Compounds of the formula I.13 in which R¹ for each compound corresponds to one row of Table A, R² is ethyl and R is methyl

### Table 56

Compounds of the formula I.13 in which R¹ for each compound corresponds to one row of Table A and R² and R are ethyl

### Table 57

Compounds of the formula I.13 in which R¹ for each compound corresponds to one row of Table A, R² is ethyl and R is n-propyl

### Table 58

Compounds of the formula I.13 in which R¹ for each compound corresponds to one row of Table A, R² is ethyl and R is isopropyl

### Table 59

Compounds of the formula I.14 in which R¹ for each compound corresponds to one row of Table A and R² is ethyl

### Table 60

Compounds of the formula I.15 in which R¹ for each compound corresponds to one row of Table A and R² is ethyl

### Table 61

Compounds of the formula I.16 in which R¹ for each compound corresponds to one row of Table A and R² is ethyl

### Table 62

Compounds of the formula I.17 in which R¹ for each compound corresponds to one row of Table A and R² is ethyl

### Table 63

Compounds of the formula I.18 in which R¹ for each compound corresponds to one row of Table A and R² is ethyl

### Table 64

Compounds of the formula I.19 in which R¹ for each compound corresponds to one row of Table A and R² is ethyl

### Table 65

Compounds of the formula I.20 in which R¹ for each compound corresponds to one row of Table A and R² is ethyl

### Table 66

Compounds of the formula I.21 in which R¹ for each compound corresponds to one row of Table A and R² is ethyl

### Table 67

Compounds of the formula I.22 in which R¹ for each compound corresponds to one row of Table A and R² is ethyl

### Table 68

Compounds of the formula I.23 in which R¹ for each compound corresponds to one row of Table A and R² is ethyl

### Table 69

Compounds of the formula I.24 in which R¹ for each compound corresponds to one row of Table A and R² is ethyl

### Table 70

Compounds of the formula I.25 in which R¹ for each compound corresponds to one row of Table A and R² is ethyl

### Table 71

Compounds of the formula I.26 in which R¹ for each compound corresponds to one row of Table A and R² is ethyl

### Table 72

Compounds of the formula I.27 in which R¹ for each compound corresponds to one row of Table A and R² is ethyl

### Table 73

Compounds of the formula I.28 in which R¹ for each compound corresponds to one row of Table A and R² is ethyl

### Table 74

Compounds of the formula I.29 in which R¹ for each compound corresponds to one row of Table A and R² is ethyl

### Table 75

Compounds of the formula I.30 in which R¹ for each compound corresponds to one row of Table A and R² is ethyl

### Table 76

Compounds of the formula I.31 in which R¹ for each compound corresponds to one row of Table A and R² is ethyl

### Table 77

Compounds of the formula I.32 in which R¹ for each compound corresponds to one row of Table A and R² is ethyl

### Table 78

Compounds of the formula I.33 in which R¹ for each compound corresponds to one row of Table A and R² is ethyl

### Table 79

Compounds of the formula I.34 in which R¹ for each compound corresponds to one row of Table A, R² is ethyl and R is methyl

### Table 80

Compounds of the formula I.35 in which R¹ for each compound corresponds to one row of Table A, R² is ethyl and R, R^{A} and R^{A′} are methyl

### Table 81

Compounds of the formula I.36 in which R¹ for each compound corresponds to one row of Table A and R² is ethyl

### Table 82

Compounds of the formula I.37 in which R¹ for each compound corresponds to one row of Table A and R² is ethyl

### Table 83

Compounds of the formula I.38 in which R¹ for each compound corresponds to one row of Table A, R² is ethyl and R is hydrogen

### Table 84

Compounds of the formula I.38 in which R¹ for each compound corresponds to one row of Table A, R² is ethyl and R is methyl

### Table 85

Compounds of the formula I.1 in which R¹ for each compound corresponds to one row of Table A, D is methyl and R² is n-propyl

### Table 86

Compounds of the formula I.2 in which R¹ for each compound corresponds to one row of Table A, D is methyl and R² is n-propyl

### Table 87

Compounds of the formula I.3 in which R¹ for each compound corresponds to one row of Table A and R² is n-propyl

### Table 88

Compounds of the formula I.4 in which R¹ for each compound corresponds to one row of Table A and R² is n-propyl

### Table 89

Compounds of the formula I.5 in which R¹ for each compound corresponds to one row of Table A and R² is n-propyl

### Table 90

Compounds of the formula I.6 in which R¹ for each compound corresponds to one row of Table A and R² is n-propyl

### Table 91

Compounds of the formula I.7 in which R¹ for each compound corresponds to one row of Table A and R² is n-propyl

### Table 92

Compounds of the formula I.8 in which R¹ for each compound corresponds to one row of Table A and R² is n-propyl

### Table 93

Compounds of the formula I.9 in which R¹ for each compound corresponds to one row of Table A and R² is n-propyl

### Table 94

Compounds of the formula I.10 in which R¹ for each compound corresponds to one row of Table A and R² is n-propyl

### Table 95

Compounds of the formula I.11 in which R¹ for each compound corresponds to one row of Table A and R² is n-propyl

### Table 96

Compounds of the formula I.12 in which R¹ for each compound corresponds to one row of Table A and R² is n-propyl

### Table 97

Compounds of the formula I.13 in which R¹ for each compound corresponds to one row of Table A, R² is n-propyl and R is methyl

### Table 98

Compounds of the formula I.13 in which R¹ for each compound corresponds to one row of Table A, R² is n-propyl and R is ethyl

### Table 99

Compounds of the formula I.13 in which R¹ for each compound corresponds to one row of Table A and R² and R are n-propyl

### Table 100

Compounds of the formula I.13 in which R¹ for each compound corresponds to one row of Table A, R² is n-propyl and R is isopropyl

### Table 101

Compounds of the formula I.14 in which R¹ for each compound corresponds to one row of Table A and R² is n-propyl

### Table 102

Compounds of the formula I.15 in which R¹ for each compound corresponds to one row of Table A and R² is n-propyl

### Table 103

Compounds of the formula I.16 in which R¹ for each compound corresponds to one row of Table A and R² is n-propyl

### Table 104

Compounds of the formula I.17 in which R¹ for each compound corresponds to one row of Table A and R² is n-propyl

### Table 105

Compounds of the formula I.18 in which R¹ for each compound corresponds to one row of Table A and R² is n-propyl

### Table 106

Compounds of the formula I.19 in which R¹ for each compound corresponds to one row of Table A and R² is n-propyl

### Table 107

Compounds of the formula I.20 in which R¹ for each compound corresponds to one row of Table A and R² is n-propyl

### Table 108

Compounds of the formula I.21 in which R¹ for each compound corresponds to one row of Table A and R² is n-propyl

### Table 109

Compounds of the formula I.22 in which R¹ for each compound corresponds to one row of Table A and R² is n-propyl

### Table 110

Compounds of the formula I.23 in which R¹ for each compound corresponds to one row of Table A and R² is n-propyl

### Table 111

Compounds of the formula I.24 in which R¹ for each compound corresponds to one row of Table A and R² is n-propyl

### Table 112

Compounds of the formula I.25 in which R¹ for each compound corresponds to one row of Table A and R² is n-propyl

### Table 113

Compounds of the formula I.26 in which R¹ for each compound corresponds to one row of Table A and R² is n-propyl

### Table 114

Compounds of the formula I.27 in which R¹ for each compound corresponds to one row of Table A and R² is n-propyl

### Table 115

Compounds of the formula I.28 in which R¹ for each compound corresponds to one row of Table A and R² is n-propyl

### Table 116

Compounds of the formula I.29 in which R¹ for each compound corresponds to one row of Table A and R² is n-propyl

### Table 117

Compounds of the formula I.30 in which R¹ for each compound corresponds to one row of Table A and R² is n-propyl

### Table 118

Compounds of the formula I.31 in which R¹ for each compound corresponds to one row of Table A and R² is n-propyl

### Table 119

Compounds of the formula I.32 in which R¹ for each compound corresponds to one row of Table A and R² is n-propyl

### Table 120

Compounds of the formula I.33 in which R¹ for each compound corresponds to one row of Table A and R² is n-propyl

### Table 121

Compounds of the formula I.34 in which R¹ for each compound corresponds to one row of Table A, R² is n-propyl and R is methyl

### Table 122

Compounds of the formula I.35 in which R¹ for each compound corresponds to one row of Table A, R² is n-propyl and R, R^{A} and R^{A′} are methyl

### Table 123

Compounds of the formula I.36 in which R¹ for each compound corresponds to one row of Table A and R² is n-propyl

### Table 124

Compounds of the formula I.37 in which R¹ for each compound corresponds to one row of Table A and R² is n-propyl

### Table 125

Compounds of the formula I.38 in which R¹ for each compound corresponds to one row of Table A, R² is n-propyl and R is hydrogen

### Table 126

Compounds of the formula I.38 in which R¹ for each compound corresponds to one row of Table A, R² is n-propyl and R is methyl

### Table 127

Compounds of the formula I.1 in which R¹ for each compound corresponds to one row of Table A, D is methyl and R² is isopropyl

### Table 128

Compounds of the formula I.2 in which R¹ for each compound corresponds to one row of Table A, D is methyl and R² is isopropyl

### Table 129

Compounds of the formula I.3 in which R¹ for each compound corresponds to one row of Table A and R² is isopropyl

### Table 130

Compounds of the formula I.4 in which R¹ for each compound corresponds to one row of Table A and R² is isopropyl

### Table 131

Compounds of the formula I.5 in which R¹ for each compound corresponds to one row of Table A and R² is isopropyl

### Table 132

Compounds of the formula I.6 in which R¹ for each compound corresponds to one row of Table A and R² is isopropyl

### Table 133

Compounds of the formula I.7 in which R¹ for each compound corresponds to one row of Table A and R² is isopropyl

### Table 134

Compounds of the formula I.8 in which R¹ for each compound corresponds to one row of Table A and R² is isopropyl

### Table 135

Compounds of the formula I.9 in which R¹ for each compound corresponds to one row of Table A and R² is isopropyl

### Table 136

Compounds of the formula I.10 in which R¹ for each compound corresponds to one row of Table A and R² is isopropyl

### Table 137

Compounds of the formula I.11 in which R¹ for each compound corresponds to one row of Table A and R² is isopropyl

### Table 138

Compounds of the formula I.12 in which R¹ for each compound corresponds to one row of Table A and R² is isopropyl

### Table 139

Compounds of the formula I.13 in which R¹ for each compound corresponds to one row of Table A, R² is isopropyl and R is methyl

### Table 140

Compounds of the formula I.13 in which R¹ for each compound corresponds to one row of Table A, R² is isopropyl and R is ethyl

### Table 141

Compounds of the formula I.13 in which R¹ for each compound corresponds to one row of Table A, R² is isopropyl and R is n-propyl

### Table 142

Compounds of the formula I.13 in which R¹ for each compound corresponds to one row of Table A and R² and R are isopropyl

### Table 143

Compounds of the formula I.14 in which R¹ for each compound corresponds to one row of Table A and R² is isopropyl

### Table 144

Compounds of the formula I.15 in which R¹ for each compound corresponds to one row of Table A and R² is isopropyl

### Table 145

Compounds of the formula I.16 in which R¹ for each compound corresponds to one row of Table A and R² is isopropyl

### Table 146

Compounds of the formula I.17 in which R¹ for each compound corresponds to one row of Table A and R² is isopropyl

### Table 147

Compounds of the formula I.18 in which R¹ for each compound corresponds to one row of Table A and R² is isopropyl

### Table 148

Compounds of the formula I.19 in which R¹ for each compound corresponds to one row of Table A and R² is isopropyl

### Table 149

Compounds of the formula I.20 in which R¹ for each compound corresponds to one row of Table A and R² is isopropyl

### Table 150

Compounds of the formula I.21 in which R¹ for each compound corresponds to one row of Table A and R² is isopropyl

### Table 151

Compounds of the formula I.22 in which R¹ for each compound corresponds to one row of Table A and R² is isopropyl

### Table 152

Compounds of the formula I.23 in which R¹ for each compound corresponds to one row of Table A and R² is isopropyl

### Table 153

Compounds of the formula I.24 in which R¹ for each compound corresponds to one row of Table A and R² is isopropyl

### Table 154

Compounds of the formula I.25 in which R¹ for each compound corresponds to one row of Table A and R² is isopropyl

### Table 155

Compounds of the formula I.26 in which R¹ for each compound corresponds to one row of Table A and R² is isopropyl

### Table 156

Compounds of the formula I.27 in which R¹ for each compound corresponds to one row of Table A and R² is isopropyl

### Table 157

Compounds of the formula I.28 in which R¹ for each compound corresponds to one row of Table A and R² is isopropyl

### Table 158

Compounds of the formula I.29 in which R¹ for each compound corresponds to one row of Table A and R² is isopropyl

### Table 159

Compounds of the formula I.30 in which R¹ for each compound corresponds to one row of Table A and R² is isopropyl

### Table 160

Compounds of the formula I.31 in which R¹ for each compound corresponds to one row of Table A and R² is isopropyl

### Table 161

Compounds of the formula I.32 in which R¹ for each compound corresponds to one row of Table A and R² is isopropyl

### Table 162

Compounds of the formula I.33 in which R¹ for each compound corresponds to one row of Table A and R² is isopropyl

### Table 163

Compounds of the formula I.34 in which R¹ for each compound corresponds to one row of Table A, R² is isopropyl and R is methyl

### Table 164

Compounds of the formula I.35 in which R¹ for each compound corresponds to one row of Table A, R² is isopropyl and R, R^{A} and R^{A′} are methyl

### Table 165

Compounds of the formula I.36 in which R¹ for each compound corresponds to one row of Table A and R² is isopropyl

### Table 166

Compounds of the formula I.37 in which R¹ for each compound corresponds to one row of Table A and R² is isopropyl

### Table 167

Compounds of the formula I.38 in which R¹ for each compound corresponds to one row of Table A, R² is isopropyl and R is hydrogen

### Table 168

Compounds of the formula I.38 in which R¹ for each compound corresponds to one row of Table A, R² is isopropyl and R is methyl

### Table 169

Compounds of the formula I.1 in which R¹ for each compound corresponds to one row of Table A, D is methyl and R² is n-butyl

### Table 170

Compounds of the formula I.2 in which R¹ for each compound corresponds to one row of Table A, D is methyl and R² is n-butyl

### Table 171

Compounds of the formula I.3 in which R¹ for each compound corresponds to one row of Table A and R² is n-butyl

### Table 172

Compounds of the formula I.4 in which R¹ for each compound corresponds to one row of Table A and R² is n-butyl

### Table 173

Compounds of the formula I.5 in which R¹ for each compound corresponds to one row of Table A and R² is n-butyl

### Table 174

Compounds of the formula I.6 in which R¹ for each compound corresponds to one row of Table A and R² is n-butyl

### Table 175

Compounds of the formula I.7 in which R¹ for each compound corresponds to one row of Table A and R² is n-butyl

### Table 176

Compounds of the formula I.8 in which R¹ for each compound corresponds to one row of Table A and R² is n-butyl

### Table 177

Compounds of the formula I.9 in which R¹ for each compound corresponds to one row of Table A and R² is n-butyl

### Table 178

Compounds of the formula I.10 in which R¹ for each compound corresponds to one row of Table A and R² is n-butyl

### Table 179

Compounds of the formula I.11 in which R¹ for each compound corresponds to one row of Table A and R² is n-butyl

### Table 180

Compounds of the formula I.12 in which R¹ for each compound corresponds to one row of Table A and R² is n-butyl

### Table 181

Compounds of the formula I.13 in which R¹ for each compound corresponds to one row of Table A, R² is n-butyl and R is methyl

### Table 182

Compounds of the formula I.13 in which R¹ for each compound corresponds to one row of Table A, R² is n-butyl and R is ethyl

### Table 183

Compounds of the formula I.13 in which R¹ for each compound corresponds to one row of Table A, R² is n-butyl and R is n-propyl

### Table 184

Compounds of the formula I.13 in which R¹ for each compound corresponds to one row of Table A, R² is n-butyl and R is isopropyl

### Table 185

Compounds of the formula I.14 in which R¹ for each compound corresponds to one row of Table A and R² is n-butyl

### Table 186

Compounds of the formula I.15 in which R¹ for each compound corresponds to one row of Table A and R² is n-butyl

### Table 187

Compounds of the formula I.16 in which R¹ for each compound corresponds to one row of Table A and R² is n-butyl

### Table 188

Compounds of the formula I.17 in which R¹ for each compound corresponds to one row of Table A and R² is n-butyl

### Table 189

Compounds of the formula I.18 in which R¹ for each compound corresponds to one row of Table A and R² is n-butyl

### Table 190

Compounds of the formula I.19 in which R¹ for each compound corresponds to one row of Table A and R² is n-butyl

### Table 191

Compounds of the formula I.20 in which R¹ for each compound corresponds to one row of Table A and R² is n-butyl

### Table 192

Compounds of the formula I.21 in which R¹ for each compound corresponds to one row of Table A and R² is n-butyl

### Table 193

Compounds of the formula I.22 in which R¹ for each compound corresponds to one row of Table A and R² is n-butyl

### Table 194

Compounds of the formula I.23 in which R¹ for each compound corresponds to one row of Table A and R² is n-butyl

### Table 195

Compounds of the formula I.24 in which R¹ for each compound corresponds to one row of Table A and R² is n-butyl

### Table 196

Compounds of the formula I.25 in which R¹ for each compound corresponds to one row of Table A and R² is n-butyl

### Table 197

Compounds of the formula I.26 in which R¹ for each compound corresponds to one row of Table A and R² is n-butyl

### Table 198

Compounds of the formula I.27 in which R¹ for each compound corresponds to one row of Table A and R² is n-butyl

### Table 199

Compounds of the formula I.28 in which R¹ for each compound corresponds to one row of Table A and R² is n-butyl

### Table 200

Compounds of the formula I.29 in which R¹ for each compound corresponds to one row of Table A and R² is n-butyl

### Table 201

Compounds of the formula I.30 in which R¹ for each compound corresponds to one row of Table A and R² is n-butyl

### Table 202

Compounds of the formula I.31 in which R¹ for each compound corresponds to one row of Table A and R² is n-butyl

Table 203

Compounds of the formula I.32 in which R¹ for each compound corresponds to one row of Table A and R² is n-butyl

### Table 204

Compounds of the formula I.33 in which R¹ for each compound corresponds to one row of Table A and R² is n-butyl

### Table 205

Compounds of the formula I.34 in which R¹ for each compound corresponds to one row of Table A, R² is n-butyl and R is methyl

### Table 206

Compounds of the formula I.35 in which R¹ for each compound corresponds to one row of Table A, R² is n-butyl and R, R^{A} and R^{A′} are methyl

### Table 207

Compounds of the formula I.36 in which R¹ for each compound corresponds to one row of Table A and R² is n-butyl

### Table 208

Compounds of the formula I.37 in which R¹ for each compound corresponds to one row of Table A and R² is n-butyl

### Table 209

Compounds of the formula I.38 in which R¹ for each compound corresponds to one row of Table A, R² is n-butyl and R is hydrogen

### Table 210

Compounds of the formula I.38 in which R¹ for each compound corresponds to one row of Table A, R² is n-butyl and R is methyl

### Table 211

Compounds of the formula I.1 in which R¹ for each compound corresponds to one row of Table A, D is methyl and R² is n-pentyl

### Table 212

Compounds of the formula I.2 in which R¹ for each compound corresponds to one row of Table A, D is methyl and R² is n-pentyl

### Table 213

Compounds of the formula I.3 in which R¹ for each compound corresponds to one row of Table A and R² is n-pentyl

### Table 214

Compounds of the formula I.4 in which R¹ for each compound corresponds to one row of Table A and R² is n-pentyl

### Table 215

Compounds of the formula I.5 in which R¹ for each compound corresponds to one row of Table A and R² is n-pentyl

### Table 216

Compounds of the formula I.6 in which R¹ for each compound corresponds to one row of Table A and R² is n-pentyl

### Table 217

Compounds of the formula I.7 in which R¹ for each compound corresponds to one row of Table A and R² is n-pentyl

### Table 218

Compounds of the formula I.8 in which R¹ for each compound corresponds to one row of Table A and R² is n-pentyl

### Table 219

Compounds of the formula I.9 in which R¹ for each compound corresponds to one row of Table A and R² is n-pentyl

### Table 220

Compounds of the formula I.10 in which R¹ for each compound corresponds to one row of Table A and R² is n-pentyl

### Table 221

Compounds of the formula I.11 in which R¹ for each compound corresponds to one row of Table A and R² is n-pentyl

### Table 222

Compounds of the formula I.12 in which R¹ for each compound corresponds to one row of Table A and R² is n-pentyl

### Table 223

Compounds of the formula I.13 in which R¹ for each compound corresponds to one row of Table A, R² is n-pentyl and R is methyl

### Table 224

Compounds of the formula I.13 in which R¹ for each compound corresponds to one row of Table A, R² is n-pentyl and R is ethyl

### Table 225

Compounds of the formula I.13 in which R¹ for each compound corresponds to one row of Table A, R² is n-pentyl and R is n-propyl

### Table 226

Compounds of the formula I.13 in which R¹ for each compound corresponds to one row of Table A, R² is n-pentyl and R is isopropyl

### Table 227

Compounds of the formula I.14 in which R¹ for each compound corresponds to one row of Table A and R² is n-pentyl

### Table 228

Compounds of the formula I.15 in which R¹ for each compound corresponds to one row of Table A and R² is n-pentyl

### Table 229

Compounds of the formula I.16 in which R¹ for each compound corresponds to one row of Table A and R² is n-pentyl

### Table 230

Compounds of the formula I.17 in which R¹ for each compound corresponds to one row of Table A and R² is n-pentyl

### Table 231

Compounds of the formula I.18 in which R¹ for each compound corresponds to one row of Table A and R² is n-pentyl

### Table 232

Compounds of the formula I.19 in which R¹ for each compound corresponds to one row of Table A and R² is n-pentyl

### Table 233

Compounds of the formula I.20 in which R¹ for each compound corresponds to one row of Table A and R² is n-pentyl

### Table 234

Compounds of the formula I.21 in which R¹ for each compound corresponds to one row of Table A and R² is n-pentyl

### Table 235

Compounds of the formula I.22 in which R¹ for each compound corresponds to one row of Table A and R² is n-pentyl

### Table 236

Compounds of the formula I.23 in which R¹ for each compound corresponds to one row of Table A and R² is n-pentyl

### Table 237

Compounds of the formula I.24 in which R¹ for each compound corresponds to one row of Table A and R² is n-pentyl

### Table 238

Compounds of the formula I.25 in which R¹ for each compound corresponds to one row of Table A and R² is n-pentyl

### Table 239

Compounds of the formula I.26 in which R¹ for each compound corresponds to one row of Table A and R² is n-pentyl

### Table 240

Compounds of the formula I.27 in which R¹ for each compound corresponds to one row of Table A and R² is n-pentyl

### Table 241

Compounds of the formula I.28 in which R¹ for each compound corresponds to one row of Table A and R² is n-pentyl

### Table 242

Compounds of the formula I.29 in which R¹ for each compound corresponds to one row of Table A and R² is n-pentyl

### Table 243

Compounds of the formula I.30 in which R¹ for each compound corresponds to one row of Table A and R² is n-pentyl

### Table 244

Compounds of the formula I.31 in which R¹ for each compound corresponds to one row of Table A and R² is n-pentyl

### Table 245

Compounds of the formula I.32 in which R¹ for each compound corresponds to one row of Table A and R² is n-pentyl

### Table 246

Compounds of the formula I.33 in which R¹ for each compound corresponds to one row of Table A and R² is n-pentyl

### Table 247

Compounds of the formula I.34 in which R¹ for each compound corresponds to one row of Table A, R² is n-pentyl and R is methyl

### Table 248

Compounds of the formula I.35 in which R¹ for each compound corresponds to one row of Table A, R² is n-pentyl and R, R^{A} and R^{A′} are methyl

### Table 249

Compounds of the formula I.36 in which R¹ for each compound corresponds to one row of Table A and R² is n-pentyl

### Table 250

Compounds of the formula I.37 in which R¹ for each compound corresponds to one row of Table A and R² is n-pentyl

### Table 251

Compounds of the formula I.38 in which R¹ for each compound corresponds to one row of Table A, R² is n-pentyl and R is hydrogen

### Table 252

Compounds of the formula I.38 in which R¹ for each compound corresponds to one row of Table A, R² is n-pentyl and R is methyl

### Table 253

Compounds of the formula I.1 in which R¹ for each compound corresponds to one row of Table A, D is methyl and R² is n-hexyl

### Table 254

Compounds of the formula I.2 in which R¹ for each compound corresponds to one row of Table A, D is methyl and R² is n-hexyl

### Table 255

Compounds of the formula I.3 in which R¹ for each compound corresponds to one row of Table A and R² is n-hexyl

### Table 256

Compounds of the formula I.4 in which R¹ for each compound corresponds to one row of Table A and R² is n-hexyl

### Table 257

Compounds of the formula I.5 in which R¹ for each compound corresponds to one row of Table A and R² is n-hexyl

### Table 258

Compounds of the formula I.6 in which R¹ for each compound corresponds to one row of Table A and R² is n-hexyl

### Table 259

Compounds of the formula I.7 in which R¹ for each compounds corresponds to one row of Table A and R² is n-hexyl

### Table 260

Compounds of the formula I.8 in which R¹ for each compound corresponds to one row of Table A and R² is n-hexyl

### Table 261

Compounds of the formula I.9 in which R¹ for each compound corresponds to one row of Table A and R² is n-hexyl

### Table 262

Compounds of the formula I.10 in which R¹ for each compound corresponds to one row of Table A and R² is n-hexyl

### Table 263

Compounds of the formula I.11 in which R¹ for each compound corresponds to one row of Table A and R² is n-hexyl

### Table 264

Compounds of the formula I.12 in which R¹ for each compound corresponds to one row of Table A and R² is n-hexyl

### Table 265

Compounds of the formula I.13 in which R¹ for each compound corresponds to one row of Table A, R² is n-hexyl and R is methyl

### Table 266

Compounds of the formula I.13 in which R¹ for each compound corresponds to one row of Table A, R² is n-hexyl and R is ethyl

### Table 267

Compounds of the formula I.13 in which R¹ for each compound corresponds to one row of Table A, R² is n-hexyl and R is n-propyl

### Table 268

Compounds of the formula I.13 in which R¹ for each compound corresponds to one row of Table A, R² is n-hexyl and R is isopropyl

### Table 269

Compounds of the formula I.14 in which R¹ for each compound corresponds to one row of Table A and R² is n-hexyl

### Table 270

Compounds of the formula I.15 in which R¹ for each compound corresponds to one row of Table A and R² is n-hexyl

### Table 271

Compounds of the formula I.16 in which R¹ for each compound corresponds to one row of Table A and R² is n-hexyl

### Table 272

Compounds of the formula I.17 in which R¹ for each compound corresponds to one row of Table A and R² is n-hexyl

### Table 273

Compounds of the formula I.18 in which R¹ for each compound corresponds to one row of Table A and R² is n-hexyl

### Table 274

Compounds of the formula I.19 in which R¹ for each compound corresponds to one row of Table A and R² is n-hexyl

### Table 275

Compounds of the formula I.20 in which R¹ for each compound corresponds to one row of Table A and R² is n-hexyl

### Table 276

Compounds of the formula I.21 in which R¹ for each compound corresponds to one row of Table A and R² is n-hexyl

### Table 277

Compounds of the formula I.22 in which R¹ for each compound corresponds to one row of Table A and R² is n-hexyl

### Table 278

Compounds of the formula I.23 in which R¹ for each compound corresponds to one row of Table A and R² is n-hexyl

### Table 279

Compounds of the formula I.24 in which R¹ for each compound corresponds to one row of Table A and R² is n-hexyl

### Table 280

Compounds of the formula I.25 in which R¹ for each compound corresponds to one row of Table A and R² is n-hexyl

### Table 281

Compounds of the formula I.26 in which R¹ for each compound corresponds to one row of Table A and R² is n-hexyl

### Table 282

Compounds of the formula I.27 in which R¹ for each compound corresponds to one row of Table A and R² is n-hexyl

### Table 283

Compounds of the formula I.28 in which R¹ for each compound corresponds to one row of Table A and R² is n-hexyl

### Table 284

Compounds of the formula I.29 in which R¹ for each compound corresponds to one row of Table A and R² is n-hexyl

### Table 285

Compounds of the formula I.30 in which R¹ for each compound corresponds to one row of Table A and R² is n-hexyl

### Table 286

Compounds of the formula I.31 in which R¹ for each compound corresponds to one row of Table A and R² is n-hexyl

### Table 287

Compounds of the formula I.32 in which R¹ for each compound corresponds to one row of Table A and R² is n-hexyl.

### Table 288

Compounds of the formula I.33 in which R¹ for each compound corresponds to one row of Table A and R² is n-hexyl

### Table 289

Compounds of the formula I.34 in which R¹ for each compound corresponds to one row of Table A, R² is n-hexyl and R is methyl

### Table 290

Compounds of the formula I.35 in which R¹ for each compound corresponds to one row of Table A, R² is n-hexyl and R, R^{A} and R^{A′} are methyl

### Table 291

Compounds of the formula I.36 in which R¹ for each compound corresponds to one row of Table A and R² is n-hexyl

### Table 292

Compounds of the formula I.37 in which R¹ for each compound corresponds to one row of Table A and R² is n-hexyl

### Table 293

Compounds of the formula I.38 in which R¹ for each compound corresponds to one row of Table A, R² is n-hexyl and R is hydrogen

### Table 294

Compounds of the formula I.38 in which R¹ for each compound corresponds to one row of Table A, R² is n-hexyl and R is methyl

### Table 295

Compounds of the formula I.1 in which R¹ for each compound corresponds to one row of Table A, D is methyl and R² is n-heptyl

### Table 296

Compounds of the formula I.2 in which R¹ for each compound corresponds to one row of Table A, D is methyl and R² is n-heptyl

### Table 297

Compounds of the formula I.3 in which R¹ for each compound corresponds to one row of Table A and R² is n-heptyl

### Table 298

Compounds of the formula I.4 in which R¹ for each compound corresponds to one row of Table A and R² is n-heptyl

### Table 299

Compounds of the formula I.5 in which R¹ for each compound corresponds to one row of Table A and R² is n-heptyl

### Table 300

Compounds of the formula I.6 in which R¹ for each compound corresponds to one row of Table A and R² is n-heptyl

### Table 301

Compounds of the formula I.7 in which R¹ for each compound corresponds to one row of Table A and R² is n-heptyl

### Table 302

Compounds of the formula I.8 in which R¹ for each compound corresponds to one row of Table A and R² is n-heptyl

### Table 303

Compounds of the formula I.9 in which R¹ for each compound corresponds to one row of Table A and R² is n-heptyl

### Table 304

Compounds of the formula I.10 in which R¹ for each compound corresponds to one row of Table A and R² is n-heptyl

### Table 305

Compounds of the formula I.11 in which R¹ for each compound corresponds to one row of Table A and R² is n-heptyl

### Table 306

Compounds of the formula I.12 in which R¹ for each compound corresponds to one row of Table A and R² is n-heptyl

### Table 307

Compounds of the formula I.13 in which R¹ for each compound corresponds to one row of Table A, R² is n-heptyl and R is methyl

### Table 308

Compounds of the formula I.13 in which R¹ for each compound corresponds to one row of Table A, R² is n-heptyl and R is ethyl

### Table 309

Compounds of the formula I.13 in which R¹ for each compound corresponds to one row of Table A, R² is n-heptyl and R is n-propyl

### Table 310

Compounds of the formula I.13 in which R¹ for each compound corresponds to one row of Table A, R² is n-heptyl and R is isopropyl

### Table 311

Compounds of the formula I.14 in which R¹ for each compound corresponds to one row of Table A and R² is n-heptyl

### Table 312

Compounds of the formula I.15 in which R¹ for each compound corresponds to one row of Table A and R² is n-heptyl

### Table 313

Compounds of the formula I.16 in which R¹ for each compound corresponds to one row of Table A and R² is n-heptyl

### Table 314

Compounds of the formula I.17 in which R¹ for each compound corresponds to one row of Table A and R² is n-heptyl

### Table 315

Compounds of the formula I.18 in which R¹ for each compound corresponds to one row of Table A and R² is n-heptyl

### Table 316

Compounds of the formula I.19 in which R¹ for each compound corresponds to one row of Table A and R² is n-heptyl

### Table 317

Compounds of the formula I.20 in which R¹ for each compound corresponds to one row of Table A and R² is n-heptyl

### Table 318

Compounds of the formula I.21 in which R¹ for each compound corresponds to one row of Table A and R² is n-heptyl

### Table 319

Compounds of the formula I.22 in which R¹ for each compound corresponds to one row of Table A and R² is n-heptyl

### Table 320

Compounds of the formula I.23 in which R¹ for each compound corresponds to one row of Table A and R² is n-heptyl

### Table 321

Compounds of the formula I.24 in which R¹ for each compound corresponds to one row of Table A and R² is n-heptyl.

### Table 322

Compounds of the formula I.25 in which R¹ for each compound corresponds to one row of Table A and R² is n-heptyl

### Table 323

Compounds of the formula I.26 in which R¹ for each compound corresponds to one row of Table A and R² is n-heptyl

### Table 324

Compounds of the formula I.27 in which R¹ for each compound corresponds to one row of Table A and R² is n-heptyl

### Table 325

Compounds of the formula I.28 in which R¹ for each compound corresponds to one row of Table A and R² is n-heptyl

### Table 326

Compounds of the formula I.29 in which R¹ for each compound corresponds to one row of Table A and R² is n-heptyl

### Table 327

Compounds of the formula I.30 in which R¹ for each compound corresponds to one row of Table A and R² is n-heptyl

### Table 328

Compounds of the formula I.31 in which R¹ for each compound corresponds to one row of Table A and R² is n-heptyl

### Table 329

Compounds of the formula I.32 in which R¹ for each compound corresponds to one row of Table A and R² is n-heptyl

### Table 330

Compounds of the formula I.33 in which R¹ for each compound corresponds to one row of Table A and R² is n-heptyl

### Table 331

Compounds of the formula I.34 in which R¹ for each compound corresponds to one row of Table A, R² is n-heptyl and R is methyl

### Table 332

Compounds of the formula I.35 in which R¹ for each compound corresponds to one row of Table A, R² is n-heptyl and R, R^{A} and R^{A′} are methyl

### Table 333

Compounds of the formula I.36 in which R¹ for each compound corresponds to one row of Table A and R² is n-heptyl

### Table 334

Compounds of the formula I.37 in which R¹ for each compound corresponds to one row of Table A and R² is n-heptyl

### Table 335

Compounds of the formula I.38 in which R¹ for each compound corresponds to one row of Table A, R² is n-heptyl and R is hydrogen

### Table 336

Compounds of the formula I.38 in which R¹ for each compound corresponds to one row of Table A, R² is n-heptyl and R is methyl

### Table 337

Compounds of the formula I.1 in which R¹ for each compound corresponds to one row of Table A, D is methyl and R² is n-octyl

### Table 338

Compounds of the formula I.2 in which R¹ for each compound corresponds to one row of Table A, D is methyl and R² is n-octyl

### Table 339

Compounds of the formula I.3 in which R¹ for each compound corresponds to one row of Table A and R² is n-octyl

### Table 340

Compounds of the formula I.4 in which R¹ for each compound corresponds to one row of Table A and R² is n-octyl

### Table 341

Compounds of the formula I.5 in which R¹ for each compound corresponds to one row of Table A and R² is n-octyl

### Table 342

Compounds of the formula I.6 in which R¹ for each compound corresponds to one row of Table A and R² is n-octyl

### Table 343

Compounds of the formula I.7 in which R¹ for each compound corresponds to one row of Table A and R² is n-octyl

### Table 344

Compounds of the formula I.8 in which R¹ for each compound corresponds to one row of Table A and R² is n-octyl

### Table 345

Compounds of the formula I.9 in which R¹ for each compound corresponds to one row of Table A and R² is n-octyl

### Table 346

Compounds of the formula I.10 in which R¹ for each compound corresponds to one row of Table A and R² is n-octyl

### Table 347

Compounds of the formula I.11 in which R¹ for each compound corresponds to one row of Table A and R² is n-octyl

### Table 348

Compounds of the formula I.12 in which R¹ for each compound corresponds to one row of Table A and R² is n-octyl

### Table 349

Compounds of the formula I.13 in which R¹ for each compound corresponds to one row of Table A, R² is n-octyl and R is methyl

### Table 350

Compounds of the formula I.13 in which R¹ for each compound corresponds to one row of Table A, R² is n-octyl and R is ethyl

### Table 351

Compounds of the formula I.13 in which R¹ for each compound corresponds to one row of Table A, R² is n-octyl and R is n-propyl

### Table 352

Compounds of the formula I.13 in which R¹ for each compound corresponds to one row of Table A, R² is n-octyl and R is isopropyl

### Table 353

Compounds of the formula I.14 in which R¹ for each compound corresponds to one row of Table A and R² is n-octyl

### Table 354

Compounds of the formula I.15 in which R¹ for each compound corresponds to one row of Table A and R² is n-octyl

### Table 355

Compounds of the formula I.16 in which R¹ for each compound corresponds to one row of Table A and R² is n-octyl

### Table 356

Compounds of the formula I.17 in which R¹ for each compound corresponds to one row of Table A and R² is n-octyl

### Table 357

Compounds of the formula I.18 in which R¹ for each compound corresponds to one row of Table A and R² is n-octyl

### Table 358

Compounds of the formula I.19 in which R¹ for each compound corresponds to one row of Table A and R² is n-octyl

### Table 359

Compounds of the formula I.20 in which R¹ for each compound corresponds to one row of Table A and R² is n-octyl

### Table 360

Compounds of the formula I.21 in which R¹ for each compound corresponds to one row of Table A and R² is n-octyl

### Table 361

Compounds of the formula I.22 in which R¹ for each compound corresponds to one row of Table A and R² is n-octyl

### Table 362

Compounds of the formula I.23 in which R¹ for each compound corresponds to one row of Table A and R² is n-octyl

### Table 363

Compounds of the formula I.24 in which R¹ for each compound corresponds to one row of Table A and R² is n-octyl

### Table 364

Compounds of the formula I.25 in which R¹ for each compound corresponds to one row of Table A and R² is n-octyl

### Table 365

Compounds of the formula I.26 in which R¹ for each compound corresponds to one row of Table A and R² is n-octyl

### Table 366

Compounds of the formula I.27 in which R¹ for each compound corresponds to one row of Table A and R² is n-octyl

### Table 367

Compounds of the formula I.28 in which R¹ for each compound corresponds to one row of Table A and R² is n-octyl

### Table 368

Compounds of the formula I.29 in which R¹ for each compound corresponds to one row of Table A and R² is n-octyl

### Table 369

Compounds of the formula I.30 in which R¹ for each compound corresponds to one row of Table A and R² is n-octyl

### Table 370

Compounds of the formula I.31 in which R¹ for each compound corresponds to one row of Table A and R² is n-octyl

### Table 371

Compounds of the formula I.32 in which R¹ for each compound corresponds to one row of Table A and R² is n-octyl

### Table 372

Compounds of the formula I.33 in which R¹ for each compound corresponds to one row of Table A and R² is n-octyl

### Table 373

Compounds of the formula I.34 in which R¹ for each compound corresponds to one row of Table A, R² is n-octyl and R is methyl

### Table 374

Compounds of the formula I.35 in which R¹ for each compound corresponds to one row of Table A, R² is n-octyl and R, R^{A} and R^{A′} are methyl

### Table 375

Compounds of the formula I.36 in which R¹ for each compound corresponds to one row of Table A and R² is n-octyl

### Table 376

Compounds of the formula I.37 in which R¹ for each compound corresponds to one row of Table A and R² is n-octyl

### Table 377

Compounds of the formula I.38 in which R¹ for each compound corresponds to one row of Table A, R² is n-octyl and R is hydrogen

### Table 378

Compounds of the formula I.38 in which R¹ for each compound corresponds to one row of Table A, R² is n-octyl and R is methyl

### Table 379

Compounds of the formula I.1 in which R¹ for each compound corresponds to one row of Table A, D is methyl and R² is n-nonyl

### Table 380

Compounds of the formula I.2 in which R¹ for each compound corresponds to one row of Table A, D is methyl and R² is n-nonyl

### Table 381

Compounds of the formula I.3 in which R¹ for each compound corresponds to one row of Table A and R² is n-nonyl

### Table 382

Compounds of the formula I.4 in which R¹ for each compound corresponds to one row of Table A and R² is n-nonyl

### Table 383

Compounds of the formula I.5 in which R¹ for each compound corresponds to one row of Table A and R² is n-nonyl

### Table 384

Compounds of the formula I.6 in which R¹ for each compound corresponds to one row of Table A and R² is n-nonyl

### Table 385

Compounds of the formula I.7 in which R¹ for each compound corresponds to one row of Table A and R² is n-nonyl

### Table 386

Compounds of the formula I.8 in which R¹ for each compound corresponds to one row of Table A and R² is n-nonyl

### Table 387

Compounds of the formula I.9 in which R¹ for each compound corresponds to one row of Table A and R² is n-nonyl

### Table 388

Compounds of the formula I.10 in which R¹ for each compound corresponds to one row of Table A and R² is n-nonyl

### Table 389

Compounds of the formula I.11 in which R¹ for each compound corresponds to one row of Table A and R² is n-nonyl

### Table 390

Compounds of the formula I.12 in which R¹ for each compound corresponds to one row of Table A and R² is n-nonyl

### Table 391

Compounds of the formula I.13 in which R¹ for each compound corresponds to one row of Table A, R² is n-nonyl and R is methyl

### Table 392

Compounds of the formula I.13 in which R¹ for each compound corresponds to one row of Table A, R² is n-nonyl and R is ethyl

### Table 393

Compounds of the formula I.13 in which R¹ for each compound corresponds to one row of Table A, R² is n-nonyl and R is n-propyl

### Table 394

Compounds of the formula I.13 in which R¹ for each compound corresponds to one row of Table A, R² is n-nonyl and R is isopropyl

### Table 395

Compounds of the formula I.14 in which R¹ for each compound corresponds to one row of Table A and R² is n-nonyl

### Table 396

Compounds of the formula I.15 in which R¹ for each compound corresponds to one row of Table A and R² is n-nonyl

### Table 397

Compounds of the formula I.16 in which R¹ for each compound corresponds to one row of Table A and R² is n-nonyl

### Table 398

Compounds of the formula I.17 in which R¹ for each compound corresponds to one row of Table A and R² is n-nonyl

### Table 399

Compounds of the formula I.18 in which R¹ for each compound corresponds to one row of Table A and R² is n-nonyl

### Table 400

Compounds of the formula I.19 in which R¹ for each compound corresponds to one row of Table A and R² is n-nonyl

### Table 401

Compounds of the formula I.20 in which R¹ for each compound corresponds to one row of Table A and R² is n-nonyl

### Table 402

Compounds of the formula I.21 in which R¹ for each compound corresponds to one row of Table A and R² is n-nonyl

### Table 403

Compounds of the formula I.22 in which R¹ for each compound corresponds to one row of Table A and R² n-nonyl

### Table 404

Compounds of the formula I.23 in which R¹ for each compound corresponds to one row of Table A and R² is n-nonyl

### Table 405

Compounds of the formula I.24 in which R¹ for each compound corresponds to one row of Table A and R² is n-nonyl

### Table 406

Compounds of the formula I.25 in which R¹ for each compound corresponds to one row of Table A and R² is n-nonyl

### Table 407

Compounds of the formula I.26 in which R¹ for each compound corresponds to one row of Table A and R² is n-nonyl

### Table 408

Compounds of the formula I.27 in which R¹ for each compound corresponds to one row of Table A and R² is n-nonyl

### Table 409

Compounds of the formula I.28 in which R¹ for each compound corresponds to one row of Table A and R² is n-nonyl

### Table 410

Compounds of the formula I.29 in which R¹ for each compound corresponds to one row of Table A and R² is n-nonyl

### Table 411

Compounds of the formula I.30 in which R¹ for each compound corresponds to one row of Table A and R² is n-nonyl

### Table 412

Compounds of the formula I.31 in which R¹ for each compound corresponds to one row of Table A and R² is n-nonyl

### Table 413

Compounds of the formula I.32 in which R¹ for each compound corresponds to one row of Table A and R² is n-nonyl

### Table 414

Compounds of the formula I.33 in which R¹ for each compound corresponds to one row of Table A and R² is n-nonyl

### Table 415

Compounds of the formula I.34 in which R¹ for each compound corresponds to one row of Table A, R² is n-nonyl and R is methyl

### Table 416

Compounds of the formula I.35 in which R¹ for each compound corresponds to one row of Table A, R² is n-nonyl and R, R^{A} and R^{A′} are methyl

### Table 417

Compounds of the formula I.36 in which R¹ for each compound corresponds to one row of Table A and R² is n-nonyl

### Table 418

Compounds of the formula I.37 in which R¹ for each compound corresponds to one row of Table A and R² is n-nonyl

### Table 419

Compounds of the formula I.38 in which R¹ for each compound corresponds to one row of Table A, R² is n-nonyl and R is hydrogen

### Table 420

Compounds of the formula I.38 in which R¹ for each compound corresponds to one row of Table A, R² is n-nonyl and R is methyl

### Table 421

Compounds of the formula I.1 in which R¹ for each compound corresponds to one row of Table A, D is methyl and R² is n-decyl

### Table 422

Compounds of the formula I.2 in which R¹ for each compound corresponds to one row of Table A, D is methyl and R² is n-decyl

### Table 423

Compounds of the formula I.3 in which R¹ for each compound corresponds to one row of Table A and R² is n-decyl

### Table 424

Compounds of the formula I.4 in which R¹ for each compound corresponds to one row of Table A and R² is n-decyl

### Table 425

Compounds of the formula I.5 in which R¹ for each compound corresponds to one row of Table A and R² is n-decyl

### Table 426

Compounds of the formula I.6 in which R¹ for each compound corresponds to one row of Table A and R² is n-decyl

### Table 427

Compounds of the formula I.7 in which R¹ for each compound corresponds to one row of Table A and R² is n-decyl

### Table 428

Compounds of the formula I.8 in which R¹ for each compound corresponds to one row of Table A and R² is n-decyl

### Table 429

Compounds of the formula I.9 in which R¹ for each compound corresponds to one row of Table A and R² is n-decyl

### Table 430

Compounds of the formula I.10 in which R¹ for each compound corresponds to one row of Table A and R² is n-decyl

### Table 431

Compounds of the formula I.11 in which R¹ for each compound corresponds to one row of Table A and R² is n-decyl

### Table 432

Compounds of the formula I.12 in which R¹ for each compound corresponds to one row of Table A and R² is n-decyl

### Table 433

Compounds of the formula I.13 in which R¹ for each compound corresponds to one row of Table A, R² is n-decyl and R is methyl

### Table 434

Compounds of the formula I.13 in which R¹ for each compound corresponds to one row of Table A, R² is n-decyl and R is ethyl

### Table 435

Compounds of the formula I.13 in which R¹ for each compound corresponds to one row of Table A, R² is n-decyl and R is n-propyl

### Table 436

Compounds of the formula I.13 in which R¹ for each compound corresponds to one row of Table A, R² is n-decyl and R is isopropyl

### Table 437

Compounds of the formula I.14 in which R¹ for each compound corresponds to one row of Table A and R² is n-decyl

### Table 438

Compounds of the formula I.15 in which R¹ for each compound corresponds to one row of Table A and R² is n-decyl

### Table 439

Compounds of the formula I.16 in which R¹ for each compound corresponds to one row of Table A and R² is n-decyl

### Table 440

Compounds of the formula I.17 in which R¹ for each compound corresponds to one row of Table A and R² is n-decyl

### Table 441

Compounds of the formula I.18 in which R¹ for each compound corresponds to one row of Table A and R² is n-decyl

### Table 442

Compounds of the formula I.19 in which R¹ for each compound corresponds to one row of Table A and R² is n-decyl

### Table 443

Compounds of the formula I.20 in which R¹ for each compound corresponds to one row of Table A and R² is n-decyl

### Table 444

Compounds of the formula I.21 in which R¹ for each compound corresponds to one row of Table A and R² is n-decyl

### Table 445

Compounds of the formula I.22 in which R¹ for each compound corresponds to one row of Table A and R² is n-decyl

### Table 446

Compounds of the formula I.23 in which R¹ for each compound corresponds to one row of Table A and R² is n-decyl

### Table 447

Compounds of the formula I.24 in which R¹ for each compound corresponds to one row of Table A and R² is n-decyl

### Table 448

Compounds of the formula I.25 in which R¹ for each compound corresponds to one row of Table A and R² is n-decyl

### Table 449

Compounds of the formula I.26 in which R¹ for each compound corresponds to one row of Table A and R² is n-decyl

### Table 450

Compounds of the formula I.27 in which R¹ for each compound corresponds to one row of Table A and R² is n-decyl

### Table 451

Compounds of the formula I.28 in which R¹ for each compound corresponds to one row of Table A and R² is n-decyl

### Table 452

Compounds of the formula I.29 in which R¹ for each compound corresponds to one row of Table A and R² is n-decyl

### Table 453

Compounds of the formula I.30 in which R¹ for each compound corresponds to one row of Table A and R² is n-decyl

### Table 454

Compounds of the formula I.31 in which R¹ for each compound corresponds to one row of Table A and R² is n-decyl

### Table 455

Compounds of the formula I.32 in which R¹ for each compound corresponds to one row of Table A and R² is n-decyl

### Table 456

Compounds of the formula I.33 in which R¹ for each compound corresponds to one row of Table A and R² is n-decyl

### Table 457

Compounds of the formula I.34 in which R¹ for each compound corresponds to one row of Table A, R² is n-decyl and R is methyl

### Table 458

Compounds of the formula I.35 in which R¹ for each compound corresponds to one row of Table A, R² is n-decyl and R, R^{A} and R^{A′} are methyl

### Table 459

Compounds of the formula I.36 in which R¹ for each compound corresponds to one row of Table A and R² is n-decyl

### Table 460

Compounds of the formula I.37 in which R¹ for each compound corresponds to one row of Table A and R² is n-decyl

### Table 461

Compounds of the formula I.38 in which R¹ for each compound corresponds to one row of Table A, R² is n-decyl and R is hydrogen

### Table 462

Compounds of the formula I.38 in which R¹ for each compound corresponds to one row of Table A, R² is n-decyl and R is methyl

### Table 463

Compounds of the formula I.1 in which R¹ for each compound corresponds to one row of Table A, D is methyl and R² is methoxymethyl

### Table 464

Compounds of the formula I.2 in which R¹ for each compound corresponds to one row of Table A, D is methyl and R² is methoxymethyl

### Table 465

Compounds of the formula I.3 in which R¹ for each compound corresponds to one row of Table A and R² is methoxymethyl

### Table 466

Compounds of the formula I.4 in which R¹ for each compound corresponds to one row of Table A and R² is methoxymethyl

### Table 467

Compounds of the formula I.5 in which R¹ for each compound corresponds to one row of Table A and R² is methoxymethyl

### Table 468

Compounds of the formula I.6 in which R¹ for each compound corresponds to one row of Table A and R² is methoxymethyl

### Table 469

Compounds of the formula I.7 in which R¹ for each compound corresponds to one row of Table A and R² is methoxymethyl

### Table 470

Compounds of the formula I.8 in which R¹ for each compound corresponds to one row of Table A and R² is methoxymethyl

### Table 471

Compounds of the formula I.9 in which R¹ for each compound corresponds to one row of Table A and R² is methoxymethyl

### Table 472

Compounds of the formula I.10 in which R¹ for each compound corresponds to one row of Table A and R² is methoxymethyl

### Table 473

Compounds of the formula I.11 in which R¹ for each compound corresponds to one row of Table A and R² is methoxymethyl

### Table 474

Compounds of the formula I.12 in which R¹ for each compound corresponds to one row of Table A and R² is methoxymethyl

### Table 475

Compounds of the formula I.13 in which R¹ for each compound corresponds to one row of Table A, R² is methoxymethyl and R is methyl

### Table 476

Compounds of the formula I.13 in which R¹ for each compound corresponds to one row of Table A, R² is methoxymethyl and R is ethyl

### Table 477

Compounds of the formula I.13 in which R¹ for each compound corresponds to one row of Table A, R² is methoxymethyl and R is n-propyl

### Table 478

Compounds of the formula I.13 in which R¹ for each compound corresponds to one row of Table A, R² is methoxymethyl and R is isopropyl

### Table 479

Compounds of the formula I.14 in which R¹ for each compound corresponds to one row of Table A and R² is methoxymethyl

### Table 480

Compounds of the formula I.15 in which R¹ for each compound corresponds to one row of Table A and R² is methoxymethyl

### Table 481

Compounds of the formula I.16 in which R¹ for each compound corresponds to one row of Table A and R² is methoxymethyl

### Table 482

Compounds of the formula I.17 in which R¹ for each compound corresponds to one row of Table A and R² is methoxymethyl

### Table 483

Compounds of the formula I.18 in which R¹ for each compound corresponds to one row of Table A and R² is methoxymethyl

### Table 484

Compounds of the formula I.19 in which R¹ for each compound corresponds to one row of Table A and R² is methoxymethyl

### Table 485

Compounds of the formula I.20 in which R¹ for each compound corresponds to one row of Table A and R² is methoxymethyl

### Table 486

Compounds of the formula I.21 in which R¹ for each compound corresponds to one row of Table A and R² is methoxymethyl

### Table 487

Compounds of the formula I.22 in which R¹ for each compound corresponds to one row of Table A and R² is methoxymethyl

### Table 488

Compounds of the formula I.23 in which R¹ for each compound corresponds to one row of Table A and R² is methoxymethyl

### Table 489

Compounds of the formula I.24 in which R¹ for each compound corresponds to one row of Table A and R² is methoxymethyl

### Table 490

Compounds of the formula I.25 in which R¹ for each compound corresponds to one row of Table A and R² is methoxymethyl

### Table 491

Compounds of the formula I.26 in which R¹ for each compound corresponds to one row of Table A and R² is methoxymethyl

### Table 492

Compounds of the formula I.27 in which R¹ for each compound corresponds to one row of Table A and R² is methoxymethyl

### Table 493

Compounds of the formula I.28 in which R¹ for each compound corresponds to one row of Table A and R² is methoxymethyl

### Table 494

Compounds of the formula I.29 in which R¹ for each compound corresponds to one row of Table A and R² is methoxymethyl

### Table 495

Compounds of the formula I.30 in which R¹ for each compound corresponds to one row of Table A and R² is methoxymethyl

### Table 496

Compounds of the formula I.31 in which R¹ for each compound corresponds to one row of Table A and R² is methoxymethyl

### Table 497

Compounds of the formula I.32 in which R¹ for each compound corresponds to one row of Table A and R² is methoxymethyl

### Table 498

Compounds of the formula I.33 in which R¹ for each compound corresponds to one row of Table A and R² is methoxymethyl

### Table 499

Compounds of the formula I.34 in which R¹ for each compound corresponds to one row of Table A, R² is methoxymethyl and R is methyl

### Table 500

Compounds of the formula I.35 in which R¹ for each compound corresponds to one row of Table A, R² is methoxymethyl and R, R^{A} and R^{A′} are methyl

### Table 501

Compounds of the formula I.36 in which R¹ for each compound corresponds to one row of Table A and R² is methoxymethyl

### Table 502

Compounds of the formula I.37 in which R¹ for each compound corresponds to one row of Table A and R² is methoxymethyl

### Table 503

Compounds of the formula I.38 in which R¹ for each compound corresponds to one row of Table A, R² is methoxymethyl and R is hydrogen

### Table 504

Compounds of the formula I.38 in which R¹ for each compound corresponds to one row of Table A, R² is methoxymethyl and R is methyl

### Table 505

Compounds of the formula I.1 in which R¹ for each compound corresponds to one row of Table A, D is methyl and R² is ethoxymethyl

### Table 506

Compounds of the formula I.2 in which R¹ for each compound corresponds to one row of Table A, D is methyl and R² is ethoxymethyl

### Table 507

Compounds of the formula I.3 in which R¹ for each compound corresponds to one row of Table A and R² is ethoxymethyl

### Table 508

Compounds of the formula I.4 in which R¹ for each compound corresponds to one row of Table A and R² is ethoxymethyl

### Table 509

Compounds of the formula I.5 in which R¹ for each compound corresponds to one row of Table A and R² is ethoxymethyl

### Table 510

Compounds of the formula I.6 in which R¹ for each compound corresponds to one row of Table A and R² is ethoxymethyl

### Table 511

Compounds of the formula I.7 in which R¹ for each compound corresponds to one row of Table A and R² is ethoxymethyl

### Table 512

Compounds of the formula I.8 in which R¹ for each compound corresponds to one row of Table A and R² is ethoxymethyl

### Table 513

Compounds of the formula I.9 in which R¹ for each compound corresponds to one row of Table A and R² is ethoxymethyl

### Table 514

Compounds of the formula I.10 in which R¹ for each compound corresponds to one row of Table A and R² is ethoxymethyl

### Table 515

Compounds of the formula I.11 in which R¹ for each compound corresponds to one row of Table A and R² is ethoxymethyl

### Table 516

Compounds of the formula I.12 in which R¹ for each compound corresponds to one row of Table A and R² is ethoxymethyl

### Table 517

Compounds of the formula I.13 in which R¹ for each compound corresponds to one row of Table A, R² is ethoxymethyl and R is methyl

### Table 518

Compounds of the formula I.13 in which R¹ for each compound corresponds to one row of Table A, R² is ethoxymethyl and R is ethyl

### Table 519

Compounds of the formula I.13 in which R¹ for each compound corresponds to one row of Table A, R² is ethoxymethyl and R is n-propyl

### Table 520

Compounds of the formula I.13 in which R¹ for each compound corresponds to one row of Table A, R² is ethoxymethyl and R is isopropyl

### Table 521

Compounds of the formula I.14 in which R¹ for each compound corresponds to one row of Table A and R² is ethoxymethyl

### Table 522

Compounds of the formula I.15 in which R¹ for each compound corresponds to one row of Table A and R² is ethoxymethyl

### Table 523

Compounds of the formula I.16 in which R¹ for each compound corresponds to one row of Table A and R² is ethoxymethyl

### Table 524

Compounds of the formula I.17 in which R¹ for each compound corresponds to one row of Table A and R² is ethoxymethyl

### Table 525

Compounds of the formula I.18 in which R¹ for each compound corresponds to one row of Table A and R² is ethoxymethyl

### Table 526

Compounds of the formula I.19 in which R¹ for each compound corresponds to one row of Table A and R² is ethoxymethyl

### Table 527

Compounds of the formula I.20 in which R¹ for each compound corresponds to one row of Table A and R² is ethoxymethyl

### Table 528

Compounds of the formula I.21 in which R¹ for each compound corresponds to one row of Table A and R² is ethoxymethyl

### Table 529

Compounds of the formula I.22 in which R¹ for each compound corresponds to one row of Table A and R² is ethoxymethyl

### Table 530

Compounds of the formula I.23 in which R¹ for each compound corresponds to one row of Table A and R² is ethoxymethyl

### Table 531

Compounds of the formula I.24 in which R¹ for each compound corresponds to one row of Table A and R² is ethoxymethyl

### Table 532

Compounds of the formula I.25 in which R¹ for each compound corresponds to one row of Table A and R² is ethoxymethyl

### Table 533

Compounds of the formula I.26 in which R¹ for each compound corresponds to one row of Table A and R² is ethoxymethyl

### Table 534

Compounds of the formula I.27 in which R¹ for each compound corresponds to one row of Table A and R² is ethoxymethyl

### Table 535

Compounds of the formula I.28 in which R¹ for each compound corresponds to one row of Table A and R² is ethoxymethyl

### Table 536

Compounds of the formula I.29 in which R¹ for each compound corresponds to one row of Table A and R² is ethoxymethyl

### Table 537

Compounds of the formula I.30 in which R¹ for each compound corresponds to one row of Table A and R² is ethoxymethyl

### Table 538

Compounds of the formula I.31 in which R¹ for each compound corresponds to one row of Table A and R² is ethoxymethyl

### Table 539

Compounds of the formula I.32 in which R¹ for each compound corresponds to one row of Table A and R² is ethoxymethyl

### Table 540

Compounds of the formula I.33 in which R¹ for each compound corresponds to one row of Table A and R² is ethoxymethyl

### Table 541

Compounds of the formula I.34 in which R¹ for each compound corresponds to one row of Table A, R² is ethoxymethyl and R is methyl

### Table 542

Compounds of the formula I.35 in which R¹ for each compound corresponds to one row of Table A, R² is ethoxymethyl and R, R^{A} and R^{A′} are methyl

### Table 543

Compounds of the formula I.36 in which R¹ for each compound corresponds to one row of Table A and R² is ethoxymethyl

### Table 544

Compounds of the formula I.37 in which R¹ for each compound corresponds to one row of Table A and R² is ethoxymethyl

### Table 545

Compounds of the formula I.38 in which R¹ for each compound corresponds to one row of Table A, R² is ethoxymethyl and R is hydrogen

### Table 546

Compounds of the formula I.38 in which R¹ for each compound corresponds to one row of Table A, R² is ethoxymethyl and R is methyl

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH₂CH₂CH₃ |
| A-6 | CH(CH₃)CH₂CH₃ |
| A-7 | CH₂CH(CH₃)₂ |
| A-8 | C(CH₃)₃ |
| A-9 | CH₂CH₂CH₂CH₂CH₃ |
| A-10 | CH(CH₃)CH₂CH₂CH₃ |
| A-11 | CH₂CH(CH₃)CH₂CH₃ |
| A-12 | CH₂CH₂CH(CH₃)CH₃ |
| A-13 | CH₂CH₂CH(CH₃)₂ |
| A-14 | CH(CH₃)CH(CH₃)CH₃ |
| A-15 | CH(CH₃)CH(CH₃)₂ |
| A-16 | CH₂C(CH₃)₃ |
| A-17 | CH₂CH₂CH₂CH₂CH₂CH₃ |
| A-18 | CH(CH₃)CH₂CH₂CH₂CH₃ |
| A-19 | CH₂CH(CH₃)CH₂CH₂CH₃ |
| A-20 | CH₂CH₂CH(CH₃)CH₂CH₃ |
| A-21 | CH₂CH₂CH(CH₃)₂CH₂ |
| A-22 | CH₂CH₂CH₂CH(CH₃)₂ |
| A-23 | CH(CH₃)CH(CH₃)CH₂CH₃ |
| A-24 | CH(CH₃)CH₂CH(CH₃)₂ |
| A-25 | CH₂CH₂C(CH₃)₃ |
| A-26 | CH(CH₃)CH₂CH(CH₃)CH₃ |
| A-27 | CH₂CH₂CH₂CH₂CH₂CH₂CH₃ |
| A-28 | CH(CH₃)CH₂CH₂CH₂CH₂CH₃ |
| A-29 | CH₂CH(CH₃)CH₂CH₂CH₂CH₃ |
| A-30 | CH₂CH₂CH(CH₃)CH₂CH₂CH₃ |
| A-31 | CH₂CH₂CH₂CH(CH₃)CH₂CH₃ |
| A-32 | CH₂CH₂CH₂CH₂CH(CH₃)CH₃ |
| A-33 | CH₂CH₂CH₂CH₂CH(CH₃)₂ |
| A-34 | CH(CH₃)CH(CH₃)CH₂CH₂CH₃ |
| A-35 | CH₂CH(CH₃)CH(CH₃)CH₂CH₃ |
| A-36 | CH₂CH₂CH₂C(CH₃)₃ |
| A-37 | CH(CH₃)CH₂CH(CH₃)CH₂CH₃ |
| A-38 | CH₂CH(CH₃)CH(CH₃)CH₂CH₃ |
| A-39 | CH(CH₃)CH₂CH₂CH(CH₃)CH₃ |
| A-40 | CH₂CH₂CH₂CH₂CH₂CH₂CH₂CH₃ |
| A-41 | CH(CH₃)CH₂CH₂CH₂CH₂CH₂CH₃ |
| A-42 | CH₂CH(CH₃)CH₂CH₂CH₂CH₂CH₃ |
| A-43 | CH₂CH₂CH(CH₃)CH₂CH₂CH₂CH₃ |
| A-44 | CH₂CH₂CH₂CH(CH₃)CH₂CH₂CH₃ |
| A-45 | CH₂CH₂CH₂CH₂CH(CH₃)CH₂CH₃ |
| A-46 | CH₂CH₂CH₂CH₂CH₂CH(CH₃)₂ |
| A-47 | CH₂CH₂CH₂CH₂C(CH₃)₃ |
| A-48 | CH(CH₃)CH(CH₃)CH₂CH₂CH₂CH₃ |
| A-49 | CH₂CH(CH₃)CH(CH₃)CH₂CH₂CH₃ |
| A-50 | CH₂CH₂CH₂C(CH₃)₂CH₂CH₃ |
| A-51 | CH(CH₃)CH₂CH(CH₃)CH₂CH₂CH₃ |
| A-52 | CH₂CH(CH₃)CH(CH₃)CH₂CH₂CH₃ |
| A-53 | CH(CH₃)CH₂CH₂CH(CH₃)CH₂CH₃ |
| A-54 | CH(CH₃)CH₂CH₂CH₂CH(CH₃)₂ |
| A-55 | CH₂CH₂CH(CH₃)CH₂C(CH₃)₃ |
| A-56 | CH₂CH₂CH₂CH₂CH₂CH₂CH₂CH₂CH₃ |
| A-57 | CH(CH₃)CH₂CH₂CH₂CH₂CH₂CH₂CH₃ |
| A-58 | CH₂CH(CH₃)CH₂CH₂CH₂CH₂CH₂CH₃ |
| A-59 | CH₂CH₂CH(CH₃)CH₂CH₂CH₂CH₂CH₃ |
| A-60 | CH₂CH₂CH₂CH(CH₃)CH₂CH₂CH₂CH₃ |
| A-61 | CH₂CH₂CH₂CH₂CH(CH₃)CH₂CH₂CH₃ |
| A-62 | CH₂CH₂CH₂CH₂CH₂CH₂C(CH₃)₃ |
| A-63 | CH(CH₃)CH(CH₃)CH₂CH₂CH₂CH₂CH₃ |
| A-64 | CH₂CH(CH₃)CH(CH₃)CH₂CH₂CH₂CH₃ |
| A-65 | CH₂CH₂CH₂C(CH₃)₂CH₂CH₂CH₃ |
| A-66 | CH(CH₃)CH₂CH(CH₃)CH₂CH₂CH₂CH₃ |
| A-67 | CH₂CH(CH₃)CH(CH₃)CH₂CH₂CH₂CH₃ |
| A-68 | CH(CH₃)CH₂CH₂CH(CH₃)CH₂CH₂CH₃ |
| A-69 | CH(CH₃)CH₂CH₂CH₂C(CH₃)₃ |
| A-70 | CH₂CH(CH₃)CH₂CH₂CH(CH₃)₃ |
| A-71 | CH(CH₃)CH₂CH₂CH₂CH₂CH(CH₃)₂ |
| A-72 | CH₂CH(CH₃)CH₂CH₂CH₂CH(CH₃)₂ |
| A-73 | CH₂CH₂CH₂CH₂CH₂CH₂CH₂CH₂CH₂CH₃ |
| A-74 | CH(CH₃)CH₂CH₂CH₂CH₂CH₂CH₂CH₂CH₃ |
| A-75 | CH₂CH(CH₃)CH₂CH₂CH₂CH₂CH₂CH₂CH₃ |
| A-76 | CH₂CH₂CH(CH₃)CH₂CH₂CH₂CH₂CH₂CH₃ |
| A-77 | CH₂CH₂CH(CH₃)CH₂CH₂CH₂CH₂CH₂ |
| A-78 | CH₂CH₂CH₂CH(CH₃)CH₂CH₂CH₂CH₃ |
| A-79 | CH₂CH₂CH₂CH₂CH₂CH₂C(CH₃)₃ |
| A-80 | CH(CH₃)CH(CH₃)CH₂CH₂CH₂CH₂CH₂CH₃ |
| A-81 | CH₂CH(CH₃)CH(CH₃)CH₂CH₂CH₂CH₂CH₃ |
| A-82 | CH₂CH₂CH₂C(CH₃)₂CH₂CH₂CH₂CH₃ |
| A-83 | CH(CH₃)CH₂CH(CH₃)CH₂CH₂CH₂CH₂CH₃ |
| A-84 | CH₂CH(CH₃)CH(CH₃)CH₂CH₂CH₂CH₂CH₃ |
| A-85 | CH(CH₃)CH₂CH₂CH(CH₃)CH₂CH₂CH₂CH₃ |
| A-86 | CH(CH₃)CH₂CH₂CH₂CH(CH₃)CH₂CH₂CH₃ |
| A-87 | CH(CH₃)CH₂CH₂CH₂CH₂CH(CH₃)CH₂CH₃ |
| A-88 | CH(CH₃)CH₂CH₂CH₂CH₂CH₂CH(CH₃)₂ |
| A-89 | CH(CH₃)CH₂CH₂CH₂CH₂CH₂C(CH₃)CH₃ |
| A-90 | CH₂CH(CH₃)CH₂CH₂CH₂CH₂CH(CH₃)CH₃ |
| A-91 | CH(CH₃)CH₂CH₂CH₂CH₂C(CH₃)₃ |
| A-92 | CH₂CH(CH₃)CH₂CH₂CH₂C(CH₃)₃ |
| A-93 | CH₂CH₂CH₂CH₂CH₂CH₂CH₂CH₂CH₂CH₂CH₃ |
| A-94 | CH(CH₃)CH₂CH₂CH₂CH₂CH₂CH₂CH₂CH₂CH₃ |
| A-95 | CH₂CH(CH₃)CH₂CH₂CH₂CH₂CH₂CH₂CH₂CH₃ |
| A-96 | CH₂CH₂CH(CH₃)CH₂CH₂CH₂CH₂CH₂CH₂CH₃ |
| A-97 | CH₂CH₂CH₂CH(CH₃)CH₂CH₂CH₂CH₂CH₂CH₃ |
| A-98 | CH₂CH₂CH₂CH₂CH(CH₃)CH₂CH₂CH₂CH₂CH₃ |
| A-99 | CH₂CH₂CH₂CH₂CH₂CH₂CH₂C(CH₃)₃ |
| A-100 | CH(CH₃)CH(CH₃)CH₂CH₂CH₂CH₂CH₂CH₂CH₃ |
| A-101 | CH₂CH(CH₃)CH(CH₃)CH₂CH₂CH₂CH₂CH₂CH₃ |
| A-102 | CH₂CH₂CH₂C(CH₃)₂CH₂CH₂CH₂CH₂CH₃ |
| A-103 | CH(CH₃)CH₂CH(CH₃)CH₂CH₂CH₂CH₂CH₂CH₃ |
| A-104 | CH₂CH(CH₃)CH(CH₃)CH₂CH₂CH₂CH₂CH₂CH₃ |
| A-105 | CH(CH₃)CH₂CH₂CH(CH₃)CH₂CH₂CH₂CH₂CH₃ |
| A-106 | CH(CH₃)CH₂CH₂CH₂CH(CH₃)CH₂CH₂CH₂CH₃ |
| A-107 | CH(CH₃)CH₂CH₂CH₂CH₂CH(CH₃)CH₂CH₂CH₃ |
| A-108 | CH(CH₃)CH₂CH₂CH₂CH₂CH₂CH(CH₃)CH₂CH₃ |
| A-109 | CH(CH₃)CH₂CH₂CH₂CH₂CH₂CH₂CH(CH₃)₂ |
| A-110 | CH₂CH(CH₃)CH₂CH₂CH₂CH₂CH(CH₃)CH₂CH₃ |
| A-111 | CH₂CH(CH₃)CH₂CH₂CH₂CH₂CH(CH₃)CH₂CH₃ |
| A-112 | CH₂CH₂CH(CH₃)CH₂CH₂CH₂CH₂CH(CH₃)₂ |
| A-113 | CH₂CH(CH₃)CH₂CH₂CH₂CH₂C(CH₃)₃ |
| A-114 | CH₂CH₂CH₂CH₂CH₂CH₂CH₂CH₂CH₂CH₂CH₂CH₃ |
| A-115 | CH(CH₃)CH₂CH₂CH₂CH₂CH₂CH₂CH₂CH₂CH₂CH₃ |
| A-116 | CH₂CH(CH₃)CH₂CH₂CH₂CH₂CH₂CH₂CH₂CH₂CH₃ |
| A-117 | CH₂CH₂CH(CH₃)CH₂CH₂CH₂CH₂CH₂CH₂CH₂CH₃ |
| A-118 | CH₂CH₂CH₂CH(CH₃)CH₂CH₂CH₂CH₂CH₂CH₂CH₂ |
| A-119 | CH₂CH₂CH₂CH₂CH(CH₃)CH₂CH₂CH₂CH₂CH₂CH₃ |
| A-120 | CH₂CH₂CH₂CH₂CH₂CH(CH₃)CH₂CH₂CH₂CH₂CH₃ |
| A-121 | CH₂CH₂CH₂CH₂CH₂CH₂CH(CH₃)CH₂CH₂CH₂CH₃ |
| A-122 | CH₂CH₂CH₂CH₂CH₂CH₂CH₂CH(CH₃)CH₂CH₂CH₃ |
| A-123 | CH₂CH₂CH₂CH₂CH₂CH₂CH₂CH₂CH(CH₃)CH₂CH₃ |
| A-124 | CH₂CH₂CH₂CH₂CH₂CH₂CH₂CH₂CH₂CH(CH₃)₂ |
| A-125 | CH(CH₃)CH(CH₃)CH₂CH₂CH₂CH₂CH₂CH₂CH₂CH₃ |
| A-126 | CH₂CH(CH₃)CH(CH₃)CH₂CH₂CH₂CH₂CH₂CH₂CH₃ |
| A-127 | CH₂CH₂CH₂C(CH₃)₂CH₂CH₂CH₂CH₂CH₂CH₃ |
| A-128 | CH₂CH₂CH₂CH₂CH(CH₃)CH₂CH₂CH₂CH₂CH₂CH₃ |
| A-129 | CH(CH₃)CH₂CH(CH₃)CH₂CH₂CH₂CH₂CH₂CH₂CH₃ |
| A-130 | CH(CH₃)CH₂CH₂CH(CH₃)CH₂CH₂CH₂CH₂CH₂CH₃ |
| A-131 | CH(CH₃)CH₂CH₂CH₂CH(CH₃)CH₂CH₂CH₂CH₂CH₃ |
| A-132 | CH(CH₃)CH₂CH₂CH₂CH₂CH(CH₃)CH₂CH₂CH₂CH₃ |
| A-133 | CH(CH₃)CH₂CH₂CH₂CH₂CH₂CH(CH₃)CH₂CH₂CH₃ |
| A-134 | CH(CH₃)CH₂CH₂CH₂CH₂CH₂CH₂CH(CH₃)CH₂CH₃ |
| A-135 | CH₂CH(CH₃)CH₂CH₂CH₂CH₂CH(CH₃)CH₂CH₂CH₃ |
| A-136 | CH₂CH₂CH(CH₃)CH₂CH₂CH₂CH₂CH(CH₃)CH₂CH₃ |
| A-137 | CH₂CH₂CH₂CH(CH₃)CH₂CH₂CH₂CH(CH₃)CH₂CH₃ |
| A-138 | CH₂CH(CH₃)CH₂CH₂CH₂CH₂CH₂C(CH₃)₃ |
| A-139 | CH₂CH₂CH₂—O—CH₃ |
| A-140 | CH₂CH₂CH₂—O—CH₂CH₃ |
| A-141 | CH₂CH₂CH₂—O—CH₂CH₂CH₃ |
| A-142 | CH₂CH₂CH₂—O—CH₂CH₂CH₂CH₃ |
| A-143 | CH₂CH₂CH₂—O—CH₂CH₂CH₂CH₂CH₃ |
| A-144 | CH₂CH₂CH₂—O—CH₂CH₂CH₂CH₂CH₂CH₃ |
| A-145 | CH₂CH₂CH₂—O—CH₂CH₂CH₂CH₂CH₂CH₂CH₃ |
| A-146 | CH₂CH₂CH₂—O—CH₂CH₂CH₂CH₂CH₂CH₂CH₂CH₃ |
| A-147 | CH₂CH₂CH₂—O—CH₂CH₂CH₂CH₂CH₂CH₂CH₂CH₂CH₃ |
| A-148 | CH₂CH₂CH₂—O—CH(CH₃)₂ |
| A-149 | CH₂CH₂CH₂—O—C(CH₃)₃ |
| A-150 | CH₂CH₂CH₂—O—CH₂C(CH₃)₃ |
| A-151 | CH₂CH₂CH₂—O—CH(CH₃)CH₂C(CH₃)₃ |
| A-152 | CH₂CH₂CH₂—O—CH(CH₂CH₃)CH₂C(CH₃)₃ |
| A-153 | CH₂CH₂CH₂—O—CH₂CH(CH₃)CH₂CH(CH₃)₂ |
| A-154 | CH₂CH₂CH₂—O—CH₂CH(CH₂CH₃)CH₂CH₂CH₃ |
| A-155 | CH₂CH₂CH₂—O—CH₂CH₂CH(CH₃)CH₂CH(CH₃)₂ |
| A-156 | CH₂CH₂CH₂—O—CH₂CH₂CH(CH₃)CH₂C(CH₃)₃ |
| A-157 | CH₂CH₂CH₂—O—CH₂CH₂CH(CH₃)CH₂CH₂CH(CH₃)₂ |
| A-158 | CH₂CH₂CH₂—O—CH₂CH₂CH(CH₃)CH₂CH₂CH₂CH(CH₃)₂ |
| A-159 | CH₂CH₂CH₂CH₂—O—CH₃ |
| A-160 | CH₂CH₂CH₂CH₂—O—CH₂CH₃ |
| A-161 | CH₂CH₂CH₂CH₂—O—CH₂CH₂CH₃ |
| A-162 | CH₂CH₂CH₂CH₂—O—CH₂CH₂CH₂CH₃ |
| A-163 | CH₂CH₂CH₂CH₂—O—CH₂CH₂CH₂CH₂CH₃ |
| A-164 | CH₂CH₂CH₂CH₂—O—CH₂CH₂CH₂CH₂CH₂CH₃ |
| A-165 | CH₂CH₂CH₂CH₂—O—CH₂CH₂CH₂CH₂CH₂CH₂CH₃ |
| A-166 | CH₂CH₂CH₂CH₂—O—CH₂CH₂CH₂CH₂CH₂CH₂CH₂CH₃ |
| A-167 | CH₂CH₂CH₂CH₂—O—CH(CH₃)₂ |
| A-168 | CH₂CH₂CH₂CH₂—O—C(CH₃)₃ |
| A-169 | CH₂CH₂CH₂CH₂—O—CH₂C(CH₃)₃ |
| A-170 | CH₂CH₂CH₂CH₂—O—CH(CH₃)CH₂C(CH₃)₃ |
| A-171 | CH₂CH₂CH₂CH₂—O—CH(CH₂CH₃)CH₂C(CH₃)₃ |
| A-172 | CH₂CH₂CH₂CH₂—O—CH₂CH(CH₃)CH₂CH(CH₃)₂ |
| A-173 | CH₂CH₂CH₂CH₂—O—CH₂CH(CH₂CH₃)CH₂CH₂CH₃ |
| A-174 | CH₂CH₂CH₂CH₂—O—CH₂CH₂CH(CH₃)CH₂CH(CH₃)₂ |
| A-175 | CH₂CH₂CH₂CH₂—O—CH₂CH₂CH(CH₃)CH₂C(CH₃)₃ |
| A-176 | CH₂CH₂CH₂CH₂—O—CH₂CH₂CH(CH₃)CH₂CH₂CH(CH₃)₂ |
| A-177 | CH₂CH₂CH₂CH₂—O—CH₂CH₂CH(CH₃)CH₂CH₂CH₂CH(CH₃)₂ |
| A-178 | CH₂CH₂CH₂CH₂CH₂—O—CH₃ |
| A-179 | CH₂CH₂CH₂CH₂CH₂—O—CH₂CH₃ |
| A-180 | CH₂CH₂CH₂CH₂CH₂—O—CH₂CH₂CH₃ |
| A-181 | CH₂CH₂CH₂CH₂CH₂—O—CH₂CH₂CH₂CH₃ |
| A-182 | CH₂CH₂CH₂CH₂CH₂—O—CH₂CH₂CH₂CH₂CH₃ |
| A-183 | CH₂CH₂CH₂CH₂CH₂—O—CH₂CH₂CH₂CH₂CH₂CH₃ |
| A-184 | CH₂CH₂CH₂CH₂CH₂—O—CH₂CH₂CH₂CH₂CH₂CH₂CH₃ |
| A-185 | CH₂CH₂CH₂CH₂CH₂—O—CH₂CH₂CH₂CH₂CH₂CH₂CH₂CH₃ |
| A-186 | CH₂CH₂CH₂CH₂CH₂—O—CH(CH₃)₂ |
| A-187 | CH₂CH₂CH₂CH₂CH₂—O—C(CH₃)₃ |
| A-188 | CH₂CH₂CH₂CH₂CH₂—O—CH₂C(CH₃)₃ |
| A-189 | CH₂CH₂CH₂CH₂CH₂—O—CH(CH₃)CH₂C(CH₃)₃ |
| A-190 | CH₂CH₂CH₂CH₂CH₂—O—CH(CH₂CH₃)CH₂C(CH₃)₃ |
| A-191 | CH₂CH₂CH₂CH₂CH₂—O—CH₂CH(CH₃)CH₂CH(CH₃)₂ |
| A-192 | CH₂CH₂CH₂CH₂CH₂—O—CH₂CH(CH₂CH₃)CH₂CH₂CH₃ |
| A-193 | CH₂CH₂CH₂CH₂CH₂—O—CH₂CH₂CH(CH₃)CH₂CH₂CH(CH₃)₂ |
| A-194 | CH₂CH₂CH₂CH₂CH₂—O—CH₂CH₂CH(CH₃)CH₂CH(CH₃)₂ |
| A-195 | CH₂CH₂CH₂CH₂CH₂—O—CH₂CH₂CH(CH₃)CH₂C(CH₃)₃ |
| A-196 | CH₂F |
| A-197 | CH₂Cl |
| A-198 | CH₂Br |
| A-199 | CHF₂ |
| A-200 | CHCl₂ |
| A-201 | CF₃ |
| A-202 | CCl₃ |
| A-203 | CHFCH₃ |
| A-204 | CHClCH₃ |
| A-205 | CH₂CH₂F |
| A-206 | CH₂CH₂Cl |
| A-207 | CH₂CH₂Br |
| A-208 | CCl₂CH₃ |
| A-209 | CF₂CH₃ |
| A-210 | CH₂CHF₂ |
| A-211 | CH₂CHCl₂ |
| A-212 | CH₂CF₃ |
| A-213 | CH₂CCl₃ |
| A-214 | CF₂CF₃ |
| A-215 | CCl₂CCl₃ |
| A-216 | CHFCH₂CH₃ |
| A-217 | CHClCH₂CH₃ |
| A-218 | CH₂CHFCH₃ |
| A-219 | CH₂CHClCH₃ |
| A-220 | CH₂CH₂CH₂F |
| A-221 | CH₂CH₂CH₂Cl |
| A-222 | CH₂CH₂CH₂Br |
| A-223 | CCl₂CH₂CH₃ |
| A-224 | CF₂CH₂CH₃ |
| A-225 | CH₂CH₂CHF₂ |
| A-226 | CH₂CH₂CHCl₂ |
| A-227 | CH₂CH₂CF₃ |
| A-228 | CH₂CH₂CCl₃ |
| A-229 | CF₂CF₂CF₃ |
| A-230 | CCl₂CCl₂CCl₃ |
| A-231 | CH(CH₃)CF₃ |
| A-232 | CH(CH₃)CH₂F |
| A-233 | CH(CH₃)CH₂Cl |
| A-234 | CH(CH₃)CH₂Br |
| A-235 | CH(CH₃)CHF₂ |
| A-236 | CH(CH₃)CHCl₂ |
| A-237 | CH(CH₂F)₂ |
| A-238 | CH(CH₂Cl)₂ |
| A-239 | CH(CH₂Br)₂ |
| A-240 | CH(CHF₂)₂ |
| A-241 | CH(CHCl₂)₂ |
| A-242 | CHFCH₂CH₂CH₃ |
| A-243 | CHClCH₂CH₂CH₃ |
| A-244 | CH₂CHFCH₂CH₃ |
| A-245 | CH₂CHClCH₂CH₃ |
| A-246 | CH₂CH₂CHFCH₃ |
| A-247 | CH₂CH₂CHClCH₃ |
| A-248 | CH₂CH₂CH₂CH₂F |
| A-249 | CH₂CH₂CH₂CH₂Cl |
| A-250 | CH₂CH₂CH₂CH₂Br |
| A-251 | CCl₂CH₂CH₂CH₃ |
| A-252 | CF₂CH₂CH₂CH₃ |
| A-253 | CH₂CH₂CH₂CHF₂ |
| A-254 | CH₂CH₂CH₂CHCl₂ |
| A-255 | CH₂CH₂CH₂CF₃ |
| A-256 | CH₂CH₂CH₂CCl₃ |
| A-257 | CF₂CF₂CF₂CF₃ |
| A-258 | CCl₂CCl₂CCl₂CCl₃ |
| A-259 | CH(CH₃)CH₂CH₂F |
| A-260 | CH(CH₃)CH₂CH₂Cl |
| A-261 | CH(CH₃)CH₂CH₂Br |
| A-262 | CH(CH₃)CH₂CF₃ |
| A-263 | CHFCH₂CH₂CH₂CH₃ |
| A-264 | CHClCH₂CH₂CH₂CH₃ |
| A-265 | CH₂CHFCH₂CH₂CH₃ |
| A-266 | CH₂CHClCH₂CH₂CH₃ |
| A-267 | CH₂CH₂CHFCH₂CH₃ |
| A-268 | CH₂CH₂CHClCH₂CH₃ |
| A-269 | CH₂CH₂CH₂CHFCH₃ |
| A-270 | CH₂CH₂CH₂CHClCH₃ |
| A-271 | CH₂CH₂CH₂CH₂CH₂F |
| A-272 | CH₂CH₂CH₂CH₂CH₂Cl |
| A-273 | CH₂CH₂CH₂CH₂CH₂Br |
| A-274 | CCl₂CH₂CH₂CH₂CH₃ |
| A-275 | CF₂CH₂CH₂CH₂CH₃ |
| A-276 | CH₂CH₂CH₂CH₂CHF₂ |
| A-277 | CH₂CH₂CH₂CH₂CHCl₂ |
| A-278 | CH₂CH₂CH₂CH₂CF₃ |
| A-279 | CH₂CH₂CH₂CH₂CCl₃ |
| A-280 | CF₂CF₂CF₂CF₂CF₃ |
| A-281 | CCl₂CCl₂CCl₂CCl₂CCl₃ |
| A-282 | CH(CH₃)CH₂CH₂CH₂F |
| A-283 | CH(CH₃)CH₂CH₂CH₂Cl |
| A-284 | CH(CH₃)CH₂CH₂CH₂Br |
| A-285 | CH(CH₃)CH₂CH₂CF₃ |
| A-286 | CHFCH₂CH₂CH₂CH₂CH₃ |
| A-287 | CHClCH₂CH₂CH₂CH₂CH₃ |
| A-288 | CH₂CHFCH₂CH₂CH₂CH₃ |
| A-289 | CH₂CHClCH₂CH₂CH₂CH₃ |
| A-290 | CH₂CH₂CHFCH₂CH₂CH₃ |
| A-291 | CH₂CH₂CHClCH₂CH₂CH₃ |
| A-292 | CH₂CH₂CH₂CHFCH₂CH₃ |
| A-293 | CH₂CH₂CH₂CHClCH₂CH₃ |
| A-294 | CH₂CH₂CH₂CH₂CHFCH₃ |
| A-295 | CH₂CH₂CH₂CH₂CHClCH₃ |
| A-296 | CH₂CH₂CH₂CH₂CH₂CH₂F |
| A-297 | CH₂CH₂CH₂CH₂CH₂CH₂Cl |
| A-298 | CH₂CH₂CH₂CH₂CH₂CH₂Br |
| A-299 | CCl₂CH₂CH₂CH₂CH₂CH₃ |
| A-300 | CF₂CH₂CH₂CH₂CH₂CH₃ |
| A-301 | CH₂CH₂CH₂CH₂CH₂CHF₂ |
| A-302 | CH₂CH₂CH₂CH₂CH₂CHCl₂ |
| A-303 | CH₂CH₂CH₂CH₂CH₂CF₃ |
| A-304 | CH₂CH₂CH₂CH₂CH₂CCl₃ |
| A-305 | CF₂CF₂CF₂CF₂CF₂CF₃ |
| A-306 | CCl₂CCl₂CCl₂CCl₂CCl₂CCl₃ |
| A-307 | CH(CH₃)CH₂CH₂CH₂CH₂F |
| A-308 | CH(CH₃)CH₂CH₂CH₂CH₂Cl |
| A-309 | CH(CH₃)CH₂CH₂CH₂CH₂Br |
| A-310 | CH(CH₃)CH₂CH₂CH₂CF₃ |
| A-311 | CHFCH₂CH₂CH₂CH₂CH₂CH₃ |
| A-312 | CHClCH₂CH₂CH₂CH₂CH₂CH₃ |
| A-313 | CH₂CHFCH₂CH₂CH₂CH₂CH₃ |
| A-314 | CH₂CHClCH₂CH₂CH₂CH₂CH₃ |
| A-315 | CH₂CH₂CH₂CHFCH₂CH₂CH₃ |
| A-316 | CH₂CH₂CH₂CHClCH₂CH₂CH₃ |
| A-317 | CH₂CH₂CH₂CH₂CHFCH₂CH₃ |
| A-318 | CH₂CH₂CH₂CH₂CHClCH₂CH₃ |
| A-319 | CH₂CH₂CH₂CH₂CH₂CHFCH₃ |
| A-320 | CH₂CH₂CH₂CH₂CH₂CHClCH₃ |
| A-321 | CH₂CH₂CH₂CH₂CH₂CH₂CH₂F |
| A-322 | CH₂CH₂CH₂CH₂CH₂CH₂CH₂Cl |
| A-323 | CH₂CH₂CH₂CH₂CH₂CH₂CH₂Br |
| A-324 | CCl₂CH₂CH₂CH₂CH₂CH₂CH₃ |
| A-325 | CF₂CH₂CH₂CH₂CH₂CH₂CH₃ |
| A-326 | CH₂CH₂CH₂CH₂CH₂CH₂CHF₂ |
| A-327 | CH₂CH₂CH₂CH₂CH₂CH₂CHCl₂ |
| A-328 | CH₂CH₂CH₂CH₂CH₂CH₂CF₃ |
| A-329 | CH₂CH₂CH₂CH₂CH₂CH₂CCl₃ |
| A-330 | CF₂CF₂CF₂CF₂CF₂CF₂CF₃ |
| A-331 | CCl₂CCl₂CCl₂CCl₂CCl₂CCl₂CCl₃ |
| A-332 | CH(CH₃)CH₂CH₂CH₂CH₂CH₂F |
| A-333 | CH(CH₃)CH₂CH₂CH₂CH₂CH₂Cl |
| A-334 | CH(CH₃)CH₂CH₂CH₂CH₂CH₂Br |
| A-335 | CH(CH₃)CH₂CH₂CH₂CH₂CF₃ |
| A-336 | CHFCH₂CH₂CH₂CH₂CH₂CH₂CH₃ |
| A-337 | CHClCH₂CH₂CH₂CH₂CH₂CH₂CH₃ |
| A-338 | CH₂CHFCH₂CH₂CH₂CH₂CH₂CH₃ |
| A-339 | CH₂CHClCH₂CH₂CH₂CH₂CH₂CH₃ |
| A-340 | CH₂CH₂CHFCH₂CH₂CH₂CH₂CH₃ |
| A-341 | CH₂CH₂CHClCH₂CH₂CH₂CH₂CH₃ |
| A-342 | CH₂CH₂CH₂CH₂CHFCH₂CH₂CH₃ |
| A-343 | CH₂CH₂CH₂CH₂CHClCH₂CH₂CH₃ |
| A-344 | CH₂CH₂CH₂CH₂CH₂CHFCH₂CH₃ |
| A-345 | CH₂CH₂CH₂CH₂CH₂CHClCH₂CH₃ |
| A-346 | CH₂CH₂CH₂CH₂CH₂CH₂CHFCH₃ |
| A-347 | CH₂CH₂CH₂CH₂CH₂CH₂CHClCH₃ |
| A-348 | CH₂CH₂CH₂CH₂CH₂CH₂CH₂CH₂F |
| A-349 | CH₂CH₂CH₂CH₂CH₂CH₂CH₂CH₂Cl |
| A-350 | CH₂CH₂CH₂CH₂CH₂CH₂CH₂CH₂Br |
| A-351 | CCl₂CH₂CH₂CH₂CH₂CH₂CH₂CH₃ |
| A-352 | CF₂CH₂CH₂CH₂CH₂CH₂CH₂CH₃ |
| A-353 | CH₂CH₂CH₂CH₂CH₂CH₂CH₂CHF₂ |
| A-354 | CH₂CH₂CH₂CH₂CH₂CH₂CH₂CHCl₂ |
| A-355 | CH₂CH₂CH₂CH₂CH₂CH₂CH₂CF₃ |
| A-356 | CH₂CH₂CH₂CH₂CH₂CH₂CH₂CCl₃ |
| A-357 | CF₂CF₂CF₂CF₂CF₂CF₂CF₂CF₃ |
| A-358 | CCl₂CCl₂CCl₂CCl₂CCl₂CCl₂CCl₂CCl₃ |
| A-359 | CH(CH₃)CH₂CH₂CH₂CH₂CH₂CH₂F |
| A-360 | CH(CH₃)CH₂CH₂CH₂CH₂CH₂CH₂Cl |
| A-361 | CH(CH₃)CH₂CH₂CH₂CH₂CH₂CH₂Br |
| A-362 | CH(CH₃)CH₂CH₂CH₂CH₂CH₂CF₃ |
| A-363 | CHFCH₂CH₂CH₂CH₂CH₂CH₂CH₂CH₃ |
| A-364 | CHClCH₂CH₂CH₂CH₂CH₂CH₂CH₂CH₃ |
| A-365 | CH₂CHFCH₂CH₂CH₂CH₂CH₂CH₂CH₃ |
| A-366 | CH₂CHClCH₂CH₂CH₂CH₂CH₂CH₂CH₃ |
| A-367 | CH₂CH₂CHFCH₂CH₂CH₂CH₂CH₂CH₃ |
| A-368 | CH₂CH₂CHClCH₂CH₂CH₂CH₂CH₂CH₃ |
| A-369 | CH₂CH₂CH₂CHFCH₂CH₂CH₂CH₂CH₃ |
| A-370 | CH₂CH₂CH₂CHClCH₂CH₂CH₂CH₂CH₃ |
| A-371 | CH₂CH₂CH₂CH₂CH₂CHFCH₂CH₂CH₃ |
| A-372 | CH₂CH₂CH₂CH₂CH₂CHClCH₂CH₂CH₃ |
| A-373 | CH₂CH₂CH₂CH₂CH₂CH₂CHFCH₂CH₃ |
| A-374 | CH₂CH₂CH₂CH₂CH₂CH₂CHClCH₂CH₃ |
| A-375 | CH₂CH₂CH₂CH₂CH₂CH₂CH₂CHFCH₃ |
| A-376 | CH₂CH₂CH₂CH₂CH₂CH₂CH₂CHClCH₃ |
| A-377 | CH₂CH₂CH₂CH₂CH₂CH₂CH₂CH₂CH₂F |
| A-378 | CH₂CH₂CH₂CH₂CH₂CH₂CH₂CH₂CH₂Cl |
| A-379 | CH₂CH₂CH₂CH₂CH₂CH₂CH₂CH₂CH₂Br |
| A-380 | CCl₂CH₂CH₂CH₂CH₂CH₂CH₂CH₂CH₃ |
| A-381 | CF₂CH₂CH₂CH₂CH₂CH₂CH₂CH₂CH₃ |
| A-382 | CH₂CH₂CH₂CH₂CH₂CH₂CH₂CH₂CHF₂ |
| A-383 | CH₂CH₂CH₂CH₂CH₂CH₂CH₂CH₂CHCl₂ |
| A-384 | CH₂CH₂CH₂CH₂CH₂CH₂CH₂CH₂CF₃ |
| A-385 | CH₂CH₂CH₂CH₂CH₂CH₂CH₂CH₂CCl₃ |
| A-386 | CF₂CF₂CF₂CF₂CF₂CF₂CF₂CF₂CF₃ |
| A-387 | CCl₂CCl₂CCl₂CCl₂CCl₂CCl₂CCl₂CCl₂CCl₃ |
| A-388 | CH(CH₃)CH₂CH₂CH₂CH₂CH₂CH₂CH₂F |
| A-389 | CH(CH₃)CH₂CH₂CH₂CH₂CH₂CH₂CH₂Cl |
| A-390 | CH(CH₃)CH₂CH₂CH₂CH₂CH₂CH₂CH₂Br |
| A-391 | CH(CH₃)CH₂CH₂CH₂CH₂CH₂CH₂CF₃ |
| A-392 | CHFCH₂CH₂CH₂CH₂CH₂CH₂CH₂CH₂CH₃ |
| A-393 | CHClCH₂CH₂CH₂CH₂CH₂CH₂CH₂CH₂CH₃ |
| A-394 | CH₂CHFCH₂CH₂CH₂CH₂CH₂CH₂CH₂CH₃ |
| A-395 | CH₂CHClCH₂CH₂CH₂CH₂CH₂CH₂CH₂CH₃ |
| A-396 | CH₂CH₂CHFCH₂CH₂CH₂CH₂CH₂CH₂CH₃ |
| A-397 | CH₂CH₂CHClCH₂CH₂CH₂CH₂CH₂CH₂CH₃ |
| A-398 | CH₂CH₂CH₂CHFCH₂CH₂CH₂CH₂CH₂CH₃ |
| A-399 | CH₂CH₂CH₂CHClCH₂CH₂CH₂CH₂CH₂CH₃ |
| A-400 | CH₂CH₂CH₂CH₂CHFCH₂CH₂CH₂CH₂CH₃ |
| A-401 | CH₂CH₂CH₂CH₂CHClCH₂CH₂CH₂CH₂CH₃ |
| A-402 | CH₂CH₂CH₂CH₂CH₂CH₂CHFCH₂CH₂CH₃ |
| A-403 | CH₂CH₂CH₂CH₂CH₂CH₂CHClCH₂CH₂CH₃ |
| A-404 | CH₂CH₂CH₂CH₂CH₂CH₂CH₂CHFCH₂CH₃ |
| A-405 | CH₂CH₂CH₂CH₂CH₂CH₂CH₂CHClCH₂CH₃ |
| A-406 | CH₂CH₂CH₂CH₂CH₂CH₂CH₂CH₂CHFCH₃ |
| A-407 | CH₂CH₂CH₂CH₂CH₂CH₂CH₂CH₂CHClCH₃ |
| A-408 | CH₂CH₂CH₂CH₂CH₂CH₂CH₂CH₂CH₂CH₂F |
| A-409 | CH₂CH₂CH₂CH₂CH₂CH₂CH₂CH₂CH₂CH₂Br |
| A-410 | CH₂CH₂CH₂CH₂CH₂CH₂CH₂CH₂CH₂CH₂Cl |
| A-411 | CCl₂CH₂CH₂CH₂CH₂CH₂CH₂CH₂CH₂CH₃ |
| A-412 | CF₂CH₂CH₂CH₂CH₂CH₂CH₂CH₂CH₂CH₃ |
| A-413 | CH₂CH₂CH₂CH₂CH₂CH₂CH₂CH₂CH₂CHF₂ |
| A-414 | CH₂CH₂CH₂CH₂CH₂CH₂CH₂CH₂CH₂CHCl₂ |
| A-415 | CH₂CH₂CH₂CH₂CH₂CH₂CH₂CH₂CH₂CF₃ |
| A-416 | CH₂CH₂CH₂CH₂CH₂CH₂CH₂CH₂CH₂CCl₃ |
| A-417 | CF₂CF₂CF₂CF₂CF₂CF₂CF₂CF₂CF₂CF₃ |
| A-418 | CCl₂CCl₂CCl₂CCl₂CCl₂CCl₂CCl₂CCl₂CCl₂CCl₃ |
| A-419 | CH(CH₃)CH₂CH₂CH₂CH₂CH₂CH₂CH₂CH₂F |
| A-420 | CH(CH₃)CH₂CH₂CH₂CH₂CH₂CH₂CH₂CH₂Cl |
| A-421 | CH(CH₃)CH₂CH₂CH₂CH₂CH₂CH₂CH₂CH₂Br |
| A-422 | CH(CH₃)CH₂CH₂CH₂CH₂CH₂CH₂CH₂CF₃ |
| A-423 | CH═CH₂ |
| A-424 | CH₂CH═CH₂ |
| A-425 | CH═CHCH₃ |
| A-426 | C(CH₃)═CH₂ |
| A-427 | CH₂CH₂CH═CH₂ |
| A-428 | CH₂CH═CHCH₃ |
| A-429 | CH═CHCH₂CH₃ |
| A-430 | CH(CH₃)CH═CH₂ |
| A-431 | C(CH₃)═CHCH₃ |
| A-432 | CH═C(CH₃)₂ |
| A-433 | CH₂CH₂CH₂CH═CH₂ |
| A-434 | CH₂CH₂CH═CHCH₃ |
| A-435 | CH₂CH═CHCH₂CH₃ |
| A-436 | CH═CHCH₂CH₂CH₃ |
| A-437 | CH(CH₃)CH₂CH═CH₂ |
| A-438 | CH₂C(CH₃)═CHCH₃ |
| A-439 | CH₂CH═C(CH₃)₂ |
| A-440 | CH₂CH₂CH₂CH₂CH═CH₂ |
| A-441 | CH₂CH₂CH₂CH═CHCH₃ |
| A-442 | CH₂CH₂CH═CHCH₂CH₃ |
| A-443 | CH₂CH═CHCH₂CH₂CH₃ |
| A-444 | CH═CHCH₂CH₂CH₂CH₃ |
| A-445 | CH(CH₃)CH₂CH₂CH═CH₂ |
| A-446 | CH(CH₃)CH₂CH═CHCH₃ |
| A-447 | CH₂C(CH₃)═CHCH₂CH₃ |
| A-448 | CH₂CH₂CH═C(CH₃)₂ |
| A-449 | CH₂CH₂CH₂CH₂CH₂CH═CH₂ |
| A-450 | CH₂CH₂CH₂CH₂CH═CHCH₃ |
| A-451 | CH₂CH₂CH₂CH═CHCH₂CH₃ |
| A-452 | CH₂CH₂CH═CHCH₂CH₂CH₃ |
| A-453 | CH₂CH═CHCH₂CH₂CH₂CH₃ |
| A-454 | CH═CHCH₂CH₂CH₂CH₂CH₃ |
| A-455 | CH(CH₃)CH₂CH₂CH₂CH═CH₂ |
| A-456 | CH(CH₃)CH₂CH₂CH═CHCH₃ |
| A-457 | C(CH₃)═CHCH₂CH₂CH₂CH₃ |
| A-458 | CH₂CH₂CH₂CH═C(CH₃)₂ |
| A-459 | CH₂CH₂CH₂CH₂CH₂CH₂CH═CH₂ |
| A-460 | CH₂CH₂CH₂CH₂CH₂CH═CHCH₃ |
| A-461 | CH₂CH₂CH₂CH₂CH═CHCH₂CH₃ |
| A-462 | CH₂CH₂CH₂CH═CHCH₂CH₂CH₃ |
| A-463 | CH₂CH₂CH═CHCH₂CH₂CH₂CH₃ |
| A-464 | CH₂CH═CHCH₂CH₂CH₂CH₂CH₃ |
| A-465 | CH═CHCH₂CH₂CH₂CH₂CH₂CH₃ |
| A-466 | CH(CH₃)CH₂CH₂CH₂CH₂CH═CH₂ |
| A-467 | CH(CH₃)CH₂CH₂CH₂CH═CHCH₃ |
| A-468 | C(CH₃)═CHCH₂CH₂CH₂CH₂CH₃ |
| A-469 | CH₂CH₂CH₂CH₂CH═C(CH₃)₂ |
| A-470 | CH₂CH₂CH₂CH₂CH₂CH₂CH₂CH═CH₂ |
| A-471 | CH₂CH₂CH₂CH₂CH₂CH₂CH═CHCH₃ |
| A-472 | CH₂CH₂CH₂CH₂CH₂CH═CHCH₂CH₃ |
| A-473 | CH₂CH₂CH₂CH₂CH═CHCH₂CH₂CH₃ |
| A-474 | CH₂CH₂CH₂CH═CHCH₂CH₂CH₂CH₃ |
| A-475 | CH₂CH₂CH═CHCH₂CH₂CH₂CH₂CH₃ |
| A-476 | CH₂CH═CHCH₂CH₂CH₂CH₂CH₂CH₃ |
| A-477 | CH═CHCH₂CH₂CH₂CH₂CH₂CH₂CH₃ |
| A-478 | CH(CH₃)CH₂CH₂CH₂CH₂CH₂CH═CH₂ |
| A-479 | CH(CH₃)CH₂CH₂CH₂CH₂CH═CHCH₃ |
| A-480 | C(CH₃)═CHCH₂CH₂CH₂CH₂CH₂CH₃ |
| A-481 | CH₂CH₂CH₂CH₂CH₂CH═C(CH₃)₂ |
| A-482 | CH₂CH₂CH₂CH₂CH₂CH₂CH₂CH₂CH═CH₂ |
| A-483 | CH₂CH₂CH₂CH₂CH₂CH₂CH₂CH═CHCH₃ |
| A-484 | CH₂CH₂CH₂CH₂CH₂CH₂CH═CHCH₂CH₃ |
| A-485 | CH₂CH₂CH₂CH₂CH₂CH═CHCH₂CH₂CH₃ |
| A-486 | CH₂CH₂CH₂CH₂CH═CHCH₂CH₂CH₂CH₃ |
| A-487 | CH₂CH₂CH₂CH═CHCH₂CH₂CH₂CH₂CH₃ |
| A-488 | CH₂CH₂CH═CHCH₂CH₂CH₂CH₂CH₂CH₃ |
| A-489 | CH₂CH═CHCH₂CH₂CH₂CH₂CH₂CH₂CH₃ |
| A-490 | CH═CHCH₂CH₂CH₂CH₂CH₂CH₂CH₂CH₃ |
| A-491 | CH(CH₃)CH₂CH₂CH₂CH₂CH₂CH₂CH═CH₂ |
| A-492 | CH(CH₃)CH₂CH₂CH₂CH₂CH₂CH═CHCH₃ |
| A-493 | C(CH₃)═CHCH₂CH₂CH₂CH₂CH₂CH₂CH₃ |
| A-494 | CH₂CH₂CH₂CH₂CH₂CH₂CH═C(CH₃)₂ |
| A-495 | C≡CH |
| A-496 | CH₂C≡CH |
| A-497 | C≡CCH₃ |
| A-498 | CH₂CH₂C≡CH |
| A-499 | CH₂C≡CCH₃ |
| A-500 | C≡CCH₂CH₃ |
| A-501 | CH(CH₃)C≡CH |
| A-502 | CH₂CH₂CH₂C≡CH |
| A-503 | CH₂CH₂C≡CCH₃ |
| A-504 | CH₂C≡CCH₂CH₃ |
| A-505 | C≡CCH₂CH₂CH₃ |
| A-506 | CH(CH₃)CH₂C≡CH |
| A-507 | CH₂CH₂CH₂CH₂C≡CH |
| A-508 | CH₂CH₂CH₂C≡CCH₃ |
| A-509 | CH₂CH₂C≡CCH₂CH₃ |
| A-510 | CH₂C≡CCH₂CH₂CH₃ |
| A-511 | C≡CCH₂CH₂CH₂CH₃ |
| A-512 | CH(CH₃)CH₂CH₂C≡CH |
| A-513 | CH(CH₃)CH₂C≡CCH₃ |
| A-514 | CH₂CH₂CH₂CH₂CH₂C≡CH |
| A-515 | CH₂CH₂CH₂CH₂C≡CCH₃ |
| A-516 | CH₂CH₂CH₂C≡CCH₂CH₃ |
| A-517 | CH₂CH₂C≡CCH₂CH₂CH₃ |
| A-518 | CH₂C≡CCH₂CH₂CH₂CH₃ |
| A-519 | C≡CCH₂CH₂CH₂CH₂CH₃ |
| A-520 | CH(CH₃)CH₂CH₂CH₂C≡CH |
| A-521 | CH(CH₃)CH₂CH₂C≡CCH₃ |
| A-522 | CH(CH₃)CH₂C≡CCH₂CH₃ |
| A-523 | CH₂CH₂CH₂CH₂CH₂CH₂C≡CH |
| A-524 | CH₂CH₂CH₂CH₂CH₂C≡CCH₃ |
| A-525 | CH₂CH₂CH₂CH₂C≡CCH₂CH₃ |
| A-526 | CH₂CH₂CH₂C≡CCH₂CH₂CH₃ |
| A-527 | CH₂CH₂C≡CCH₂CH₂CH₂CH₃ |
| A-528 | CH₂C≡CCH₂CH₂CH₂CH₂CH₃ |
| A-529 | C≡CCH₂CH₂CH₂CH₂CH₂CH₃ |
| A-530 | CH(CH₃)CH₂CH₂CH₂CH₂C≡CH |
| A-531 | CH(CH₃)CH₂CH₂CH₂C≡CCH₃ |
| A-532 | CH₂CH₂CH₂CH₂CH₂CH₂CH₂C≡CH |
| A-533 | CH₂CH₂CH₂CH₂CH₂CH₂C≡CCH₃ |
| A-534 | CH₂CH₂CH₂CH₂CH₂C≡CCH₂CH₃ |
| A-535 | CH₂CH₂CH₂CH₂C≡CCH₂CH₂CH₃ |
| A-536 | CH₂CH₂CH₂C≡CCH₂CH₂CH₂CH₃ |
| A-537 | CH₂CH₂C≡CCH₂CH₂CH₂CH₂CH₃ |
| A-538 | CH₂C≡CCH₂CH₂CH₂CH₂CH₂CH₃ |
| A-539 | C≡CCH₂CH₂CH₂CH₂CH₂CH₂CH₃ |
| A-540 | CH(CH₃)CH₂CH₂CH₂CH₂CH₂C≡CH |
| A-541 | CH(CH₃)CH₂CH₂CH₂CH₂C≡CCH₃ |
| A-542 | CH₂CH₂CH₂CH₂CH₂CH₂CH₂CH₂C≡CH |
| A-543 | CH₂CH₂CH₂CH₂CH₂CH₂CH₂C≡CCH₃ |
| A-544 | CH₂CH₂CH₂CH₂CH₂CH₂C≡CCH₂CH₃ |
| A-545 | CH₂CH₂CH₂CH₂CH₂C≡CCH₂CH₂CH₃ |
| A-546 | CH₂CH₂CH₂CH₂C≡CCH₂CH₂CH₂CH₃ |
| A-547 | CH₂CH₂CH₂C≡CCH₂CH₂CH₂CH₂CH₃ |
| A-548 | CH₂CH₂C≡CCH₂CH₂CH₂CH₂CH₂CH₃ |
| A-549 | CH₂C≡CCH₂CH₂CH₂CH₂CH₂CH₂CH₃ |
| A-550 | C≡CCH₂CH₂CH₂CH₂CH₂CH₂CH₂CH₃ |
| A-551 | CH(CH₃)CH₂CH₂CH₂CH₂CH₂CH₂C≡CH |
| A-552 | CH(CH₃)CH₂CH₂CH₂CH₂CH₂C≡CCH₃ |
| A-553 | CH₂CH₂CH₂CH₂CH₂CN |
| A-554 | CH(CH₃)CH₂CH₂CH₂CN |
| A-555 | CH₂CH(CH₃)CH₂CH₂CN |
| A-556 | CH₂CH₂CH(CH₃)CH₂CN |
| A-557 | CH₂CH₂CH(CH₃)CH₂CN |
| A-558 | CH(CH₃)CH(CH₃)CH₂CN |
| A-559 | CH(CH₃)CH(CH₃)CH₂CN |
| A-560 | CH₂C(CH₃)₂CH₂CN |
| A-561 | CH₂CH₂CH₂CH₂CH₂CH₂CN |
| A-562 | CH(CH₃)CH₂CH₂CH₂CH₂CN |
| A-563 | CH₂CH(CH₃)CH₂CH₂CH₂CN |
| A-564 | CH₂CH₂CH(CH₃)CH₂CH₂CN |
| A-565 | CH₂CH₂CH(CH₃)₂CH₂CH₂CN |
| A-566 | CH₂CH₂CH₂CH(CH₃)CH₂CN |
| A-567 | CH(CH₃)CH(CH₃)CH₂CH₂CN |
| A-568 | CH(CH₃)CH₂CH(CH₃)CH₂CN |
| A-569 | CH₂CH₂C(CH₃)₂CH₂CN |
| A-570 | CH(CH₃)CH₂CH(CH₃)CH₂CN |
| A-571 | CH₂CH₂CH₂CH₂CH₂CH₂CH₂CN |
| A-572 | CH(CH₃)CH₂CH₂CH₂CH₂CH₂CN |
| A-573 | CH₂CH(CH₃)CH₂CH₂CH₂CH₂CN |
| A-574 | CH₂CH₂CH(CH₃)CH₂CH₂CH₂CN |
| A-575 | CH₂CH₂CH₂CH(CH₃)CH₂CH₂CN |
| A-576 | CH₂CH₂CH₂CH₂CH(CH₃)CH₂CN |
| A-577 | CH₂CH₂CH₂CH₂CH(CH₃)CH₂CN |
| A-578 | CH(CH₃)CH(CH₃)CH₂CH₂CH₂CN |
| A-579 | CH₂CH(CH₃)CH(CH₃)CH₂CH₂CN |
| A-580 | CH₂CH₂CH₂C(CH₃)₂CH₂CN |
| A-581 | CH(CH₃)CH₂CH(CH₃)CH₂CH₂CN |
| A-582 | CH₂CH(CH₃)CH(CH₃)CH₂CH₂CN |
| A-583 | CH(CH₃)CH₂CH₂CH(CH₃)CH₂CN |
| A-584 | CH₂CH₂CH₂CH₂CH₂CH₂CH₂CH₂CN |
| A-585 | CH(CH₃)CH₂CH₂CH₂CH₂CH₂CH₂CN |
| A-586 | CH₂CH(CH₃)CH₂CH₂CH₂CH₂CH₂CN |
| A-587 | CH₂CH₂CH(CH₃)CH₂CH₂CH₂CH₂CN |
| A-588 | CH₂CH₂CH₂CH(CH₃)CH₂CH₂CH₂CN |
| A-589 | CH₂CH₂CH₂CH₂CH(CH₃)CH₂CH₂CN |
| A-590 | CH₂CH₂CH₂CH₂CH₂CH(CH₃)CH₂CN |
| A-591 | CH₂CH₂CH₂CH₂C(CH₃)₂CH₂CN |
| A-592 | CH(CH₃)CH(CH₃)CH₂CH₂CH₂CH₂CN |
| A-593 | CH₂CH(CH₃)CH(CH₃)CH₂CH₂CH₂CN |
| A-594 | CH₂CH₂CH₂C(CH₃)₂CH₂CH₂CN |
| A-595 | CH(CH₃)CH₂CH(CH₃)CH₂CH₂CH₂CN |
| A-596 | CH₂CH(CH₃)CH(CH₃)CH₂CH₂CH₂CN |
| A-597 | CH(CH₃)CH₂CH₂CH(CH₃)CH₂CH₂CN |
| A-598 | CH(CH₃)CH₂CH₂CH₂CH(CH₃)CH₂CN |
| A-599 | CH₂CH₂CH₂CH₂CH₂CH₂CH₂CH₂CH₂CN |
| A-600 | CH(CH₃)CH₂CH₂CH₂CH₂CH₂CH₂CH₂CN |
| A-601 | CH₂CH(CH₃)CH₂CH₂CH₂CH₂CH₂CH₂CN |
| A-602 | CH₂CH₂CH(CH₃)CH₂CH₂CH₂CH₂CH₂CN |
| A-603 | CH₂CH₂CH₂CH(CH₃)CH₂CH₂CH₂CH₂CN |
| A-604 | CH₂CH₂CH₂CH₂CH(CH₃)CH₂CH₂CH₂CN |
| A-605 | CH₂CH₂CH₂CH₂CH₂CH₂C(CH₃)₂CH₂CN |
| A-606 | CH(CH₃)CH(CH₃)CH₂CH₂CH₂CH₂CH₂CN |
| A-607 | CH₂CH(CH₃)CH(CH₃)CH₂CH₂CH₂CH₂CN |
| A-608 | CH₂CH₂CH₂C(CH₃)₂CH₂CH₂CH₂CN |
| A-609 | CH(CH₃)CH₂CH(CH₃)CH₂CH₂CH₂CH₂CN |
| A-610 | CH₂CH(CH₃)CH(CH₃)CH₂CH₂CH₂CH₂CN |
| A-611 | CH(CH₃)CH₂CH₂CH(CH₃)CH₂CH₂CH₂CN |
| A-612 | CH(CH₃)CH₂CH₂CH₂C(CH₃)₂CH₂CN |
| A-613 | CH₂CH(CH₃)CH₂CH₂CH(CH₃)₂CH₂CN |
| A-614 | CH(CH₃)CH₂CH₂CH₂CH₂CH(CH₃)CH₂CN |
| A-615 | CH₂CH(CH₃)CH₂CH₂CH₂CH(CH₃)CH₂CN |
| A-616 | CH₂CH₂CH₂CH₂CH₂CH₂CH₂CH₂CH₂CH₂CN |
| A-617 | CH(CH₃)CH₂CH₂CH₂CH₂CH₂CH₂CH₂CH₂CN |
| A-618 | CH₂CH(CH₃)CH₂CH₂CH₂CH₂CH₂CH₂CH₂CN |
| A-619 | CH₂CH₂CH(CH₃)CH₂CH₂CH₂CH₂CH₂CH₂CN |
| A-620 | CH₂CH₂CH(CH₃)CH₂CH₂CH₂CH₂CH₂CN |
| A-621 | CH₂CH₂CH₂CH(CH₃)CH₂CH₂CH₂CH₂CN |
| A-622 | CH₂CH₂CH₂CH₂CH₂C(CH₃)₂CH₂CN |
| A-623 | CH(CH₃)CH(CH₃)CH₂CH₂CH₂CH₂CH₂CH₂CN |
| A-624 | CH₂CH(CH₃)CH(CH₃)CH₂CH₂CH₂CH₂CH₂CN |
| A-625 | CH₂CH₂CH₂C(CH₃)₂CH₂CH₂CH₂CH₂CN |
| A-626 | CH(CH₃)CH₂CH(CH₃)CH₂CH₂CH₂CH₂CH₂CN |
| A-627 | CH₂CH(CH₃)CH(CH₃)CH₂CH₂CH₂CH₂CH₂CN |
| A-628 | CH(CH₃)CH₂CH₂CH(CH₃)CH₂CH₂CH₂CH₂CN |
| A-629 | CH(CH₃)CH₂CH₂CH₂CH(CH₃)CH₂CH₂CH₂CN |
| A-630 | CH(CH₃)CH₂CH₂CH₂CH₂CH(CH₃)CH₂CH₂CN |
| A-631 | CH(CH₃)CH₂CH₂CH₂CH₂CH₂CH(CH₃)CH₂CN |
| A-632 | CH(CH₃)CH₂CH₂CH₂CH₂CH₂C(CH₃)CH₂CN |
| A-633 | CH₂CH(CH₃)CH₂CH₂CH₂CH₂CH(CH₃)CH₂CN |
| A-634 | CH(CH₃)CH₂CH₂CH₂CH₂C(CH₃)₂CH₂CN |
| A-635 | CH₂CH(CH₃)CH₂CH₂CH₂C(CH₃)₂CH₂CN |
| A-636 | CH₂CH₂CH₂CH₂CH₂CH₂CH₂CH₂CH₂CH₂CH₂CN |
| A-637 | CH(CH₃)CH₂CH₂CH₂CH₂CH₂CH₂CH₂CH₂CH₂CN |
| A-638 | CH₂CH(CH₃)CH₂CH₂CH₂CH₂CH₂CH₂CH₂CH₂CN |
| A-639 | CH₂CH₂CH(CH₃)CH₂CH₂CH₂CH₂CH₂CH₂CH₂CN |
| A-640 | CH₂CH₂CH₂CH(CH₃)CH₂CH₂CH₂CH₂CH₂CH₂CN |
| A-641 | CH₂CH₂CH₂CH₂CH(CH₃)CH₂CH₂CH₂CH₂CH₂CN |
| A-642 | CH₂CH₂CH₂CH₂CH₂CH₂CH₂C(CH₃)₂CH₂CN |
| A-643 | CH(CH₃)CH(CH₃)CH₂CH₂CH₂CH₂CH₂CH₂CH₂CN |
| A-644 | CH₂CH(CH₃)CH(CH₃)CH₂CH₂CH₂CH₂CH₂CH₂CN |
| A-645 | CH₂CH₂CH₂C(CH₃)₂CH₂CH₂CH₂CH₂CH₂CN |
| A-646 | CH(CH₃)CH₂CH(CH₃)CH₂CH₂CH₂CH₂CH₂CH₂CN |
| A-647 | CH₂CH(CH₃)CH(CH₃)CH₂CH₂CH₂CH₂CH₂CH₂CN |
| A-648 | CH(CH₃)CH₂CH₂CH(CH₃)CH₂CH₂CH₂CH₂CH₂CN |
| A-649 | CH(CH₃)CH₂CH₂CH₂CH(CH₃)CH₂CH₂CH₂CH₂CN |
| A-650 | CH(CH₃)CH₂CH₂CH₂CH₂CH(CH₃)CH₂CH₂CH₂CN |
| A-651 | CH(CH₃)CH₂CH₂CH₂CH₂CH₂CH(CH₃)CH₂CH₂CN |
| A-652 | CH(CH₃)CH₂CH₂CH₂CH₂CH₂CH₂CH(CH₃)CH₂CN |
| A-653 | CH₂CH(CH₃)CH₂CH₂CH₂CH₂CH(CH₃)CH₂CH₂CN |
| A-654 | CH₂CH(CH₃)CH₂CH₂CH₂CH₂CH(CH₃)CH₂CH₂CN |
| A-655 | CH₂CH₂CH(CH₃)CH₂CH₂CH₂CH₂CH(CH₃)CH₂CN |
| A-656 | CH₂CH(CH₃)CH₂CH₂CH₂CH₂C(CH₃)₂CH₂CN |
| A-657 | CH₂CH₂CH₂CH₂CH₂CH₂CH₂CH₂CH₂CH₂CH₂CH₂CN |
| A-658 | CH(CH₃)CH₂CH₂CH₂CH₂CH₂CH₂CH₂CH₂CH₂CH₂CN |
| A-659 | CH₂CH(CH₃)CH₂CH₂CH₂CH₂CH₂CH₂CH₂CH₂CH₂CN |
| A-660 | CH₂CH₂CH(CH₃)CH₂CH₂CH₂CH₂CH₂CH₂CH₂CH₂CN |
| A-661 | CH₂CH₂CH₂CH(CH₃)CH₂CH₂CH₂CH₂CH₂CH₂CH₂CN |
| A-662 | CH₂CH₂CH₂CH₂CH(CH₃)CH₂CH₂CH₂CH₂CH₂CH₂CN |
| A-663 | CH₂CH₂CH₂CH₂CH₂CH(CH₃)CH₂CH₂CH₂CH₂CH₂CN |
| A-664 | CH₂CH₂CH₂CH₂CH₂CH₂CH(CH₃)CH₂CH₂CH₂CH₂CN |
| A-665 | CH₂CH₂CH₂CH₂CH₂CH₂CH₂CH(CH₃)CH₂CH₂CH₂CN |
| A-666 | CH₂CH₂CH₂CH₂CH₂CH₂CH₂CH₂CH(CH₃)CH₂CH₂CN |
| A-667 | CH₂CH₂CH₂CH₂CH₂CH₂CH₂CH₂CH₂CH(CH₃)CH₂CN |
| A-668 | CH(CH₃)CH(CH₃)CH₂CH₂CH₂CH₂CH₂CH₂CH₂CH₂CN |
| A-669 | CH₂CH(CH₃)CH(CH₃)CH₂CH₂CH₂CH₂CH₂CH₂CH₂CN |
| A-670 | CH₂CH₂CH₂C(CH₃)₂CH₂CH₂CH₂CH₂CH₂CH₂CN |
| A-671 | CH₂CH₂CH₂CH₂CH(CH₃)CH₂CH₂CH₂CH₂CH₂CH₂CN |
| A-672 | CH(CH₃)CH₂CH(CH₃)CH₂CH₂CH₂CH₂CH₂CH₂CH₂CN |
| A-673 | CH(CH₃)CH₂CH₂CH(CH₃)CH₂CH₂CH₂CH₂CH₂CH₂CN |
| A-674 | CH(CH₃)CH₂CH₂CH₂CH(CH₃)CH₂CH₂CH₂CH₂CH₂CN |
| A-675 | CH(CH₃)CH₂CH₂CH₂CH₂CH(CH₃)CH₂CH₂CH₂CH₂CN |
| A-676 | CH(CH₃)CH₂CH₂CH₂CH₂CH₂CH(CH₃)CH₂CH₂CH₂CN |
| A-677 | CH(CH₃)CH₂CH₂CH₂CH₂CH₂CH₂CH(CH₃)CH₂CH₂CN |
| A-678 | CH₂CH(CH₃)CH₂CH₂CH₂CH₂CH(CH₃)CH₂CH₂CH₂CN |
| A-679 | CH₂CH₂CH(CH₃)CH₂CH₂CH₂CH₂CH(CH₃)CH₂CH₂CN |
| A-680 | CH₂CH₂CH₂CH(CH₃)CH₂CH₂CH₂CH(CH₃)CH₂CH₂CN |
| A-681 | CH₂CH(CH₃)CH₂CH₂CH₂CH₂CH₂C(CH₃)₂CH₂CN |
| A-682 | CHFCH₂CN |
| A-683 | CHClCH₂CN |
| A-684 | CCl₂CH₂CN |
| A-685 | CF₂CH₂CN |
| A-686 | CHFCH₂CH₂CN |
| A-687 | CHClCH₂CH₂CN |
| A-688 | CCl₂CH₂CH₂CN |
| A-689 | CF₂CH₂CH₂CN |
| A-690 | CHFCH₂CH₂CH₂CN |
| A-691 | CHClCH₂CH₂CH₂CN |
| A-692 | CCl₂CH₂CH₂CH₂CN |
| A-693 | CF₂CH₂CH₂CH₂CN |
| A-694 | CHFCH₂CH₂CH₂CH₂CN |
| A-695 | CHClCH₂CH₂CH₂CH₂CN |
| A-696 | CCl₂CH₂CH₂CH₂CH₂CN |
| A-697 | CF₂CH₂CH₂CH₂CH₂CN |
| A-698 | CHFCH₂CH₂CH₂CH₂CH₂CN |
| A-699 | CHClCH₂CH₂CH₂CH₂CH₂CN |
| A-700 | CCl₂CH₂CH₂CH₂CH₂CH₂CN |
| A-701 | CF₂CH₂CH₂CH₂CH₂CH₂CN |
| A-702 | CHFCH₂CH₂CH₂CH₂CH₂CH₂CN |
| A-703 | CHClCH₂CH₂CH₂CH₂CH₂CH₂CN |
| A-704 | CCl₂CH₂CH₂CH₂CH₂CH₂CH₂CN |
| A-705 | CF₂CH₂CH₂CH₂CH₂CH₂CH₂CN |
| A-706 | CHFCH₂CH₂CH₂CH₂CH₂CH₂CH₂CN |
| A-707 | CHClCH₂CH₂CH₂CH₂CH₂CH₂CH₂CN |
| A-708 | CCl₂CH₂CH₂CH₂CH₂CH₂CH₂CH₂CN |
| A-709 | CF₂CH₂CH₂CH₂CH₂CH₂CH₂CH₂CN |
| A-710 | CHFCH₂CH₂CH₂CH₂CH₂CH₂CH₂CH₂CN |
| A-711 | CHClCH₂CH₂CH₂CH₂CH₂CH₂CH₂CH₂CN |
| A-712 | CCl₂CH₂CH₂CH₂CH₂CH₂CH₂CH₂CH₂CN |
| A-713 | CF₂CH₂CH₂CH₂CH₂CH₂CH₂CH₂CH₂CN |
| A-714 | CHFCH₂CH₂CH₂CH₂CH₂CH₂CH₂CH₂CH₂CN |
| A-715 | CHClCH₂CH₂CH₂CH₂CH₂CH₂CH₂CH₂CH₂CN |
| A-716 | CCl₂CH₂CH₂CH₂CH₂CH₂CH₂CH₂CH₂CH₂CN |
| A-717 | CF₂CH₂CH₂CH₂CH₂CH₂CH₂CH₂CH₂CH₂CN |

The compounds I are suitable as fungicides. They are distinguished by an outstanding effectiveness against a broad spectrum of phytopathogenic fungi from the classes of the Ascomycetes, Deuteromycetes, Oomycetes and Basidiomycetes, especially from the class of the Oomycetes. Some are systemically effective and they can be used in plant protection as foliar fungicides, fungicides for seed dressing and soil fungicides.

They are particularly important in the control of a multitude of fungi on various cultivated plants, such as wheat, rye, barley, oats, rice, corn, grass, bananas, cotton, soyabeans, coffee, sugar cane, vines, fruits, ornamental plants, and vegetables, such as cucumbers, beans, tomatoes, potatoes and cucurbits, and on the seeds of these plants.

They are especially suitable for controlling the following plant diseases:
Alternaria species on vegetables, rapeseed, sugar beet and fruit and rice (for example A. solani or A. alternata on potato and other plants),
Aphanomyces species on sugar beet and vegetables,
Bipolaris and Drechslera species on corn, cereals, rice and lawns (for example D. teres on barley, D. tritci-repentis on wheat),
Blumeria graminis (powdery mildew) on cereals,
Botrytis cinerea (gray mold) on strawberries, vegetables, flowers and grapevines,
Bremia lactucae on lettuce,
Cercospora species on corn, soybeans, rice and sugar beet (for example C. beticula on sugar beet),
Cochliobolus species on corn, cereals, rice (for example Cochliobolus sativus on cereals, Cochliobolus miyabeanus on rice),
Colletotricum species on soybeans, cotton and other plants (for example C. acutatum on various plants),
Exserohilum speciea on corn,
Erysiphe cichoracearum and Sphaerotheca fuliginea on cucurbits,
Fusarium and Verticillium species (for example V. dahliae) on various plants (for example F. graminearum on wheat),
Gaeumanomyces graminis on cereals,
Gibberella species on cereals and rice (for example Gibberella fujikuroi on rice),
Grainstaining complex on rice,
Helminthosporium species (for example H. graminicola) on corn and rice,
Michrodochium nivale on cereals,
Mycosphaerella species on cereals, bananas and peanuts (M. graminicola on wheat, M. fijiesis on bananas),
Phakopsara pachyrhizi and Phakopsara meibomiae on soybeans,
Phomopsis species on soybeans, sunflowers and grapevines (P. viticola on grapevines, P. helianthii on sunflowers),
Phytophthora infestans on potatoes and tomatoes,
Plasmopara viticola on grapevines,
Podosphaera leucotricha on apples,
Pseudocercosporella herpotrichoides on cereals,
Pseudoperonospora species on hops and cucurbits (for example P. cubenis on cucumbers),
Puccinia species on cereals, corn and asparagus (P. triticina and P. striformis on wheat, P. asparagi on asparagus),
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants,
Rhizoctonia-species (for example R. solani) on cotton, rice, potatoes, lawns, corn, rapeseed, potatoes, sugar beet, vegetables and other plants,
Sclerotinia species (for example S. sclerotiorum) on rapeseed, sunflowers and other plants,
Septoria tritici and Stagonospora nodorum on wheat,
Erysiphe (syn. Uncinula necator) on grapevines,
Setospaeria species on corn and lawns,
Sphacelotheca reilinia on corn,
Thievaliopsis species on soybeans and cotton,
Tilletia species on cereals,
Ustilago species on cereals, corn and sugar beet and
Venturia species (scab) on apples and pears (for example V. inaequalis on apples).

They are particularly suitable for controlling harmful fungi from the class of the Oomycetes, such as Peronospora species, Phytophthora species, Plasmopara viticola and Pseudoperonospora species.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g/100 kg of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds of the formula I can be present in various crystal modifications which may differ in their biological activity. They also form part of the subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

### A Water-Soluble Concentrates (SL, LS)

10 parts by weight of the active compounds are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

### E Suspensions (SC, OD, FS)

In an agitated ball mill, 20 parts by weight of the active compounds are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP, SS, WS)

75 parts by weight of the active compounds are ground in a rotor-stator mill with addition of 25 parts by weight of dispersants, wetting agents and silica gel. Dilution with water gives a stable dispersion or solution of the active compound. The active compound content of the formulation is 75% by weight.

### H Gel Formulations

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

### 2. Products to be Applied Undiluted

### I Dustable Powders (DP, DS)

5 parts by weight of the active compounds are ground finely and mixed intimately with 95 parts by weight of finely divided kaolin. This gives a dustable product having an active compound content of 5% by weight.

### J Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

### K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other active compounds, in particular fungicides, it is in many cases possible to broaden the activity spectrum or to prevent the development of resistance. In many cases, synergistic effects are obtained.

The following list of fungicides, in conjunction with which the compounds according to the invention can be used, is intended to illustrate the possible combinations but does not limit them:

### Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate;

### Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide; azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myciobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

### Nitrogenous Heterocyclyl Compounds

pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol,
pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
   others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

### Carbamates and Dithiocarbamates

dithiocarbamates: ferbam, mancozeb, maneb, metiram, metam, propineb, thiram, zineb, ziram;
   carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;

### Other Fungicides

guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

### SYNTHESIS EXAMPLES

The procedures described in the following synthesis examples were used to prepare further compounds I by appropriate modification of the starting compounds. The compounds thus obtained are listed in the following tables, together with physical data.

### Example 1

### Preparation of ethyl 2-propionyldecanoate

32 g of ethylpropionyl acetate were added to 250 ml of an ethanolic NaOH solution (4.4% strength), and the mixture was stirred at 20-25° C. for 15 min. 63.6 g of 1-iodoctane were then added dropwise, and the entire solution was heated under reflux for 12 hours. The solvent was removed by distillation, the residue was then taken up in ethyl acetate, washed with Water and subsequently dried, and the volatile components were removed. Chromatography on silica gel (cyclohexane:ethyl acetate) of the residue gave 27 g of the title compound as a yellow oil.

### Example 2

### Preparation of 6-ethyl-2-mercapto-5-octylpyrimidin-4-ol

19.9 g of the ester from Ex. 1 were added to 38.9 ml of sodium methoxide solution (30% strength) in 70 ml of methanol, 8.2 g of thiourea were then added and the mixture was heated under reflux for 12 hours. The solvent was removed by distillation, and the residue was then dissolved in water and the solution was adjusted to pH 5 using glacial acetic acid. The resulting precipitate was filtered off, washed with water and dried. This gave 17.4 g of the title compound as yellow crystals.

### Example 3

### Preparation of 6-ethyl-2-methylsulfanyl-5-octylpyrimidin-4-ol

6 g of 2-mercaptopyrimidinol from Ex. 2 were dissolved in 3% strength aqueous NaOH, and 3.46 g of iodomethane were added dropwise at 5-10° C. The reaction solution was stirred at 20 to 25° C. for about 18 hours. The reaction mixture was adjusted to pH 5 using glacial acetic acid and extracted with ethyl acetate. After drying, the solvent was removed from the combined organic phases. This gave 5.6 g of the title compound as a yellow oil.

### Example 4

### Preparation of 4-chloro-6-ethyl-2-methylsulfanyl-5-octylpyrimidine

8.5 g of the pyrimidinol from Ex. 3 were initially charged in 60 ml of POCl₃ and heated under reflux for 30 min. The solvent was distilled off, and the residue was then taken up in water and extracted with ethyl acetate. The combined organic phases were washed with water and then with 10% strength NaHCO₃ solution and subsequently dried, and the solvent was removed. Chromatography on silica gel (cyclohexane/ethyl acetate) gave 7.7 g of the title compound as a light-brown oil.

### Example 5

### Preparation of 6-ethyl-2-methylsulfanyl-5-octylpyrimidin-4-ylamine

7.65 g of the pyrimidine from Ex. 4 and 0.68 g of phenol-4-sulfonic acid were together initially charged in ethanol in an autoclave. 30 ml of liquid ammonia were introduced at 20-25° C., and the autoclave was then stirred under 18.5 bar of autogenous pressure at 130° C. for 57 hours. The reaction mixture was filtered off and the solvent was removed from the filtrate. The residue that remained was taken up in ethyl acetate/water, and the organic phase was separated off and dried and the volatile constituents were removed. The residue gave, after chromatography on silica gel (cyclohexane/ethyl acetate), 4.9 g of the title compound as a colorless, wax-like product.

### Example 6

### Preparation of 6-ethyl-2-methylsulfonyl-5-octylpyrimidin-4-ylamine

1.0 g of the aminopyrimidine from Ex. 5 was dissolved in 15 ml of glacial acetic acid, and 0.06 g of sodium tungstate dihydrate was added at 20-25° C. At 20-30° C., 0.97 ml of 30% strength hydrogen peroxide solution was then added dropwise, and the solution was then stirred at 20-25° C. for 12 hours. Water was added, the mixture was filtered and the residue was then washed with water. The solid was taken up in dichloromethane and dehydrated azeotropically. Removal of the solvent gave 0.45 g of the title compound as colorless crystals of m.p. 90-92° C.

### Example 7

### Preparation of 6-ethyl-5-octyl-2-[1,2,4]-triazol-1-ylpyrimidin-4-ylamine [I-1]

32 mg of sodium hydride were initially charged in 5 ml of dimethyl sulfoxide (DMSO), and a solution of 77 mg of 1,2,4-triazole in 5 ml DMSO was added. After 1 hour of stirring at 20-25° C., a solution of 335 mg of the sulfone from Ex. 6 in 5 ml of DMSO was added dropwise, and the entire solution was stirred at 20-25° C. for 12 hours. Water was then added and the resulting precipitate was filtered off. The precipitate gave, after chromatography on silica gel, 152 mg of the title compound as a colorless crystalline material of m.p. 90-91° C.

### Example 8

### Preparation of 6-ethyl-5-octyl-2-pyrazol-1-yl-pyrimidin-4-ylamine [I-5]

18 mg of sodium hydride were initially charged in 2.5 ml of anhydrous tetrahydrofuran (THF), and a solution of 45 mg of pyrazole in 2.5 ml of anhydrous THF was added.

After 2 hours of stirring at 20-25° C., a solution of 200 mg of the sulfone from Ex. 6 in 2.5 ml of anhydrous THF was added dropwise, and the entire solution was stirred at 20-25° C. for 12 hours. Water was then added, and the solution was extracted with methyl tert-butyl ether (MTBE). The combined organic phases were dried and the solvent was removed. Preparative RP chromatography (CH₃CN/water mixture) gave 66 mg of the title compound as a colorless crystalline material of m.p. 62-63° C.

TABLE I
Compounds of the formula I

| | | | | Phys. Data |
|---|---|---|---|---|
| | | | | (m.p. [° C.]; |
| | | | | ¹H-NMR [δ ppm]; |
| No. | R¹ | R² | R³ | MS M⁺ [m/e]) |
| I-1 | (CH₂)₇CH₃ | CH₂CH₃ | 1,2,4-triazol-1-yl | 94 |
| I-2 | (CH₂)₇CH₃ | CH₂CH₃ | SO₂CH₃ | 90-92 |
| I-3 | (CH₂)₇CH₃ | CH₂CH₃ | SCH₃ | 4.75(s); 2.6(q); |
| | | | | 2.5(s); 2.4(t); |
| | | | | 1.45(m); 1.4-1.35 |
| | | | | (m); 1.3(t); 0.9(t) |
| I-4 | (CH₂)₇CH₃ | CH₂CH₃ | C(O)NH₂ | 7.9(s); 5.9(s); |
| | | | | 5.4(s); 2.75(q); |
| | | | | 2.5(t); 1.5(m); |
| | | | | 1.45-1.2(m); 0.9(t) |
| I-5 | (CH₂)₇CH₃ | CH₂CH₃ | pyrazol-1-yl | 62-63 |
| I-6 | (CH₂)₇CH₃ | CH₃ | SCH₃ | 267.441 |
| I-7 | (CH₂)₇CH₃ | CH₃ | NH₂ | 236.363 |
| I-8 | (CH₂)₇CH₃ | CH₃ | SCH₂C(═CH₂)CH₃ | 307.506 |
| I-9 | (CH₂)₇CH₃ | CH₃ | SCH₂CH═CH₂ | 293.479 |
| I-10 | (CH₂)₇CH₃ | CH₃ | SCH₂C₆H₅ | 343.539 |
| I-11 | (CH₂)₇CH₃ | CH₃ | SH | 253.414 |
| I-12 | (CH₂)₇CH₃ | CH₃ | SO₂CH₃ | 299.439 |
| I-13 | (CH₂)₇CH₃ | CH₃ | SO₂CH₂CH═CH₂ | 325.477 |
| I-14 | (CH₂)₇CH₃ | CH₃ | SO₂CH₂C₆H₅ | 375.537 |
| I-15 | (CH₂)₃C₆H₅ | CH₃ | SCH₃ | 273.404 |
| I-16 | (CH₂)₇CH₃ | CH₃ | CH₃ | 235.375 |
| I-17 | (CH₂)₇CH₃ | CH₃ | OCH₃ | 251.374 |
| I-18 | (CH₂)₃C₆H₅ | CH₃ | SCH₂CH═CH₂ | 299.442 |
| I-19 | (CH₂)₇CH₃ | CH₃ | SCH₂CH₃ | 281.468 |
| I-20 | (CH₂)₇CH₃ | CH₃ | SCH₂CH₂CH₃ | 295.495 |
| I-21 | (CH₂)₇CH₃ | CH₃ | SCH₂CH₂CH═CH₂ | 307.506 |
| I-22 | (CH₂)₇CH₃ | CH₃ | S(CH₂)₂OCH₂CH₃ | 325.521 |
| I-23 | (CH₂)₇CH₃ | CH₃ | S(CH₂)₅CH₃ | 337.576 |
| I-24 | (CH₂)₇CH₃ | CH₃ | SCH(CH₃)₂ | 295.495 |
| I-25 | (CH₂)₇CH₃ | CH₃ | S(CH₂)₂OCH₃ | 311.494 |
| I-26 | (CH₂)₇CH₃ | CH₃ | S(CH₂)₂SCH₂CH₃ | 341.588 |
| I-27 | (CH₂)₇CH₃ | CH₃ | SCH₂CH═CHCH₃ | 307.506 |
| I-28 | (CH₂)₇CH₃ | CH₃ | 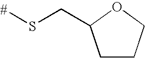 | 337.532 |
| I-29 | (CH₂)₇CH₃ | CH₃ | SCH₂(4-Cl-C₆H₄) | 377.984 |
| I-30 | (CH₂)₇CH₃ | CH₃ | SCH₂(3-Cl-C₆H₄) | 377.984 |
| I-31 | (CH₂)₈CH₃ | CH₃ | SCH₃ | 281.468 |
| I-32 | (CH₂)₃CH₃ | CH₃ | SCH₂CH₃ | 225.36 |
| I-33 | (CH₂)₃CH₃ | CH₃ | SCH₃ | 211.333 |
| I-34 | (CH₂)₄CH₃ | CH₃ | SCH₃ | 225.36 |
| I-35 | (CH₂)₅CH₃ | CH₃ | SCH₃ | 239.387 |
| I-36 | (CH₂)₇CH₃ | CH₃ | SCH₂CH(CH₃)₂ | 309.522 |
| I-37 | (CH₂)₂CH(CH₃)CH₂C(CH₃)₃ | CH₃ | SCH₃ | 281.468 |
| I-38 | (CH₂)₃CH₃ | CH₃ | N(CH₃)₂ | 208.309 |
| I-39 | (CH₂)₇CH₃ | CH₃ | SCH(CH₃)CH₂OCH₃ | 325.521 |
| I-40 | (CH₂)₇CH₃ | CH₃ | N(CH₂CH₃)₂ | 292.471 |
| I-41 | (CH₂)₇CH₃ | CH₃ | C₆H₅ | 297.446 |
| I-42 | (CH₂)₇CH₃ | CH₃ | SCH₂C(O)NH₂ | 310.466 |
| I-43 | (CH₂)₇CH₃ | CH₃ | CH(CH₃)₂ | 263.429 |
| I-44 | CH₂CH(CH₂CH₃)(CH₂)₃CH₃ | CH₃ | SCH₂CH═CH₂ | 293.479 |
| I-45 | CH₂CH(CH₂CH₃)(CH₂)₃CH₃ | CH₃ | SCH₃ | 267.441 |
| I-46 | (CH₂)₇CH₃ | CH₃ | pyrrolidin-1-yl | 290.455 |
| I-47 | (CH₂)₇CH₃ | CH₃ | N(CH₃)₂ | 264.417 |
| I-48 | (CH₂)₇CH₃ | CH₃ | N(CH₃)CH₂CH₃ | 278.444 |
| I-49 | (CH₂)₇CH₃ | CH₃ | CH₂CH₃ | 249.402 |
| I-50 | (CH₂)₇CH₃ | CH₃ | CH₂CH₂CH₃ | 263.429 |
| I-51 | (CH₂)₇CH₃ | CH₃ | OCH(CH₃)₂ | 279.428 |
| I-52 | (CH₂)₂CH(CH₃)CH₂C(CH₃)₃ | CH₃ | N(CH₂CH₃)₂ | 306.498 |
| I-53 | (CH₂)₂CH(CH₃)CH₂C(CH₃)₃ | CH₃ | CH₃ | 249.402 |
| I-54 | (CH₂)₂CH(CH₃)CH₂C(CH₃)₃ | CH₃ | OCH₃ | 265.401 |
| I-55 | (CH₂)₂CH(CH₃)CH₂C(CH₃)₃ | CH₃ | SCH₂CH═CH₂ | 307.506 |
| I-56 | (CH₂)₇CH₃ | CH₃ | morpholin-1-yl | 306.454 |
| I-57 | (CH₂)₇CH₃ | CH₃ | 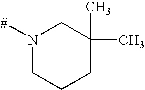 | 332.536 |
| I-58 | (CH₂)₇CH₃ | CH₃ | 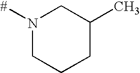 | 318.509 |
| I-59 | (CH₂)₇CH₃ | CH₃ | 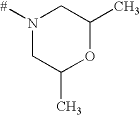 | 334.508 |
| I-60 | (CH₂)₇CH₃ | CH₃ | piperidin-1-yl | 304.482 |
| I-61 | (CH₂)₈CH₃ | CH₃ | N(CH₃)₂ | 278.444 |
| I-62 | (CH₂)₈CH₃ | CH₃ | N(CH₂CH₃)₂ | 306.498 |
| I-63 | (CH₂)₇CH₃ | CH₃ | N(CH₂CH₂CH₃)₂ | 320.525 |
| I-64 | (CH₂)₇CH₃ | CH₃ | N(CH₃)CH₂CH₂CH₃ | 306.498 |
| I-65 | (CH₂)₇CH₃ | CH₃ | N(CH₂CH₂CH₂CH₃)₂ | 348.579 |
| I-66 | (CH₂)₈CH₃ | CH₃ | piperidin-1-yl | 318.509 |
| I-67 | (CH₂)₈CH₃ | CH₃ | pyrrolidin-1-yl | 304.482 |
| I-68 | (CH₂)₈CH₃ | CH₃ | morpholin-1-yl | 320.481 |
| I-69 | (CH₂)₂CH(CH₃)CH₂C(CH₃)₃ | CH₃ | N(CH₃)₂ | 278.444 |
| I-70 | (CH₂)₄CH₃ | CH₃ | N(CH₃)₂ | 222.336 |
| I-71 | (CH₂)₇CH₃ | CH₃ | 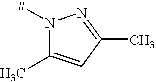 | 315.465 |
| I-72 | (CH₂)₇CH₃ | CH₃ | pyrazol-1-yl | 287.411 |
| I-73 | (CH₂)₇CH₃ | CH₃ | 1,2,4-triazol-1-yl | 288.399 |
| I-74 | (CH₂)₇CH₃ | CH₃ | Cl¹⁾ | 511.586 |
| I-75 | (CH₂)₇CH₃ | CH₃ | OCH₂CH₃ | 265.401 |
| I-76 | (CH₂)₂CH(CH₃)CH₂C(CH₃)₃ | CH₃ | pyrrolidin-1-yl | 304.482 |
| I-77 | (CH₂)₂CH(CH₃)CH₂C(CH₃)₃ | CH₃ | piperidin-1-yl | 318.509 |
| I-78 | CH₂CH(CH₂CH₃)(CH₂)₃CH₃ | CH₃ | N(CH₃)₂ | 264.417 |
| I-79 | CH₂CH(CH₂CH₃)(CH₂)₃CH₃ | CH₃ | pyrrolidin-1-yl | 290.455 |
| I-80 | (CH₂)₉CH₃ | CH₃ | N(CH₃)₂ | 292.471 |
| I-81 | (CH₂)₉CH₃ | CH₃ | N(CH₂CH₃)₂ | 320.525 |
| I-82 | (CH₂)₉CH₃ | CH₃ | pyrrolidin-1-yl | 318.509 |
| I-83 | (CH₂)₉CH₃ | CH₃ | piperidin-1-yl | 332.536 |
| I-84 | —(CH₂)₅— | | NH₂ | 178.239 |
| I-85 | —CH₂CH(CH₃)CH(CH₃)CH₂— | | NH₂ | 192.266 |
| I-86 | —(CH₂)₄— | | NH₂ | 164.212 |
| I-87 | —(CH₂)₃— | | (CH₂)₃CN | 202.261 |
| I-88 | (CH₂)₇CH₃ | CH₃ | CN | 246.358 |
| I-89 | (CH₂)₇CH₃ | CH₃ | C(═NOH)NH₂ | 279.388 |
| I-90 | (CH₂)₇CH₃ | CH₃ | C(═NOCH₃)NH₂ | 293.415 |
| I-91 | (CH₂)₇CH₃ | CH₂CH₃ | CN | 260.385 |

#denotes the bond to the pyrimidine ring
¹⁾mixture with 4-chloro-6-methyl-5-octylpyrimidin-2-ylamine

Examples of the Action Against Harmful Fungi

The fungicidal action of the compounds of the formula I was demonstrated by the following experiments:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent:emulsifier of 99:1. The mixture was then made up to 100 ml with water. This stock solution was diluted with the solvent/emulsifier/water mixture described to the concentration of active compounds stated below.

### Use Example 1

### Activity Against Late Blight of Tomatoes Caused by Phytophthora infestans, Protective Treatment

Leaves of potted tomato plants were sprayed to runoff point with an aqueous suspension having the concentration of active compounds stated below. The next day, the leaves were infected with an aqueous sporangia suspension of Phytophthora infestans. The plants were then placed in a water-vapor-saturated chamber at temperatures between 18 and 20° C. After 6 days, the late blight on the untreated, but infected control plants had developed to such an extent that the infection could be determined visually in %.

In this test, the plants which had been treated with 250 ppm of the compounds I-2, I-4, I-35, I-41 to 46, I-48, I-51 to 55, I-57 to 60, I-62 to 65, I-68, I-71, I-73, I-74, I-79, I-88 or I-91 showed at most 20% infection, whereas the untreated plants were 85-90% infected.

### Use Example 2

### Activity Against the Late Blight Pathogen Phytophthora infestans in the Microtiter Test

50 μl of the required concentration of active compounds were pipetted onto a microtiter plate (MTP). The plate was then inoculated with 50 μl of an aqueous sporangia suspension of Phytophthora infestans. The plates were placed in a water-vapor-saturated chamber at temperatures of 18° C. On the seventh day after the inoculation, the absorption of the MTPs was measured at 405 nm using an absorption photometer. Using the measured parameters, the growth of the control and the blank value, the relative growth in % of the pathogens in the individual active compounds was determined.

In this test, at 125 ppm of the compound I-3 or I-4, the relative growth found was at most 11%.

### Use Example 3

### Protective Activity Against Rice Blast Caused by Pyricularia oryzae in the Microtiter Test

50 μl of the required concentration of active compounds were pipetted onto a microtiter plate (MTP). The plate was then inoculated with 50 μl of an aqueous sporangia suspension of Pyricularia oryzae. The plates were placed in a water-vapor-saturated chamber at temperatures of 18° C. On the seventh day after the inoculation, the absorption of the MTPs was measured at 405 nm using an absorption photometer. Using the measured parameters, the growth of the control and the blank value, the relative growth in % of the pathogens in the individual active compounds was determined. In this test, at 125 ppm of the compound I-3 growth was inhibited completely.

### Use Example 4

### Activity Against peronospora of Grapevines Caused by Plasmopara viticola, 7 Day Protective Treatment

Leaves of potted vines were sprayed to runoff point with an aqueous suspension having the concentration of active compounds stated below. To be able to assess the persistency of the substances, the plants were, after the spray coating had dried on, placed in a greenhouse for 7 days. Only then were the leaves inoculated with an aqueous zoospore suspension of Plasmopara viticola. The vines were then initially placed in a water vapor-saturated chamber at 24° C. for 24 hours and then in a greenhouse at temperatures between 20 and 30° C. for 5 days. After this time, the plants were once more placed in a humid chamber for 16 hours to accelerate the eruption of sporangiospores. The extent of the development of the infection on the undersides of the leaves was then determined visually.

In this test, the plants which had been treated with 500 ppm of the compounds I-1, I-2, I-4, I-10 to 14, I-16, I-17, I-20, I-22, I-23, I-26, I-27, I-28, I-33, I-35 to 53, I-55 to 66, I-68, I-70, I-72 to I-79, bzw. I-80 showed an infection of at most 15%, whereas the untreated plants were 75% infected.

1-14. (canceled)

15. A method for controlling phytopathogenic harmful fungi, wherein the fungi or materials, plants, soil, or seed to be protected against fungal attack are treated with an effective amount of a compound of formula I: 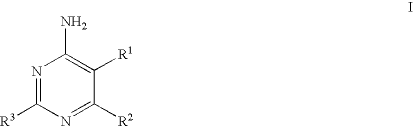
wherein:
R¹ is hydrogen, halogen, cyano, C₁-C₁₄-alkyl, C₁-C₁₄-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, benzyloxy-C₁-C₁₂-alkyl, C₁-C₁₂-alkoxy-C₂-C₁₂-alkenyl, or C₁-C₁₂-alkoxy-C₂-C₁₂-alkynyl;
R² is hydrogen, halogen, cyano, C₁-C₁₂-alkyl, C₁-C₁₂-haloalkyl, C₂-C₁₂-alkenyl, C₂-C₁₂-alkynyl, C₃-C₈-cycloalkyl, C₁-C₁₂-alkoxy, C₁-C₁₂-alkoxy-C₁-C₁₂-alkyl, or C₁-C₁₂-alkylthio-C₁-C₁₂-alkyl,
wherein the carbon chains in R¹ and/or R² may be substituted by one to four identical or different groups R^{α}:
R^{α} is halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, NR^{a}R^{b}, phenyl, or C₁-C₆-alkylphenyl;
R^{a}, R^{b} are independently of one another hydrogen, C₁-C₆-alkyl, C₂-C₆-alkenyl, C₂-C₆-alkynyl, C₃-C₆-cycloalkyl, or C₄-C₆-cycloalkenyl;
wherein the groups R^{α} may be substituted by one to four groups R^{β}:
R^{β} is halogen, cyano, hydroxyl, mercapto, C₁-C₁₀-alkyl, C₁-C₁₀-haloalkyl, C₂-C₁₀-alkenyl, C₂-C₁₀-alkynyl, or C₁-C₆-alkoxy;
R¹ and R² together with the carbon atoms to which they are attached may form a five- to seven-membered ring which may contain one to three identical or different heteroatoms from the group consisting of O, N and S;
R³ is hydrogen, halogen, cyano, hydroxyl, mercapto, azido, C₁-C₈-alkyl, C₂-C₈-alkenyl, C₂-C₈-alkynyl, C₁-C₆-haloalkyl, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, —NR^{c}C(═NR^{c})NR^{a}R^{b}, —NR^{c}C(═O)NR^{a}R^{b}, NR^{a}CN, NR^{a}C(═O)R^{c}, —NR^{a}C(═NOR^{c})R^{c′}, —OC(═O)R^{a}, —C(═NOR^{c})NR^{a}R^{b}, —CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, C(═NOR^{a})NR^{z}R^{b}, C(═NR^{a})NR^{z}R^{b}, —C(═O)NR^{a}NR^{z}R^{b}, —C(═N—NR^{z}R^{c})NR^{a}R^{b}, —C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), —NR^{a}(C(═O)OR^{b}), —NR^{a}(C(═O)—NR^{z}R^{b}), —NR^{a}(C(═NR^{c})R^{b}), NR^{a}(N═CR^{c}R^{b}), NR^{a}—NR^{z}R^{b}, —NR^{z}—OR^{a}, —NR^{a}(C(═NR^{c})—NR^{z}R^{b}), or —NR^{a}(C(═NOR^{c})R^{b}),
D is C₁-C₈-alkyl, C₃-C₈-alkenyl, C₃-C₈-alkynyl, C₁-C₆-haloalkyl, or C₃-C₈-cycloalkyl;
m is 0, 1 or 2;
R^{z} is a group R^{a} which may be attached directly or via a carbonyl group;
R^{c} is one of the groups as defined by R^{a} or R^{b};
a five- or six-membered saturated, partially unsaturated or aromatic mono- or bicyclic heterocycle which contains one to four heteroatoms from the group consisting of O, N and S,
one of the groups G1 or G2 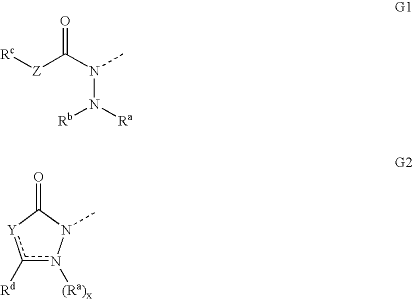
wherein:
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups as defined by R^{a}, R^{b} or, together with the carbon to which it is attached, may be a carbonyl group;
Z is oxygen or N—R^{c};
Y is C(H)—R^{e}, C—R^{e}, N—N(H)—R^{c} or N—R^{c};
R^{e} is halogen, cyano or one of the groups as defined by R^{a} or R^{b}; [symbol]
is a double or a single bond;
wherein the aliphatic, alicyclic or aromatic groups R³, R^{a}, R^{b}, R^{c}, R^{d}, or R^{e} may be partially or fully halogenated or may carry one to four groups R^{A}:
R^{A} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, OH, SH, two vicinal groups R^{A} may be (═O) or (═S), C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N—OA), N(A′)A, N(A′)—C(═O)-A, N(A″)—C(═O)—N(A′)A, S(═O)ₘ-A, S(═O)ₘ—O-A, or S(═O)ₘ—N(A′)A,
A, A′, A″ independently of one another are hydrogen, C₁-C₆-alkyl, C₂-C₆-alkenyl, C₂-C₆-alkynyl, C₃-C₈-cycloalkyl, or C₃-C₈-cycloalkenyl, wherein the groups may be partially or fully halogenated or may be substituted by cyano or C₁-C₄-alkoxy, or A and A′ together with the atoms to which they are attached are a five- or six-membered saturated, partially unsaturated or aromatic heterocycle which contains one to four heteroatoms from the group consisting of O, N and S;
wherein the aliphatic, alicyclic, or aromatic groups R^{A}, A, A′ and A″ may be partially or fully halogenated or may carry one to three groups R^{b}.

16. The method according to claim 15, wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is cyano, mercapto, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, —NR^{c}C(═NR^{c′})NR^{a}R^{b}, —NR^{c}C(═O)NR^{a}R^{b}, —NR^{a}CN, —NR^{a}C(═O)R^{c}, —NR^{a}C(═NOR^{c})R^{c′}, —OC(═O)R^{a}, —C(═NOR^{c})NR^{a}R^{b}, —CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, —C(═O)NR^{a}—NR^{z}R^{b}, —C(═N—NR^{z}R^{c})NR^{a}R^{b}, —C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), —NR^{a}(C(═O)OR^{b}), NR^{a}(C(═O)—NR^{z}R^{b}), —NR^{a}(C(═NR^{c})R^{b}), —NR^{a}(N═CR^{c}R^{b}), —NR^{a}—NR^{z}R^{b}, —NR^{z}—OR^{a}, —NR^{a}(C(═NR^{c})—NR^{z}R^{b}), or —NR^{a}(C(═NOR^{c})R^{b}),
D is C₁-C₈-alkyl, C₃-C₈-alkenyl, C₃-C₈-alkynyl, C₁-C₆-haloalkyl, or C₃-C₈-cycloalkyl;
m is 0, 1, or 2;
R^{z} is the group R^{a} which may be attached directly or via a carbonyl group;
R^{c} is one of the groups as defined by R^{a} or R^{b};
a five- or six-membered saturated, partially unsaturated or aromatic mono- or bicyclic heterocycle which contains one to four heteroatoms from the group consisting of O, N and S,
one of the groups G1 or G2 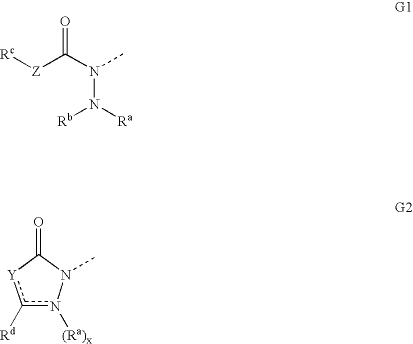
wherein:
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups as defined by R^{a}, R^{b} or, together with the carbon to which it is attached, may be a carbonyl group;
Z is oxygen or N—R^{c};
Y is C(H)—R^{e}, C—R^{e}, N—N(H)—R^{c}, or N—R^{c};
R^{e} is halogen, cyano or one of the groups as defined by R^{a} or R^{b}; [symbol]
is a double or a single bond.

17. The method according to claim 15, wherein R¹ is C₄-C₁₀-alkyl or C₁-C₂-alkoxy-C₁-C₂-alkyl.

18. The method according to claim 15, wherein R² is C₁-C₄-alkyl, or C₁-C₄-alkoxymethyl.

19. The method according to claim 15, wherein:
R³ is a five-membered saturated, partially unsaturated or aromatic monocyclic heterocycle which contains one to four heteroatoms from the group consisting of O, N and S, which may be substituted by R^{A}.

20. The method according to claim 15 wherein:
R³ is cyano, mercapto, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, NR^{c}C(═NR^{c′})NR^{a}R^{b}, —NR^{c}C(═O)NR^{a}R^{b}, —NR^{a}CN, —NR^{a}C(═O)R^{c}, NR^{a}C(═NOR^{c})R^{c′}, OC(═O)R^{a}, —C(═NOR^{c})NR^{a}R^{b}, —CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, —C(═O)NR^{a}—NR^{z}R^{b}, —C(═N—NR^{z}R^{c})NR^{a}R^{b}, —C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), NR^{a}(C(═O)OR^{b}), NR^{a}(C(═O)—NR^{z}R^{b}), NR^{a}(C(═NR^{c})R^{b}), —NR^{a}(N═CR^{c}R^{b}), —NR^{a}—NR^{z}R^{b}, —NR^{z}—OR^{a}, —NR^{a}(C(═NR^{c})—NR^{z}R^{b}), or —NR^{a}(C(═NOR^{c})R^{b});
m is 0 or 2;
D is hydrogen, C₁-C₈-alkyl or C₃-C₈-alkenyl; and
R^{a}, R^{b}, R^{c}, R^{z} are hydrogen or C₁-C₆-alkyl.

21. A process for preparing a compound of formula I: 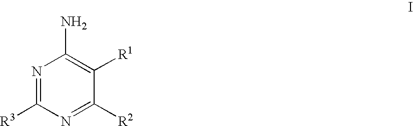
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is cyano or a group attached via a heteroatom,
the process comprising:
a) reacting substituted β-ketoesters of formula II 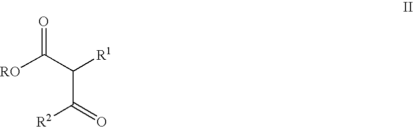
with thiourea of the formula III 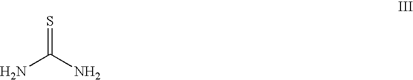
to yield a 2-thio-4-hydroxypyrimidine of formula IV 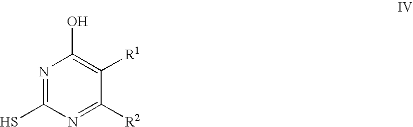
b) reacting said 2-thio-4-hydroxypyrimidine of formula IV with alkylating agents D-X,
wherein D is C₁-C₈-alkyl, C₃-C₈-alkenyl, C₃-C₈-alkynyl, C₁-C₆-haloalkyl, or C₃-C₈-cycloalkyl, to yield a thioether of formula V 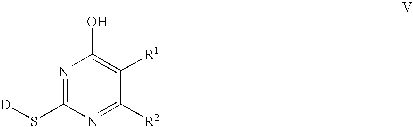
c) halogenating said thioether of formula V to yield a compound of formula VI 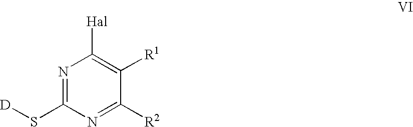
in which Hal is a halogen atom,
d) contacting said compound of formula VI with ammonia to yield a 4-aminopyrimidine of formula I.1, 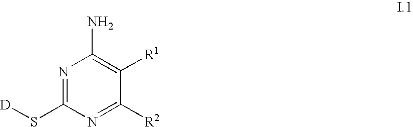
wherein said 4-aminopyrimidine of formula I.1 is optionally oxidized to yield a sulfoxide or a sulfone of formula I.2, and 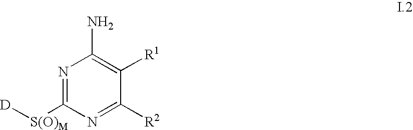
e) reacting a compound of formula I.2 with a compound of formula VII
R³—H VII
or alkali metal, alkaline earth metal or ammonium salts thereof, to yield a compound of formula I, wherein R³ is cyano or a group attached via a heteroatom.

22. A process for preparing a compound of formula I: 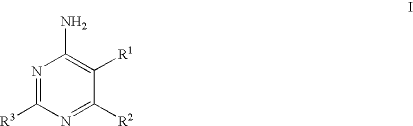
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is C₁-C₈-alkyl, C₂-C₈-alkenyl, C₂-C₈-alkynyl, C₁-C₆-haloalkyl,
the process comprising:
a) reacting substituted β-ketoesters of formula II 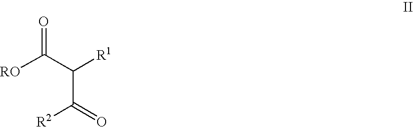
with amidines of formula IIIa 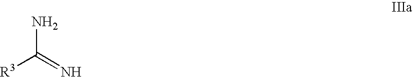
to yield a hydroxypyrimidine of formula Va 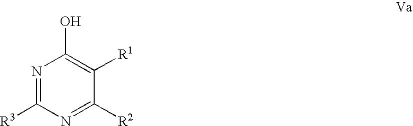
b) halogenating said compound of formula Va to yield a compound of formula VIa; and 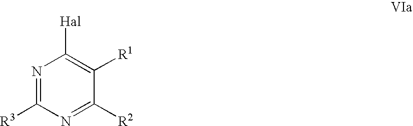
c) reacting said compound of formula VIa with ammonia to yield a compound of formula I.

23. A process for preparing a compound of formula I: 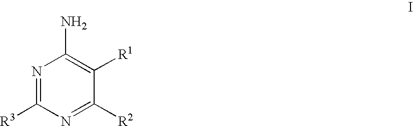
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is a group attached via nitrogen,
the process comprising:
a) reacting substituted β-ketoesters of formula II 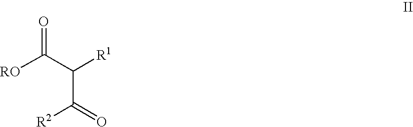
with urea of formula IIIb 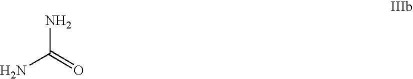
to yield a hydroxypyrimidine of formula Vb 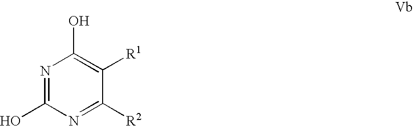
b) halogenating said hydroxypyrimidine of formula Vb to yield a compound of formula VIb 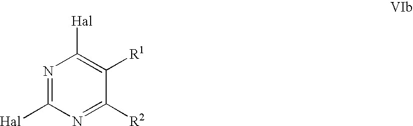
c) reacting said compound of formula VIb with ammonia to yield a diaminopyrimidine; and
d) alkylating or acylating said diaminopyrimidine of step c) to yield a 4-aminopyrimidine of formula I, wherein R³ is a group attached via nitrogen.

24. A process for preparing a compound formula I: 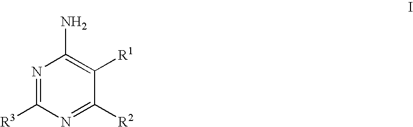
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
the process comprising:
reacting a 2-cyano-4-aminopyrimidine of formula I.3 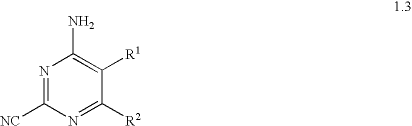
with a compound of formula VII
R³—H VII
wherein R³ is cyano, mercapto, —O-D, —S(O)ₘ-D, —ON═CR^{a}R^{b}, —CR^{c}═NOR^{a}, —NR^{c}N═CR^{a}R^{b}, NR^{a}R^{b}, NR^{c}NR^{a}R^{b}, NOR^{a}, —NR^{c}C(═NR^{c′})NR^{a}R^{b}, —NR^{c}C(═O)NR^{a}R^{b}, —NR^{a}CN, —NR^{a}C(═O)R^{c}, —NR^{a}C(═NOR^{c})R^{c′}, —OC(═O)R^{a}, —C(═NOR^{c})NR^{a}R^{b}, —CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, C(═O)NR^{a}—NR^{z}R^{b}, —C(═N—NR^{z}R^{c})NR^{a}R^{b}, C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), —NR^{a}(C(═O)OR^{b}), NR^{a}(C(═O)—NR^{z}R^{b}), NR^{a}(C(═NR^{c})R^{b}), —NR^{a}(N═CR^{c}R^{b}), —NR^{a}—NR^{z}R^{b}, —NR^{z}—OR^{a}, —NR^{a}(C(═NR^{c})—NR^{z}R^{b}), or —NR^{a}(C(═NOR^{c})R^{b}),
D is C₁-C₈-alkyl, C₃-C₈-alkenyl, C₃-C₈-alkynyl, C₁-C₆-haloalkyl, or C₃-C₈-cycloalkyl;
m is 0, 1, or 2;
R^{z} is the group R^{a} which may be attached directly or via a carbonyl group;
R^{c} is one of the groups as defined by R^{a}, R^{b};
a five- or six-membered saturated, partially unsaturated or aromatic mono- or bicyclic heterocycle which contains one to four heteroatoms from the group consisting of O, N and S,
one of the groups G1 or G2 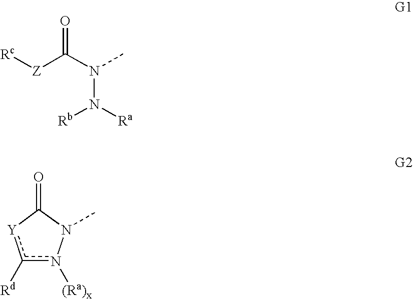
wherein
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups as defined by R^{a}, R^{b} or, together with the carbon to which it is attached, may be a carbonyl group;
Z is oxygen or N—R^{c};
Y is C(H)—R^{e}, C—R^{e}, N—N(H)—R^{c}, or N—R^{c};
R^{e} is halogen, cyano or one of the groups as defined by R^{a} or R^{b}; [symbol]
is a double or a single bond;
or alkali metal, alkaline earth metal or ammonium salts thereof to yield a compound of formula I.

25. A process for preparing a compound of formula I: 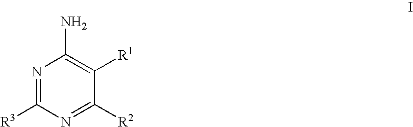
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is cyano or a group R³ attached via a heteroatom,
the process comprising:
a) reacting a compound of formula VIII 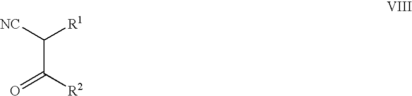
with thiourea to yield a 2-thio-4-hydroxypyrimidine of formula IV 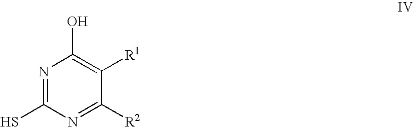
b) reacting said 2-thio-4-hydroxypyrimidine of formula IV with alkylating agents D-X, wherein D is C₁-C₈-alkyl, C₃-C₈-alkenyl, C₃-C₈-alkynyl, C₁-C₆-haloalkyl, or C₃-C₈-cycloalkyl, to yield a thioether of formula V 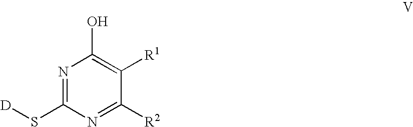
c) halogenating said thioether of formula V to yield a compound of formula VI 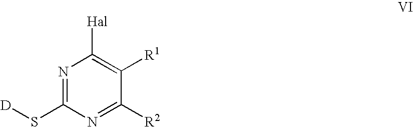
in which Hal is a halogen atom,
d) contacting said compound of formula VI with ammonia to yield a 4-aminopyrimidine of formula I.1, 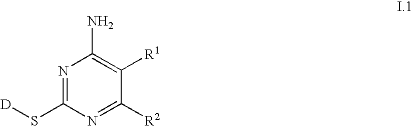
wherein said 4-aminopyrimidine of formula I.1 is optionally oxidized to yield a sulfoxide or a sulfone of formula I.2, and 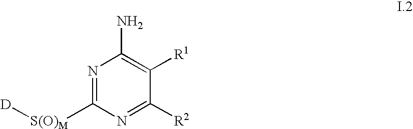
e) reacting a compound of formula I.2 with a compound of formula VII
R³—H VII
or alkali metal, alkaline earth metal or ammonium salts thereof, to yield a compound of formula I.

26. A composition comprising a solid or liquid carrier and a compound of formula I: 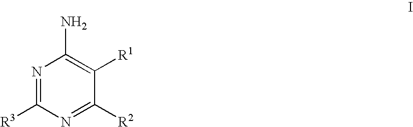
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₈-alkyl;
R² is C₁-C₄-alkyl or C₁-C₈-alkoxy-C₁-C₄-alkyl;
R³ is cyano, mercapto, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, —NR^{c}C(═NR^{c′})NR^{a}R^{b}, —NR^{c}C(═O)NR^{a}R^{b}, —NR^{a}CN, NR^{a}C(═O)R^{c}, —NR^{a}C(═NOR^{c})R^{c′}, —OC(═O)R^{a}, —C(═NOR^{c})NR^{a}R^{b}, —CR^{c}(═NNR^{a}R^{b}), —C(═O)NR^{a}R^{b}, —C(═O)R^{a}, CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, —C(═O)NR^{a}—NR^{z}R^{b}, —C(═N—NR^{z}R^{c})NR^{a}R^{b}, —C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), —NR^{a}(C(═O)OR^{b}), —NR^{a}(C(═O)—NR^{z}R^{b}), —NR^{a}(C(═NR^{c})R^{b}), —NR^{a}(N═CR^{c}R^{b}), —NR^{a}—NR^{z}R^{b}, —NR^{z}—OR^{a}, —NR^{a}(C(═NR^{c})—NR^{z}R^{b}), or —NR^{a}(C(═NOR^{c})R^{b}),
D is C₁-C₈-alkyl, C₃-C₈-alkenyl, C₃-C₈-alkynyl, C₁-C₆-haloalkyl, or C₃-C₈-cycloalkyl;
m is 0, 1, or 2;
R^{z} is the group R^{a} which may be attached directly or via a carbonyl group;
R^{c} is one of the groups as defined by R^{a} or R^{b};
a five- or six-membered saturated, partially unsaturated or aromatic mono- or bicyclic heterocycle which contains one to four heteroatoms from the group consisting of O, N and S,
one of the groups G1 or G2 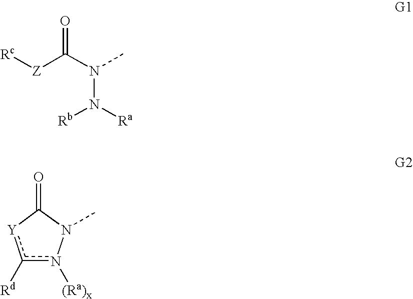
wherein:
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups as defined by R^{a}, R^{b} or, together with the carbon to which it is attached, may be a carbonyl group;
Z is oxygen or N—R^{c};
Y is C(H)—R^{e}, C—R^{e}, N—N(H)—R^{c}, or N—R^{c};
R^{e} is halogen, cyano or one of the groups as defined by R^{a} or R^{b}; [symbol]
is a double or a single bond.

27. Seed comprising the compound of the formula I: 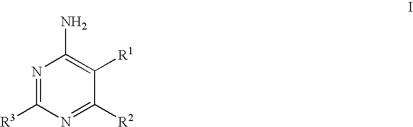
wherein:
R¹ is C₄-C₁₀-alkyl, C₄-C₁₀-haloalkyl, C₄-C₁₀-cyanoalkyl, C₁-C₁₂-alkoxy-C₁-C₁₂-alkyl, or phenyl-C₁-C₉-alkyl;
R² is C₁-C₄-alkyl or C₁-C₈-alkoxy-C₁-C₄-alkyl;
R³ is cyano, mercapto, —O-D, —S(O)ₘ-D, —ON═CR^{a}R^{b}, —CR^{c}═NOR^{a}, —NR^{c}N═CR^{a}R^{b}, —NR^{a}R^{b}, —NR^{c}NR^{a}R^{b}, —NOR^{a}, —NR^{c}C(═NR^{c′})NR^{a}R^{b}, —NR^{c}C(═O)NR^{a}R^{b}, —NR^{a}CN, —NR^{a}C(═O)R^{c}, —NR^{a}C(═NOR^{c})R^{c′}, —OC(═O)R^{a}, —C(═NOR^{c})NR^{a}R^{b}, CR^{c}(═NNR^{a}R^{b}), —C(═O)NR^{a}R^{b}, —C(═O)R^{a}, —CO₂R^{a}, —C(═O)NR^{z}R^{b}, —C(═O)—N—OR^{b}, —C(═S)—NR^{z}R^{b}, —C(═NOR^{a})NR^{z}R^{b}, —C(═NR^{a})NR^{z}R^{b}, —C(═O)NR^{a}—NR^{z}R^{b}, —C(═N—NR^{z}R^{c})NR^{a}R^{b}, C(═NOR^{b})R^{a}, —C(═N—NR^{z}R^{b})R^{a}, —CR^{a}R^{b}—OR^{z}, —CR^{a}R^{b}—NR^{z}R^{c}, —ON(═CR^{a}R^{b}), —NR^{a}(C(═O)R^{b}), NR^{a}(C(═O)OR^{b}), —NR^{a}(C(═O)—NR^{z}R^{b}), —NR^{a}(C(═NR^{c})R^{b}), —NR^{a}(N═CR^{c}R^{b}), —NR^{a}—NR^{z}R^{b}, —NR^{z}—OR^{a}, —NR^{a}(C(═NR^{c})—NR^{z}R^{b}), or —NR^{a}(C(═NOR^{c})R^{b}),
D is C₁-C₈-alkyl, C₃-C₈-alkenyl, C₃-C₈-alkynyl, C₁-C₆-haloalkyl, or C₃-C₈-cycloalkyl;
m is 0, 1, or 2;
R^{z} is the group R^{a} which may be attached directly or via a carbonyl group;
R^{c} is one of the groups as defined by R^{a} or R^{b};
a five- or six-membered saturated, partially unsaturated or aromatic mono- or bicyclic heterocycle which contains one to four heteroatoms from the group consisting of O, N and S,
one of the groups G1 or G2 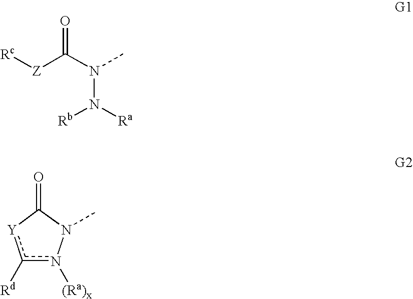
wherein:
x is 0 or 1;
R^{a}, R^{b} are as defined above and, in group G1, together with the nitrogen atom to which they are attached may additionally have the meaning R^{c}-Z-C(R^{d})═N;
R^{d} is halogen, cyano, one of the groups as defined by R^{a}, R^{b} or, together with the carbon to which it is attached, may be a carbonyl group;
Z is oxygen or N—R^{c};
Y is C(H)—R^{e}, C—R^{e}, N—N(H)—R^{c}, or N—R^{c};
R^{e} is halogen, cyano or one of the groups as defined by R^{a} or R^{b}; [symbol]
is a double or a single bond;
in amounts of 1 to 1000 g per 100 kg of seed.
